(19) United States
(12) Patent Application Publication
(10) Pub. No.: US 2022/0267759 A1
(43) Pub. Date: Aug. 25, 2022

(54) METHODS AND COMPOSITIONS FOR SCALABLE POOLED RNA SCREENS WITH SINGLE CELL CHROMATIN ACCESSIBILITY PROFILING

(71) Applicants: New York Genome Center, Inc., New York, NY (US); New York University, New York, NY (US)

(72) Inventors: Neville E. Sanjana, New York, NY (US); Antonino Montalbano, New York, NY (US); Noa Liscovitch-Brauer, Brooklyn, NY (US)

(21) Appl. No.: 17/626,598

(22) PCT Filed: Jul. 12, 2020
(86) PCT No.: PCT/US2020/041738

§ 371 Date: Jan. 12, 2022

Related U.S. Application Data
(60) Provisional application No. 62/873,494, filed on Jul. 12, 2019.

(51) Int. Cl. C12N 15/10; C12N 15/90; C12N 15/11; G01N 1/30; C12Q 1/6806

(52) U.S. Cl. CPC C12N 15/1065; C12N 15/907; C12N 15/11; C12Q 1/6869; C12Q 1/6806; C12N 2310/20; G01N 1/30

(57) ABSTRACT

An in vitro method is provided for analyzing chromatin accessibility and screening RNA of each single cell in a heterologous population (e.g., a library of cells). The method comprises incubating cell nuclei obtained from lysed cells with a transposome complex in a tagmentation buffer, performing reverse transcription wherein each of the RNAs is reverse transcribed to a DNA barcoded with the first barcode; sequencing DNA, which is extracted from digested cell nuclei; and analyzing chromatin accessibility and RNA of the cells. In a further embodiment, the method described comprises performing combinatorial cellular indexing and/or a perturbation step. Additionally, provided are a transposase TnY, buffer(s), and kit(s) for use in the described method.

### GOVERNMENT LICENSE RIGHTS

This invention was made with government support under grant nos. ROOHG008171 and DP2HG010099 awarded by The National Institutes of Health. The government has certain rights in the invention.

### BACKGROUND OF THE INVENTION

Pooled CRISPR screens are widely used to link genes to specific phenotypes, such as drug resistance, cell proliferation, and Mendelian disorders. Recently, CRISPR screens have been combined with single-cell RNA-sequencing technologies connecting multiple genetic perturbations with their effects on gene expression across the transcriptome.

Chromatin accessibility orchestrates trans- and cis-regulatory interactions to control gene expression and is dynamically regulated in cell differentiation and homeostasis. Alterations in chromatin state have been associated with many diseases including several cancers. To assess genome-wide chromatin accessibility, Assay for Transposase-Accessible Chromatin using sequencing (ATAC-seq) was developed and is becoming an essential tool in epigenetics and genome-regulation research. It has been successfully adapted to identify open chromatin and identify regulatory elements across the genome.

Recently, Rubin and collaborators published a method, called Perturb-ATAC, detecting CRISPR guide RNAs and open chromatin sites via a programmable microfluidic device to physically isolate single cells into small chambers (Rubin, A. J. et al. Cell. 2019 Jan. 10; 176(1-2):361-376.e17). This method delivers single cell ATAC-seq data (˜10⁴ fragments per cell), but the throughput per experiment is limited to the 96 chambers of the microfluidic device. Further, Perturb-ATAC targets each gene with a single CRISPR construct, which makes it impossible to measure consistency between perturbations and difficult to know the degree to which off-target effects are responsible for observed phenotypes.

A continuing need in the art exists for scalable and effective methods for investigating chromatin states under RNA-related genetic perturbations (e.g., CRISPR and RNAi), as well as for correlating chromatin accessibility and an RNA profile/transcriptome.

### SUMMARY OF THE INVENTION

In one aspect, an in vitro method is provided for analyzing chromatin accessibility and screening RNA of each single cell in a heterologous population (e.g., a library of cells). The method comprises a tagmentation step, a reverse transcription step, a sequencing step, and an analyzing step.

In the tagmentation step, cell nuclei, each of which comprises DNAs and RNAs from one cell, are obtained from lysed cells and incubated with a transposome complex in a tagmentation buffer. The transposome complex comprises a transposase, a transposon, and a first barcode. During the incubation, the first barcode is ligated to double-stranded DNA at staggered breaks produced by transposase. In certain embodiments, the transposase is TnY or Tn5.

The reverse transcription step allows each of the RNAs (for example, a CRISPR guide RNA, a messenger RNA, a mitochondrial RNA, a microRNA) to be reverse transcribed to a complementary DNA (cDNA). In certain embodiments, the cDNA is barcoded with the first barcode. In certain embodiments, cell nuclei are incubated with reverse transcription primers barcoded with the first barcode or the corresponding antisense sequence thereof, reverse transcriptase, and dNTPs in a reverse transcription buffer. The first barcode may be unique for each cell. In certain embodiments, the reverse transcriptase is REVERTAID™ reverse transcriptase.

During the sequencing step, cell nuclei are digested and DNAs (for example, genomic DNA, genomic DNA fragmented by transposase, and/or cDNA) are extracted and sequenced; while the analyzing step provides chromatin accessibility and RNA sequences of each of the cells.

In a further embodiment, the method provided comprises performing a combinatorial cellular indexing. In certain embodiments, the method comprises transferring the cell nuclei to a first set of compartments prior to the tagmentation step; transferring the cell nuclei to a second set of compartments after the reverse transcription step and prior to the sequencing step; and barcoding each of the DNAs (including tagmented DNAs and cDNAs) with a second barcode. In this method, cell nuclei from the same first-set compartment are transferred to different second-set compartments, whereby sequences acquired and analyzed with the same combination of the first and the second barcodes are identified as being from the same cell. In certain embodiments, the first barcode is unique for each first-set compartment. In certain embodiments, the second barcode is unique for each second-set compartment. A total of n_{c} first-set compartments contain nₙ nuclei per compartment, and a total of m_{c} second-set compartments contain mₙ nuclei per compartment. In certain embodiments, the method further comprises pooling the cell nuclei and randomly distributing the pooled cell nuclei into the second set of compartments, wherein nₙ>>mₙ.

In certain embodiments, the method comprises a perturbation step comprising transducing the cells with one or more vectors and culturing the cells. Each vector comprises a nucleic acid sequence encoding a Cas protein in operative association with a first promoter which controls expression of the Cas protein, and a CRISPR guide RNA coding sequence in operative association with a second promoter which controls transcription thereof. In certain embodiments, the RNA in the reverse transcription step comprises the guide RNAs.

In another aspect, provided is a transposase TnY. Additionally, or alternatively, provided is a cell lysing buffer comprising Tween-20 and Igepal CA630. In certain embodiments, the cell lysing buffer comprises 0.1% Tween-20 and 0.1% Igepal CA630. Also, a fixation buffer is provided comprising about 20% (v/v) ethanol and about 3.1% (v/v) glyoxal at a pH of about 5.0.

In yet another aspect, provided is a kit comprising one or more of the following: a cell lysing buffer, a tagmentation buffer, a transposase, first barcodes, a reverse transcriptase, dNTPs, reverse transcription primers barcoded with the first barcode or the corresponding antisense sequence thereof, a reverse transcription buffer, a cell nuclei digestion buffer, and second barcodes. In certain embodiments, the kit further comprises a vector library. In the library, each vector comprises a nucleic acid sequence encoding a Cas protein in operative association with a first promoter which controls expression of the Cas protein, and a CRISPR guide RNA coding sequence in operative association with a second promoter which controls transcription thereof.

Still other aspects and advantages of these compositions and methods are described further in the following detailed description of the preferred embodiments thereof.

### BRIEF DESCRIPTION OF THE DRAWINGS

FIG. 1A-FIG. 1E show CRISPR screens with single-cell combinatorial indexing assay of transposable and accessible chromatin sequencing (CRISPR-sciATAC) enables the joint capture of chromatin accessibility profiles and CRISPR sgRNAs (FIG. 1A) CRISPR-sciATAC workflow with initial barcoding, nuclei pooling and re-splitting, and then second round barcoding. (FIG. 1B) Comparison of the aggregate chromatin accessibility profiles from K562 cells using Tn5 and TnY transposases and aggregated CRISPR-sciATAC single cell profiles from 11,104 cells. (FIG. 1C) ATAC-seq fragment size distribution from K562 cells of bulk ATAC-seq data, aggregated CRISPR-sciATAC single cell profiles from 11,104 cells and one representative single cell from CRISPR-sciATAC. (FIG. 1D) Number of CRISPR single-guide RNAs (sgRNAs) detected per cell. (FIG. 1E) Proportion of cells bearing 1, 2, or more than 2 sgRNAs.

FIG. 2A-FIG. 2E show a schematic of the CRISPR-sciATAC protocol. (FIG. 2A) CRISPR-sciATAC workflow. BC, barcode. (FIG. 2B) Schematic of ATAC-seq library preparation. (FIG. 2C) Schematic of sgRNA library preparation. (FIG. 2D) CRISPR-sciATAC primer design and library sequencing strategy. (FIG. 2E) sgRNA primer design and library sequencing strategy. Staggered P5 oligos were introduced in the library preparation to introduce sequence diversity. Barcodes 1, 2, and 3 are matched for ATAC-seq and sgRNA libraries, e.g. the ATAC-seq Barcode 1 in well A1 in the 96-well plate where tagmentation is performed has the same DNA sequence as the sgRNA Barcode 1 in well A1 in the 96-well plate where reverse transcription is performed.

FIG. 3A-FIG. 3J show a comparison of TnY and Tn5 transposases. (FIG. 3A) Alignment results of various bacterial transposases with a high-activity variant of Tn5 (Tn5_HA). Amino acids with similar properties are shaded in grey. Multiple alignment was done with ClustalW⁶. (SEQ ID NOs: 14-21, top to bottom) (FIG. 3B) Alignment of V. parahemolyticus transposon end sequences to those of the Tn5 transposon. Tn5 Nextera mosaic end (ME) sequence is also depicted. IE, inside end. OE, outside end. (SEQ ID NOs: 22-26, top to bottom) (FIG. 3C) DNA electrophoresis agarose gel showing migration of ˜700 bp PCR product after incubation with unloaded TnY or loaded with MEDS. (FIG. 3D) Nucleosomal pattern obtained from bulk tagmentation of K562 cells using TnY and a no-transposase negative control. (FIG. 3E) Fragment size distribution and (FIG. 3F) ATAC-seq fragments insertions at transcription start sites (TSS) obtained from bulk tagmentation of K562 cells using TnY. (FIG. 3G-FIG. 3H) Nucleotide frequency plot (upper panel) and DNA sequence logo (lower panel) showing insertion bias of Tn5 (FIG. 3G) and TnY (FIG. H). (FIG. 3I) IGV tracks comparing a TnY bulk ATAC-seq dataset from K562 cells and six previously published K562 Tn5 ATAC-seq datasets [PMID: 30791920, PMID: 28841410, PMID: 26280331] (FIG. 3J) Pearson correlation scores between normalized accessibility averaged over 10 KB genomic bins for the datasets shown in FIG. 3I.

FIG. 4A-FIG. 4C show a species-mixing experiment with minipool CRISPR libraries demonstrates separation of human and mouse single-cell ATAC-seq and sgRNAs. (FIG. 4A) Scatterplot of reads mapping to human or mouse CRISPR libraries (n=1986). (FIG. 4B) Scatterplot of reads mapping to human or mouse genomes (n=721). Outlier cells defined as having more than 10× of the average number of ATAC reads were removed from the visualization (1 cell was removed) (FIG. 4C) The proportion of human ATAC-seq and sgRNA reads mapping to the human and mouse reference genomes and sgRNA libraries (n=496).

FIG. 5A-FIG. 5H show a pooled screen of 21 commonly mutated chromatin modifiers using CRISPR-sciATAC. (FIG. 5A) Chromatin modifiers targeted in the CRISPR library. (FIG. 5B) Mutation load for genes targeted in the chromatin modifier CRISPR library. For each of the chromatin modifiers targeted in the CRISPR library, mutation load is calculated by dividing the number of exonic mutations (in the COSMIC database³) by the gene length. Selected genes represent the top 20 most frequently mutated chromatin modifiers, as defined by mutation load, plus CHD8. (FIG. 5C) sgRNA reads per cell. 15,824 cells had at least 100 sgRNA reads. (FIG. 5D) Representation of sgRNAs within each single cell. The most abundant sgRNA within each cell is colored in blue. (FIG. 5E) Proportion of sgRNAs with the highest read count per cell compared to the number of total sgRNA reads per cell. (FIG. 5F) Unique ATAC-seq reads per cell. 15,364 cells had at least 500 unique reads. (FIG. 5G) Comparison of number of filtered ATAC-seq cells (filtering for 500 unique ATAC-seq reads) with the number sgRNA reads across different sgRNA purity thresholds. (FIG. 5H) Read fraction of different sgRNAs in cells with 500 unique ATAC-seq fragments and 100 sgRNA reads. 11,104 cells with 99% sgRNA reads from a single sgRNA were chosen for further analyses. For the 11,104 cells, overlap of different genomic regions with ATAC-seq peaks called on aggregated single cells²⁷.

FIG. 6A-FIG. 6I show a CRISPR pooled screen enrichment/dropout analysis. (FIG. 6A) Timeline of the depletion and CRISPR-sciATAC screens. (FIG. 6B) Pearson correlation between normalized read counts, all samples in three biological (transduction) replicates. (FIG. 6C) Pearson correlation of the enrichment of library sgRNAs between Week 2 and Early Time Point samples in the three biological replicates. (FIG. 6D) Volcano plot of gene-level enrichment score and Bonferroni-corrected p-values (−log₁₀ q). Genes highlighted in red had |gene-level enrichment|≥0.5 and q≤0.1. (FIG. 6E) Volcano plot of sgRNA-level enrichment (defined as log₂ fold-change between week 2 and the early time point) and significance. sgRNAs highlighted in color have |sgRNA enrichment|≥1 and q≤0.1. Enrichment values are averaged over the three transduction replicates. Colors correspond to the gene function depicted in FIG. 6A. (FIG. 6F) Correlation of gene-level enrichment from this study and from a previous genome-scale CRISPR screen in K562 cells²⁶. The gene-level enrichment is computed as the average enrichment over biological replicates and then over sgRNAs for each gene. (FIG. 6G) Scatter plot of sgRNA enrichment and single cell barcodes obtained in the CRISPR-sciATAC screen. (FIG. 6H) Single cells per sgRNA from the CRISPR-sciATAC experiment in K562 cells. (FIG. 6I) Correlation between cell counts for every pair of sgRNAs targeting the same gene.

FIG. 7A-FIG. 7B show a comparison of CRISPR-sciATAC to Perturb-ATAC and to other sciATAC-seq studies. (FIG. 7A) Number of cells studied in CRISPR-sciATAC and in [PMID: 30580963, PMID: 25953818, PMID: 30166440] (FIG. 7B) Number of ATAC-Seq reads per cell in the original sciATAC-seq paper, sci-CAR (single cell ATAC-seq+RNA expression capture) and CRISPR-sciATAC.

FIG. 8A-FIG. 8C show ATAC-seq fragments counts. The number of ATAC-seq fragments from cells of each sgRNA were compared to the number of fragments in non-targeting cells. There were no significant changes in fragment counts observed (Wilcoxon rank-sum test, significant defined as p≤0.1 following a Bonferroni correction). (FIG. 8A) Scatter plot of ATAC-seq fragments per sgRNA (averaged over cells) and sgRNA enrichment. (FIG. 8B) Scatter plot of peaks called per sgRNA (averaged over cells) and sgRNA enrichment. (FIG. 8C) Scatter plot of the percent of differential peaks per sgRNA and sgRNA enrichment. The fraction of differential peaks is defined as the proportion of peaks that exist only in cells that received that sgRNA and are not found in cells that receive non-targeting sgRNAs. All correlations shown are Pearson correlations.

FIG. 9A-FIG. 9G show CRISPR-sciATAC reveals changes in accessibility at HOX genes following loss of EZH2. (FIG. 9A) Heatmap showing accessibility at histone and DNA modifications for different gene-targeting sgRNA (n=3 sgRNA per gene). (FIG. 9B) Distances in the histone and DNA modifications accessibility profiles shown in a between sgRNAs targeting different genes and sgRNAs targeting the same gene. The distance metric used is 1−(Pearson correlation). (FIG. 9C) Pearson correlation between averaged histone mark Z-score profiles of the indicated number of single cells and the average profile of 400 single cells that received the same perturbation (cells transduced with sgRNAs targeting EZH2 in red, cells transduced with non-targeting sgRNAs in grey). For each cell number, we performed 200 random resamplings (each without replacement) of all 400 cells used for the comparison. (FIG. 9D) UMAP representation of single cells receiving either EZH2 or non-targeting (NT) sgRNAs, calculated based on histone mark differential accessibility profiles in single cells, and the same UMAP representation with single cells colored by TFBS accessibility enrichment scores for CBX2, CBX8, EZH2, POL2B, SIRT6. (FIG. 9E) (top) H3K27me3 ChIP-seq coverage at the HOXA-D loci. (bottom) Changes in accessibility (average number of fragments) at the HOXA-D loci in cells transduced with EZH2-targeting and non-targeting sgRNAs. *** denotes p=0.001. (FIG. 9F) CRISPR-sciATAC fragments mapping to the HOXA locus in cells transduced with EZH2-targeting and non-targeting sgRNAs (n=510 cells per condition). K562 H3K27me3 ChIP-seq coverage is shown at the bottom (blue). The sum of all ATAC fragments over the entire HOXA locus in cells transduced with EZH2-targeting and non-targeting sgRNAs is shown on the right. (FIG. 9G) qPCR results showing expression levels of EZH2, HOXA3, HOXA5, HOXA11A, HOXA13 and HOXD9 for cells transduced with EZH2-targeting sgRNAs.

FIG. 10A-FIG. 10B show differential accessibility in TF binding sites (TFBS). A heatmap was generated showing accessibility at transcription factor binding sites (TFBSs) for the different sgRNAs, including the 50 transcription factors with the most significant differences in accessibility. (FIG. 10A) Distances in the TFBS accessibility profiles from the heatmap between sgRNAs targeting different genes and sgRNAs targeting the same gene. The distance metric used is 1-(Pearson correlation). (FIG. 10B) Scatter plot of guide-level enrichment from the depletion screen and the standard deviation (across sgRNAs) of TFBS accessibility profiles from the heatmap.

FIG. 11A-FIG. 11D show a correlation of down-sampled cell populations with the aggregated pseudo-bulk dataset. Pearson correlation between averaged histone mark Z-score profiles of the indicated number of single cells and the average profile of 400 single cells that received the same perturbation. For each cell number, we performed 200 random resamplings (each without replacement) of all 400 cells used for the comparison. Data is shown for cells transduced with non-targeting sgRNAs (FIG. 11A), EZH2-targeted cells (FIG. 11B), AR/D/A-targeted cells (FIG. 11C) and TET2-targeted cells (FIG. 11D).

FIG. 12A-FIG. 12B show clustering of EZH2 and non-targeting single cells. Hierarchical clustering of EZH2 and non-targeting single cells (one sgRNA for each perturbation) was performed. (FIG. 12A) Confusion matrix showing True Positive Rate (TPR), False Positive Rate (FPR), False Negative Rate (FNR) and True Negative Rate (TNR) for the clustering presented in a when cutting the dendrogram at k=2 (FIG. 12B) The same UMAP representation as shown in FIG. 9D, cells colored by the number of reads per cell.

FIG. 13A-FIG. 13D show ATAC-seq fragments at HOX genes in cells with EZH2 sgRNAs and non-targeting sgRNAs. (FIG. 13A) Gene ontology (GO) terms enriched for genes close to genomic regions with differential accessibility following EZH2 disruption. Shown are selected GO terms with significant enrichment. (FIG. 13B, FIG. 13C, FIG. 13D) CRISPR-sciATAC fragments mapping to the HOXB (FIG. 13B), HOXC (FIG. 13C), and HOXD (FIG. 13B) loci in cells transduced with EZH2-targeting and non-targeting sgRNAs (n=510 cells per condition). K562 H3K27me3 ChIP-seq coverage is shown at the bottom. Summed ATAC fragments over the entire locus in EZH2-targeted and non-targeting aggregated single cells is shown on the right.

FIG. 14A-FIG. 14D show changes in chromatin accessibility at blood cis-eQTLs. (FIG. 14A) Percent of fragments covering at least one blood cis-eQTL in KDM6A-targeted cells. Compared to non-targeting cells, KDM6A-targeted cells have reduced chromatin accessibility at blood cis-eQTLs. (FIG. 14B) Scatter-plot showing relative chromatin accessibility of KDM6A-targeted cells at 7829 blood cis-eQTLs vs. significance (−log₁₀ (chi-square difference in proportion test p-value). Red dots represent eQTLs which are differentially accessible in KDM6A-targeted cells, with nominal significance. (FIG. 14C) Gene ontology (GO) terms enriched for genes whose expression is affected by differentially accessible cis-eQTLs. (FIG. 14D) Four differentially accessible eQTLs highlighted in FIG. 13B. Left, IGV tracks comparing accessibility between KDM6A and non-targeted cells at select eQTLs (arrows). Center, number of fragments in eQTLs for KDM6A or non-targeted cells. Right, local gene expression across different haplotypes at the eQTL, from the GTex (Genotype-Tissue Expression) consortium.

FIG. 15A-FIG. 15F show a CRISPR-sciATAC screen targeting subunits of 16 chromatin remodeling complexes reveals severe disruptions in accessibility upon SWI-SNF disruption. (FIG. 15A) Chromatin remodeling complex subunits/cofactors targeted in the CRISPR library. For each complex, we targeted each gene in the complex with 3 sgRNAs per gene. A heatmap was generated to show accessibility at transcription factor binding sites (TFBSs) for the different chromatin remodeling complexes targeted in the screen. (FIG. 15B) UMAP representation of the genes perturbed in the screen based on the TFBS differential accessibility Z-score profiles. Subunits of the SWI-SNF PBAF complex are labeled with filled circles and gene names. (FIG. 15C) The number of transcription factors with significant differential accessibility (compared to non-targeting controls) following gene targeting. (FIG. 15D) Percent of ATAC fragments in K562 enhancers and in promoters in cells transduced with ARID1A-targeting and non-targeting sgRNAs. Each dot is a single cell. (FIG. 15E) CRISPR-targeted chromatin complex genes with significant differential accessibility at enhancers and/or promoters. (FIG. 15F) Volcano plots showing significant changes in accessibility at TFBSs in cells transduced with ARID1A (left), SMARCA5 (middle) and RCOR1 (right)-targeting sgRNAs. Standardized Z-scores are averaged over single cells. Red dots represent TFBSs with a significant change in accessibility (FDR q≤0.1 and an absolute standardized Z-score>0.25).

FIG. 16A-FIG. 16G Nucleosome dynamics around transcription factor binding sites (TFBSs) following CRISPR targeting of chromatin remodelers. (FIG. 16A) Schematic depicting the computational approach to identify changes in nucleosome positions around TFBSs. (FIG. 16B) (top) Absolute peak shift across 7 TFBS following CRISPR targeting of chromatin remodelers. (bottom) Bubble-plot depicting the peak shifts summarized in the top box-plot for individual TFBS. The color of the bubble corresponds to the peak shift score (nt) and the size of the bubble represents the empirical p-value calculated by a label permutation test. (FIG. 16C) The number of nucleosome expansion and compaction events around TFBSs following CRISPR targeting of chromatin remodelers. (FIG. 16D) Coverage profiles of mono-nucleosomal fragments around AP-1 binding sites in cells transduced with ARID1A-targeting and non-targeting sgRNAs (top) and in cells transduced with EP400-targeting and non-targeting sgRNAs. Dashed lines represent the most highly covered base in each peak. Shaded regions represent s.e.m. (n=3 sgRNAs). (FIG. 16E) Peak shifts in TFBSs located in enhancers and in promoters. Each point is a CRISPR targeted-gene (average of all sgRNAs for that gene). (FIG. 16F) Peak shifts in TFBSs located in enhancers and promoters in SFMBT1-targeted cells (left). Coverage profiles of mono-nucleosome fragments in cells transduced with SFMBT1-targeting and non-targeting sgRNAs around AP-1 binding sites in promoters (top) and in enhancers (bottom). (FIG. 16G) Peak shifts in TFBSs located in enhancers and promoters scores in SMARCB1 targeted cells (left). Coverage profiles of mono-nucleosome fragments in cells transduced with SMARCB1-targeting and non-targeting sgRNAs around RAD21 binding sites in promoters (top) and in enhancers (bottom).

FIG. 17A-FIG. 17C shows nucleosome shifts around TFBSs in enhancers and promoters. (FIG. 17A) Bubble-plot depicting the peak shifts summarized in the top box-plot for individual TFBS in promoters. The color of the bubble corresponds to the peak shift score (nt) and the size of the bubble represents the empirical p-value calculated by a label permutation test. (FIG. 17B) Bubble-plot depicting the peak shifts summarized in the top box-plot for individual TFBS in enhancers. The color of the bubble corresponds to the peak shift score (nt) and the size of the bubble represents the empirical p-value calculated by a label permutation test. (FIG. 17C) Box-plots showing Peak shifts in TFBSs located in enhancers and promoters scores in the different gene knockouts.

FIG. 18 illustrates sequences of oligonucleotides for CRISPR-sciATAC and CRISPR libraries used in the examples (SEQ ID NOs: 27-41, top to bottom).

FIG. 19A and FIG. 19B show tables illustrating gene enrichment from essentiality screen (ETP, early time point) described in the Examples.

FIG. 20 shows the DNA sequence of enzyme TnY (SEQ ID NO: 108).

FIG. 21A and FIG. 21B show a cost comparison between CRISPR-sciATAC and Perturb-ATAC protocols.

FIG. 22 shows a time comparison between CRISPR-sciATAC and Perturb-ATAC protocols.

### DETAILED DESCRIPTION

A scalable in vitro method is provided for analyzing chromatin accessibility and screening RNA (for example, CRISPR guide RNA, microRNA, messenger RNA, non-coding RNAs, mitochondrial RNA, transfer RNA, or ribosomal RNA) of each single cell in a heterologous population (e.g., a library of cells). The method comprises a tagmentation/chromatin accessibility step, a reverse transcription step, a sequencing step and an analyzing step, all described in detail below.

This method permits correlating alterations in chromatin accessibility with RNA screens (for example, transcriptome sequencing, or identification of CRISPR gRNA or microRNA) in a scalable and efficient matter. In certain embodiments, the method may be applied to study diverse phenotypes and diseases influenced by chromatin accessibility and can be combined with large-scale drug screens of small molecule epigenetic modulators to pinpoint mechanisms of drug action. Additionally, provided are compositions and kits that useful in performing the method described herein.

In one embodiment, provided herein is a method that combines pooled CRISPR screens with single cell chromatin accessibility (“CRISPR-sciATAC”). This method simultaneously and reliably captures Assay for Transposase-Accessible Chromatin using sequencing (ATAC-seq) and CRISPR perturbations from single cells. In one embodiment, the method comprises perturbating cells via a CRISPR Cas enzyme and various CRISPR guide RNAs thus generating a heterologous cell population, obtaining cell nuclei from the cells, distributing the cell nuclei into a first set of compartments (for example, a 96-well plate), performing a tagmentation step wherein chromatin DNAs in the cell nuclei are tagmented and ligated with a first barcode which is unique for each first-set compartment, reverse-transcribing CRISPR guide RNAs in the cell nuclei and barcoding the reverse-transcribed cDNAs with the corresponding first barcode, pooling the cell nuclei, redistributing the cell nuclei into a second set of compartments (for example, twelve 96-well plates), optionally digesting the cell nuclei, barcoding the tagmented DNA and the cDNA with a second barcode which is unique for each second-set compartment (for example, during DNA amplification via PCR), sequencing the DNAs, and analyzing results via determining chromatin accessibility of a single cell based on tagmented DNAs barcoded with a combination of the first barcode and the second barcode and via correlating the determined chromatin accessibility status to the guide RNA which perturbates the cell based on the cDNA sequence barcoded with the same combination. In a further embodiment, a total of n_{c} first-set compartments contain nₙ nuclei per compartment, a total of m_{c} second-set compartments contain mₙ nuclei per compartment, and nₙ>>mₙ In one embodiment, a species-mixing experiment shows that CRISPR-sciATAC results in a low doublet rate (for example, about 5% to about 10%). In another embodiment, this method was also applied to identify changes in chromatin accessibility landscapes when perturbing each of the 20 chromatin modifiers most commonly mutated in cancer. These results were integrated with hundreds of existing datasets of transcription factor binding sites and histone modifications. Two specific biological findings were illustrated as examples: (1) Targeting the SWI/SNF subunit ARID1A results in decreased chromatin accessibility at enhancers but not at promoters. Moreover, ARID1A-targeted cells alter nucleosomes positioning at AP-1 transcription factor binding sites demonstrating that CRISPR-sciATAC can deliver high resolution information; and (2) Knockout of the H3K27 methyltransferase EZH2 increases accessibility in heterochromatic regions, including at specific HOX genes.

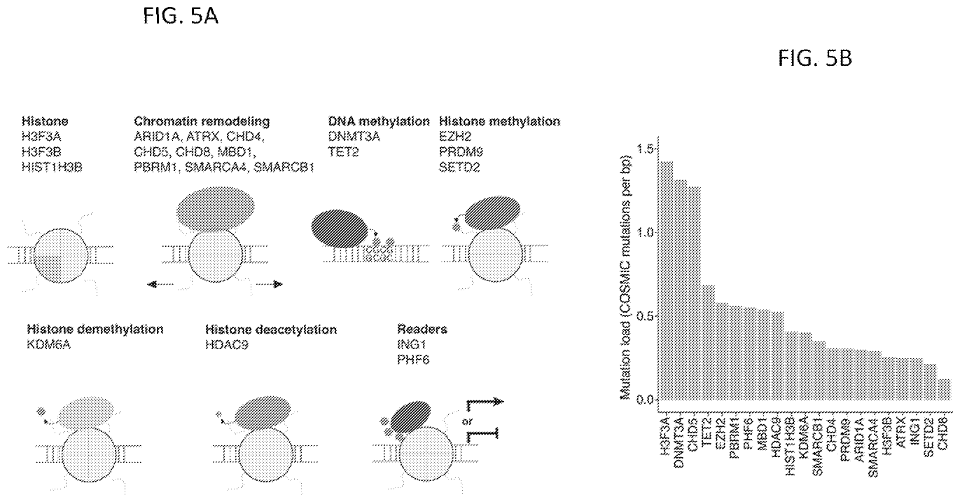

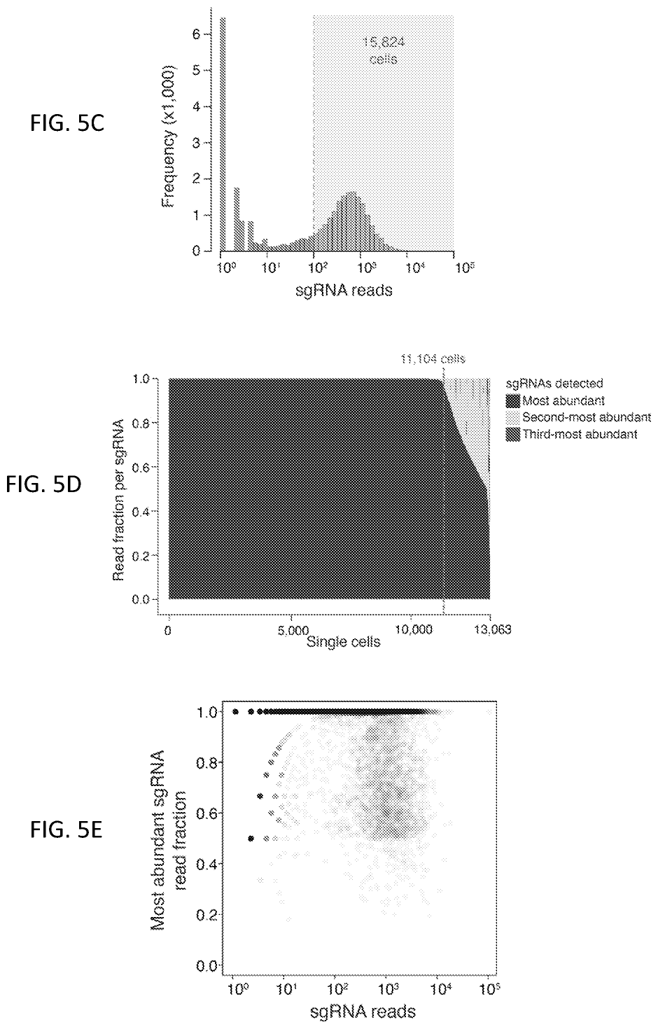

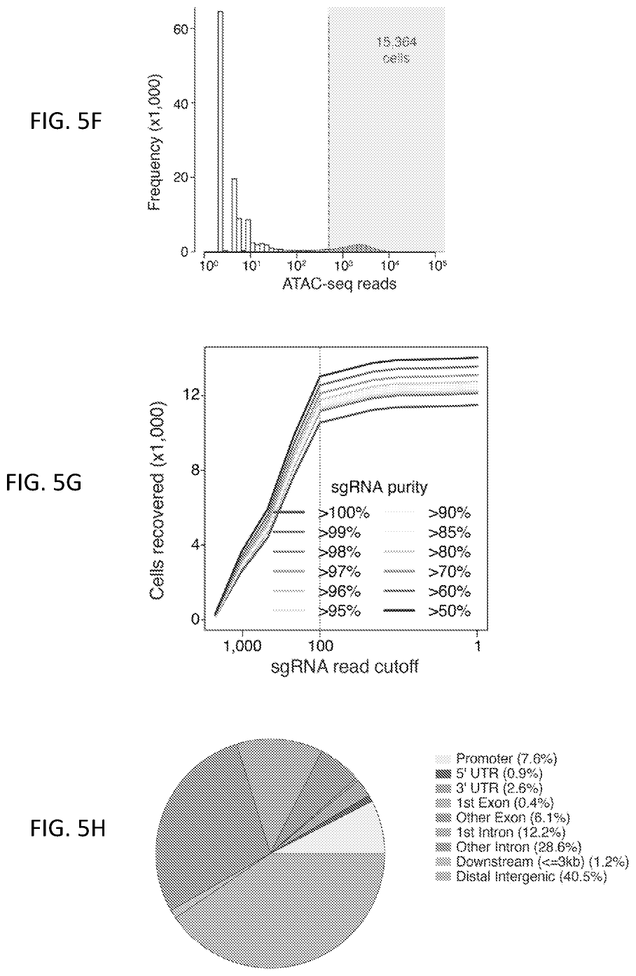

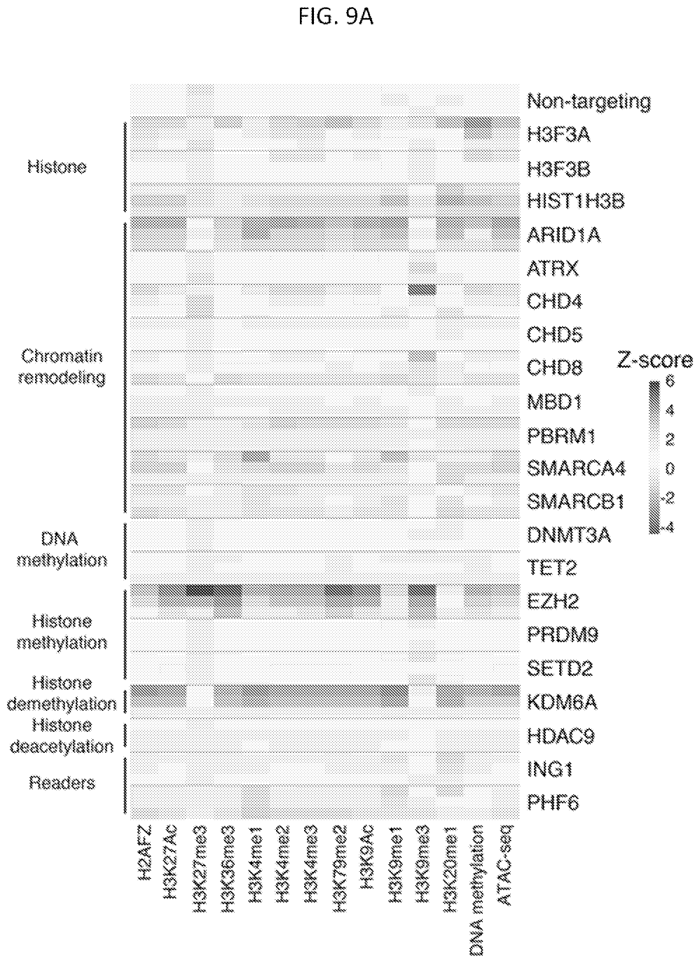

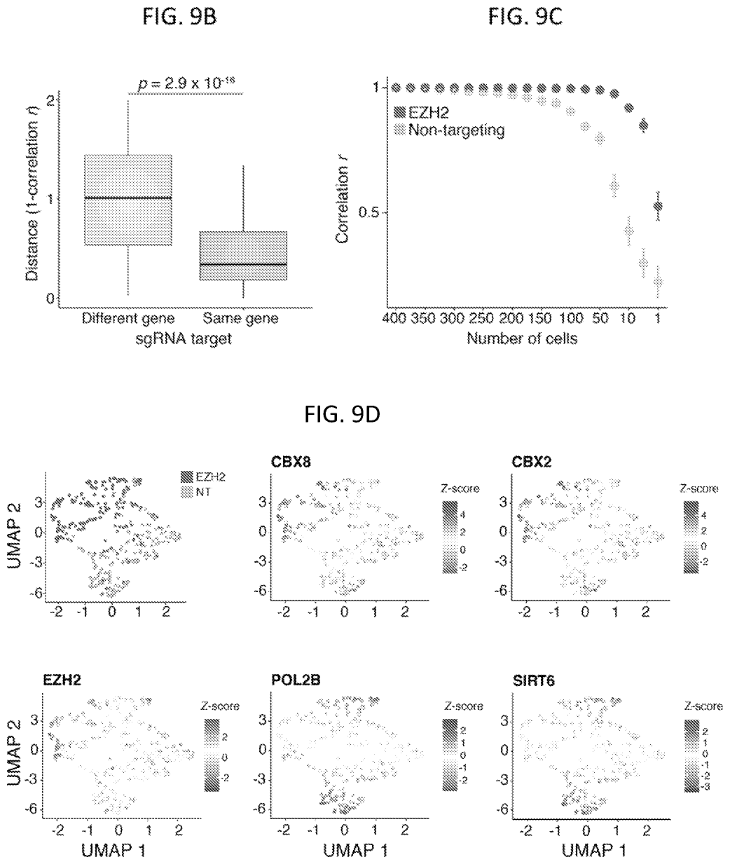

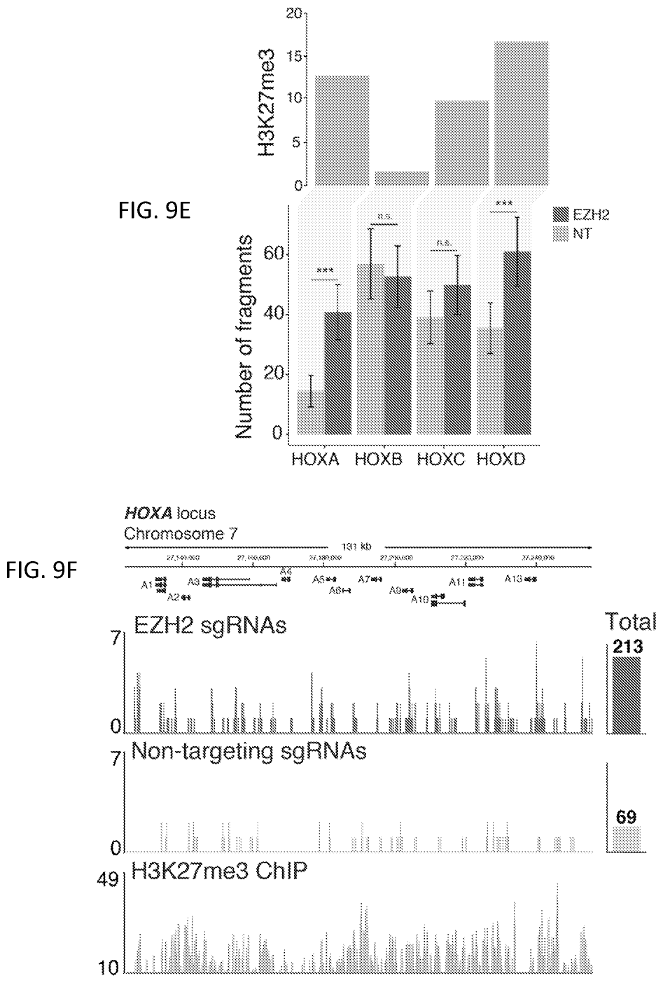

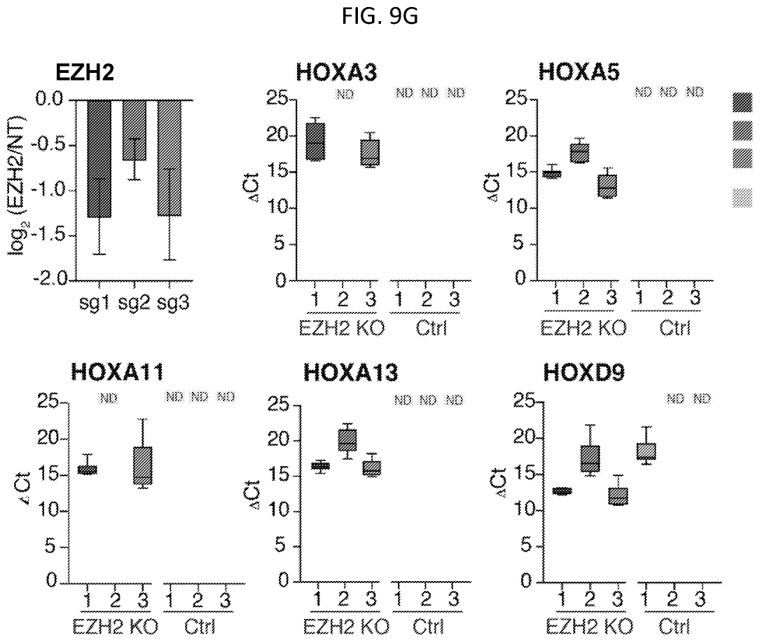

The method described herein (for example, CRISPR-sciATAC) has several important advantages over other known methods, such as Perturb-ATAC (see e.g, Rubin, A. J. et al. Cell. 2019 Jan. 10; 176(1-2):361-376.e17, which is incorporated herein by reference): it can process thousands of cells per plate instead of only 96 cells at a time, which is especially important for large-scale pooled screens; it does not require expensive equipment (e.g. FLUIDIGM device) but instead needs only standard molecular biology equipment; it utilizes multiple perturbations per gene and has high consistency between perturbations (See, for example, FIGS. 5D and 9B). The present method has additional advantages in that it is possible to measure consistency between perturbations and allows one to determine the degree to which off-target effects are responsible for observed phenotypes. In fact, in comparison to prior art methods, the present method can be 20-fold less expensive and 14-fold less time intensive.

This method described herein offers a simple, inexpensive, and highly scalable method to pair pooled RNA screens (for example, pooled CRISPR screens) with single-cell ATAC-seq, and thus expands the screening toolbox with broad applications in cancer biology, differentiation, development, and gene regulation.

### I. Components of the Methods

Components referred to in the methods are described below.

A “nucleic acid” or “nucleic acid sequence”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to nucleic acid sequence encoding proteins, for example that act as transcriptional repressors, antisense molecules, ribozymes, small inhibitory nucleic acid sequences, for example but are not limited to RNA interference (RNAi), short hairpin RNAi (shRNAi), small interfering RNA (siRNA), micro RNAi (mRNAi), antisense oligonucleotides etc.

Ribonucleic acid (RNA) is a polymeric molecule essential in various biological roles in coding, decoding, regulation and expression of genes. As used herein, RNA may refer to a CRISPR guide RNA, a messenger RNA (mRNA), a mitochondrial RNA, a microRNA (miRNA), non-coding RNAs, transfer RNA, ribosomal RNA, short hairpin RNAi (shRNAi), or small interfering RNA (siRNA).

RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. RNAs are the direct products of genes, and these small RNAs can direct enzyme complexes to degrade messenger RNA (mRNA) molecules and thus decrease their activity by preventing translation, via post-transcriptional gene silencing. Moreover, transcription can be inhibited via the pre-transcriptional silencing mechanism of RNA interference, through which an enzyme complex catalyzes DNA methylation at genomic positions complementary to complexed siRNA or miRNA.

As used herein, deoxyribonucleic acid (DNA) is a polymeric molecule formed by deoxyribonucleic acid, including, but not limited to, genomic DNA, double-strand DNA, single-strand DNA, DNA packaged with a histone protein, complementary DNA (cDNA which is reverse-transcribed from a RNA), mitochondrial DNA, and chromosomal DNA.

As used herein, the term “oligo” (i.e., oligonucleotide) refers to short DNA or RNA molecules. In one embodiment, an oligo can be at least about 1 to 500 monomeric components, e.g., nucleotides, in length. In a further embodiment, an oligo can be about 20 to about 80 nucleotides in length. Thus, in various embodiments, an oligo is formed of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99, 100 nucleotides.

The CRISPR-Cas system is a method for functionally inactivating genes in a cell using a CRISPR-associated endonuclease (i.e., Cas, for example, Cas9, Cpf1, or Cas13) to cut the genome or RNA, and a small RNA (guide RNA, gRNA) is used to guide the nuclease to a defined cut site. CRISPR is an abbreviation of clustered regularly interspaced short palindromic repeats.

As used herein, a genome refers to the genetic material of an organism. It consists of DNA (or RNA in RNA viruses). The genome includes both the genes (the coding genomic sequences which code for protein in the organism) and the noncoding DNA (which does not encodes protein in the organism, including but not limited to introns, sequences for non-coding RNAs, regulatory regions such as promoter and enhancer, and repetitive DNA), as well as mitochondrial DNA and chloroplast DNA. Genome editing, or genomic editing, or gene editing, is a type of genetic engineering in which DNA is inserted, deleted, modified or replaced in the genome of an organism. Editing the genome can be achieved using engineered nucleases such as CRISPR-Cas9 (or other CRISPR enzymes), Zinc Finger Nucleases (ZFNs) or Transcription Activator-Like Effector Nucleases (TALENs), RNA interference such as microRNA, transgenesis, viral systems such as rAAV and also transposons. For the most part, gene editing companies can separate genome modifications into one of two experimental categories: loss of function, wherein functional forms of the genome are removed from the system/organism; and gain of function, wherein active (often mutant) forms of the genome are introduced into the system/organism.

The terms “guide RNA,” “gRNA,” “guide,” or “guide sequence,” refer to a nucleic acid sequence which can hybridize to a unique sequence located 3′ or 5′ from a T-rich protospacer-adjacent motif (PAM) in a contiguous region of the genome or a chromosome of a cell, wherein the guide is capable of complexing with Cas protein and providing targeting specificity and binding ability for nuclease activity of Cas. In one embodiment, the guide RNA is about 18 nucleotides (nt) to about 35 nt. In one embodiment, the guide RNA is about 23 nt. The terms “CRISPR RNA spacer,” “spacer,” and “guide RNA coding sequence” are used interchangeably herein and refer to a nucleic acid sequence which encodes a guide RNA. In one embodiment, the spacer is a DNA. In one embodiment, the spacer is about 18 nucleotides (nt) to about 35 nt. In one embodiment, the spacer is about 23 nt. Exemplified spacers and guides can be found in the Examples and Figures.

As used herein, epigenome editing refers to a type of genetic engineering in which the epigenome is modified at specific sites using engineered molecules targeted to those sites (as opposed to whole-genome modifications). Whereas gene editing involves changing the actual DNA sequence itself, epigenetic editing involves modifying and presenting DNA sequences to proteins and other DNA binding factors that influence DNA function.

dNTP stands for deoxyribonucleotide triphosphate. Each dNTP is made up of a phosphate group, a deoxyribose sugar and a nitrogenous base. There are four different dNTPs and can be split into two groups: the purines (including dATP, deoxyadenosine 5′-triphosphate, and dGTP, deoxyguanine 5′-triphosphate) and the pyrimidines (including dTTP, deoxythymidine 5′-triphosphate, and dCTP, deoxycytidine 5′-triphosphate). As used herein, dNTP Mix (also referred to as dNTPs herein) is a mixture (normally in a solution containing sodium salts) of dATP, dCTP, dGTP and dTTP, suitable for use in polymerase chain reaction (PCR), sequencing, fill-in reactions, nick translation, cDNA synthesis, and TdT-tailing reactions. See, for example, www.thermofisher.com/order/catalog/product/18427013.

A “vector” as used herein is a biological or chemical moiety comprising a nucleic acid sequence which can be introduced into an appropriate cell for replication or expression of said the nucleic acid sequence. Common vectors include naked DNA, phage, transposon, plasmids, viral vectors, cosmids (Phillip McClean, www.ndsu.edu/pubweb/˜mcclean/plsc731/cloning/cloning4.htm) and artificial chromosomes (Gong, Shiaoching, et al. “A gene expression atlas of the central nervous system based on bacterial artificial chromosomes.” Nature 425.6961 (2003): 917-925). One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a cell into which they are introduced (e.g., bacterial vectors having a bacterial origin of replication and episomal mammalian vectors). In certain embodiments, the vector is a lentiviral vector. Other vectors (e.g., non-episomal mammalian vectors) are integrated into the genome of a cell upon introduction into the cell, and thereby are replicated along with the cell genome.

A “viral vector” refers to a synthetic or artificial viral particle in which an expression cassette containing a nucleic acid sequence of interest is packaged in a viral capsid or envelope. Examples of viral vector include but are not limited to lentivirus, adenoviruses (Ads), retroviruses (γ-retroviruses and lentiviruses), poxviruses, adeno-associated viruses (AAV), baculoviruses, herpes simplex viruses. In one embodiment, the viral vector is replication defective. A “replication-defective virus” refers to a viral vector, wherein any viral genomic sequences also packaged within the viral capsid or envelope are replication-deficient; i.e., they cannot generate progeny virions but retain the ability to infect cells.

Optionally, the vector further comprises a reporter gene or a nucleic acid encoding a selectable marker, which may include sequences encoding geneticin, hygromicin, ampicillin or purimycin resistance, among others. As used herein, the term “selectable marker” refers to a peptide or polypeptide whose presence can be readily detected in a cell when a selective pressure is applied to the cell. A reporter gene, which is used as an indication of presence of the vector in a cell or not, is readily known by one of skill in the art. For example, the E. coli lacZ gene, the chloramphenicol acetyltransferase (CAT) gene, or a gene encoding a fluorescent protein such as Green fluorescent protein (GFP).

As used herein, “operably linked” sequences or sequences “in operative association” include both expression control sequences that are contiguous with the nucleic acid sequence of interest and expression control sequences that act in trans or at a distance to control the nucleic acid sequence of interest.

In certain embodiments, the vector described herein comprises regulatory sequences. As used herein, the term “regulatory element” or “regulatory sequence” refers to expression control sequences which are contiguous with the nucleic acid sequence of interest and expression control sequences that act in trans or at a distance to control the nucleic acid sequence of interest. As described herein, regulatory elements comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Also, see Goeddel; Gene Expression Technology: Methods in Enzymology 185, Academic Press, San Diego, Calif. (1990). Regulatory sequences include those which direct constitutive expression of a nucleic acid sequence in many types of cells and those which direct expression of the nucleic acid sequence only in certain cells (e.g., tissue-specific regulatory sequences). It will be appreciated by those skilled in the art that the design of the vector can depend on such factors as the choice of the target cell, the level of expression desired, and the like.

By the terms “increase,” “decrease,” “inhibit,” “change,” or a grammatical variation thereof, refer to a variability of at least about 10%, or at least about 20%, or at least about 30%, or at least about 40%, or at least about 50%, or at least about 75%, or at least about 80%, or at least about 90%, from the reference given, unless otherwise specified. By the terms “low” “high” or a grammatical variation thereof, refer to a variability of at least about 10%, or at least about 20%, or at least about 30%, or at least about 40%, or at least about 50%, or at least about 75%, or at least about 80%, or at least about 90%, from the reference given, unless otherwise specified.

The terms “another,” “first, “second,” “third,” “fourth,” “fifth,” and “sixth,” are used throughout this specification as reference terms to distinguish between various forms and components of the compositions and methods, for example, barcodes, compartment sets, or promoters.

The terms “a” or “an” refer to one or more. For example, “a vector” is understood to represent one or more such vectors. As such, the terms “a” (or “an”), “one or more,” and “at least one” are used interchangeably herein.

As used herein, the term “about” or “˜” means a variability of plus or minus 10% from the reference given, unless otherwise specified.

The words “comprise”, “comprises”, and “comprising” are to be interpreted inclusively rather than exclusively, i.e., to include other unspecified components or process steps.

The words “consist”, “consisting”, and its variants, are to be interpreted exclusively, rather than inclusively, i.e., to exclude components or steps not specifically recited.

As used herein, the phrase “consisting essentially of” limits the scope of a described composition or method to the specified materials or steps and those that do not materially affect the basic and novel characteristics of the described or claimed method or composition.

Wherever in this specification, a method or composition is described as “comprising” certain steps or features, it is also meant to encompass the same method or composition consisting essentially of those steps or features and consisting of those steps or features.

Each components or composition herein described is useful in another embodiment or in any method described herein. It is also intended that each component or compositions herein described as useful in the methods, is itself an embodiment of the invention.

### II. Cell Perturbations and Sample Preparation

In certain embodiments, prior to the tagmentation/chromatin accessibility steps of the method, cells and cell nuclei samples are prepared. In certain embodiments, herein, the cell is a eukaryotic cell such as a plant cell, an animal cell, a fungal cell, a protozoa cell or an algae cell. In one embodiment, the cell is a mammalian cell. In a further embodiment, the cell is a stem cell (for example, an embryonic stem cell), a cancer cell, a neuronal cell, an epithelial cell (for example, a lymphocyte), an immune cell, an endocrine cell, a germ cell, a somatic cell, a kidney cell, a liver cell, a pancreatic cell, a skin cell, a fat cell, a bone cell, and a muscle cell. In one embodiment, the cell is from a cell line, for example, a HEK293 cell, a NIH-3T3 cell, or a K562 cell.

The method described herein may apply to cells that are perturbed, for example, by a gain-of-function genomic editing, a loss-of-function genomic editing, an upregulation or downregulation of certain coding or non-coding genomic sequence, or epigenome editing. Such perturbation may be achieved via one or more of electroporation, calcium phosphate precipitation, microinjection, transformation, viral infection, transfection, liposome delivery, membrane fusion techniques, high velocity DNA-coated pellets, viral infection and protoplast fusion, RNA interference (RNAi), and CRISPR-Cas.

In certain embodiments, the perturbation involves culturing the cells with a chemical agent or a biological agent or actively physically disturbing the cell culture. The term chemical agent includes various small molecule drugs/compounds, while the term biological agent refers to biological drugs, which are a diverse category of drugs and are generally large, complex molecules. These biological drugs may be produced through biotechnology in a living system, such as a microorganism, plant cell, or animal cell. Types of biological products approved for use in the United States, including therapeutic proteins (such as filgrastim), monoclonal antibodies (such as adalimumab), vaccines (such as those for influenza and tetanus), cell therapy drug (for example, CarT), and gene therapy drug (for example, recombinant AAV vectors). During the perturbation step, the cells may be incubated with the chemical and/or biological agent or any combinations thereof, such as a library of peptides or a library of small molecules or a library of anti-cancer drugs, which are available commercially or publicly. See, for example, www.selleckchem.com/screening/anti-cancer-compound-library.html?gclid=CjwKCAjw0tHoBRBhEiwAvP1GFfLrUWZGJpXyE_QMr_f3NMvn9tC8433K8edIeOYkL08wUNdHzzwgFhoCquQQAvD_BwE, www.genscript.com/peptide-library.html, www.creative-biolabs.com/drug-discovery/therapeutics/whole-peptide-library.htm, phoenixpeptide.com/products/category/Peptide-Libraries/, www.selleckchem.com/screening/express-pick-library-premium-version.html?gclid=CjwKCAjw0tHoBRBhEiwAvP1GFTm7F6ezXNk1pUNajAWqP8Nc4C Oj2N1MNTes9pEGADe8nMF7UmUgPxoCT9cQAvD_BwE, www.selleckchem.com/screening/fda-approved-drug-library.html and www.chembridge.com/screening_libraries/. In certain embodiments, the cells are contacted with various chemical drugs or biological drugs for large-scale drug screens. In certain embodiments, the cells are treated via CRISPR-Cas enzyme and various guide RNA. The term physical disturbance refers to an active mixing, shaking, stretching, or stirring of the cells in culture. In certain embodiments, a population of cells is treated separately with any one of the perturbations as described herein or with any combinations of the perturbations, resulting in a heterologous population of cells.

As used herein, the term “a heterologous population of cells” refers to multiple cells, which are not identical to each other. In another example for heterologous population of cells, a subset of cells (i.e., part of but not the whole cell population) may be treated with each drug of the drug libraries as described above separately. Such cells may be barcoded and processed in the method(s) as described herein. In yet another example, the cells are perturbated via CRISPR-Cas using a vector library as described herein. After this perturbation, a different vector may be introduced into the cells which leads to a heterologous population.

As used herein, downregulation is a perturbation process by which a cell decreases the quantity of a cellular component, such as a genomic sequence or its corresponding RNA or protein, in response to a perturbation, by at least about 10%, about 20%, about 30%, about 40%, about 50%, about 60%, about 70%, about 80%, about 90%, about 95% compared to a control cell without the perturbation. The complementary process that involves increases of such components in response to a perturbation, by at least about 10%, about 20%, about 30%, about 40%, about 50%, about 60%, about 70%, about 80%, about 90%, about 95%, about 1 fold, about 2 fold, about 5 fold, about 10 fold, about 50 fold, about 100 fold or more compared to a control cell without the perturbation is called upregulation.

In certain embodiments, the method(s) described herein comprises a perturbation step comprising transducing the cells with one or more vectors and culturing the cells. Each vector comprises a nucleic acid sequence encoding a Cas protein in operative association with a first promoter which controls expression of the Cas protein, and a CRISPR guide RNA coding sequence in operative association with a second promoter which controls transcription thereof. In certain embodiments, the RNA in the reverse transcription step comprises the guide RNAs. In certain embodiments, the cells are incubated with the vector at a multiplicity of infection (MOI) of about 0.05, about 0.1, about 0.2, or about 0.3. In certain embodiments, the vector is a lentiviral vector.

In a further embodiment, the first promoter is an inducible promoter, such as a doxycycline inducible promoter. In a preferred embodiment, the first promoter is an RNA pol II promoter. A RNA pol II promoter is a promoter that is sufficient to direct accurate initiation of transcription by the RNA polymerase II machinery, wherein the RNA polymerase II (RNAP II and Pol II) is a RNA polymerase found in the nucleus of eukaryotic cells, catalyzing the transcription of DNA to synthesize precursors of messenger RNA (mRNA) and most small nuclear RNA (snRNA) and microRNA.

A variety of Polymerase II promoters that can be used within the compositions and methods described herein are publicly or commercially available to a skilled artisan, for example, viral promoters obtained from the genomes of viruses including promoters from polyoma virus, fowlpox virus (UK 2,211,504), adenovirus (such as Adenovirus 2 or 5), herpes simplex virus (thymidine kinase promoter), bovine papilloma virus, avian sarcoma virus, cytomegalovirus (CMV), a retrovirus (e.g., MoMLV, or RSV LTR), Hepatitis-B virus, Myeloproliferative sarcoma virus promoter (MPSV), VISNA, and Simian Virus 40 (SV40); other heterologous mammalian promoters including the actin promoter, β-actin promoter, immunoglobulin promoter, heat-shock protein promoters, human Ubiquitin-C promoter, PGK promoter. Additional promoters are readily known and available. See, e.g., (Kadonaga, 2012), WO 2014/15134, and WO 2016/054153. In one particular embodiment, the promoter is a CMV promoter.

In one embodiment, the second promoter is an RNA pol III promoter. As recognized by one of skill in the art, a RNA pol III promoter is a promoter that is sufficient to direct accurate initiation of transcription by the RNA polymerase III machinery, wherein the RNA polymerase III (RNAP III and Pol III) is a RNA polymerase transcribing DNA to synthesize ribosomal 5S ribosomal RNA (rRNA), transfer RNA (tRNA), crRNA, and other small RNAs (for example, guide RNA). A variety of Polymerase III promoters which can be used with the invention are publicly or commercially available, for example the U6 promoter, the promoter fragments derived from H1 RNA genes or U6 snRNA genes of human or mouse origin or from any other species. In addition, pol III promoters can be modified/engineered to incorporate other desirable properties such as the ability to be induced by small chemical molecules, either ubiquitously or in a tissue-specific manner. For example, in one embodiment the promoter may be activated by tetracycline. In another embodiment, the promoter may be activated by IPTG (lad system). See, U.S. Pat. Nos. 5,902,880A and 7,195,916B2. In another embodiment, a Pol III promoter from various species might be utilized, such as human, mouse or rat.

In one embodiment, more than one (i.e., multiple) CRISPR guide RNA transcribed from the vectors is targeted to each functional unit of a cell genome of interest. In certain embodiments, there are about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 50, about 75, about 100 or more different guide RNAs targeted to each functional unit of a cell genome of interest. In certain embodiments, each vector transcribes a single guide RNA. In certain embodiments, each vector transcribes about 2, about 3, about 4, about 5, about 6, about 7, about 8, about 9, about 10, about 15, about 20, about 25, or more guide RNAs.

As used herein, the functional unit of a cell genome of interest refers to a genomic sequence which serves a certain function or is suspected of having a certain function. Such function may be expressing a protein of interest, transcribing to an RNA of interest, or regulating a gene of interest. A functional unit of a cell genome typically encompasses a limited region of the genome, such as a region of 1, 5, 10, 20, 30, 40, 50, 60, 70, 80, 90 to 100 kb of genomic DNA. In one embodiment, the functional unit of a cell genome is a coding sequence. In certain embodiments, the functional unit of a cell genome is a non-coding genomic sequence. In a further embodiment, the non-coding sequence may be in regions 5′ and 3′ of the coding region of a gene of interest.

In still other embodiments, the method described herein comprises a preparation step, in which the cells are lysed in a resuspension buffer. In certain embodiments, the cell membrane is lysed but the cell nuclei remain intact. In certain embodiments, the lysed cells still contain mitochondria. For example, using the cell lysing method performed in the Examples, an about 20% to about 50% mitochondrial reads were found in the ATAC library. Therefore, as used herein, the term “cell nucleus” or any grammatical variation thereof may refer to a cell nucleus, the membrane-bound organelle found in eukaryotic cells which contains cell genome. It may also include some cytosomal/cytosomic components which remain physically attached to the cell nucleus after cell lysing, for example, endoplasmic reticulum (ER) connected to the nucleus and some mitochondria.

In certain embodiments, the preparation step is performed after the perturbation step and before the tagmentation step. In one embodiment, the resuspension buffer (i.e., cell lysing buffer) comprises Tween-20 and Igepal CA630. In one embodiment, the cell lysing buffer comprises about 0.01% to about 1% Tween-20. In another embodiment, the cell lysing buffer comprises about 0.01% to about 1% of Igepal CA630. In still another embodiment, the cell lysing buffer comprises about 0.1% Tween-20 and about 0.1% Igepal CA630. In certain embodiments, part of the cytoplasm is retained since the lysis is gentle, which allows detection and analysis of mitochondrial DNA or RNA or any DNA or RNA in the retained cytoplasm.

In certain embodiments, the preparation step also comprises fixing the cells before lysis and optionally washing the fixed cells. In certain embodiments, the cells are fixed via suspension in a fixation buffer. In certain embodiments, the fixation buffer comprises glyoxal. Additionally, or alternatively, the fixation buffer comprises ethanol. In certain embodiments, the fixation buffer comprises about 5% to 30% (v/v) ethanol and about 1% to about 5% (v) glyoxal. In certain embodiments, the fixation buffer comprises about 20% (v/v) ethanol and about 3.1% (v/v) glyoxal at a pH of about 5.0. In a further embodiment, the fixation buffer is made by mixing 280 parts of H₂O, 79 parts of 100% ethanol, 31 parts of 40% glyoxal, and 3 parts of glacial acetic acid, and adjusting pH to about 5.0 and the final volume to about 400 parts using NaOH. As used herein, “v/v” indicates a volume ration while parts are measured in volume as well. For example, x % (v/v) of glyoxal indicates x ml of glyoxal in a final volume of 100 ml. In certain embodiments, the cells are fixed for about 5, about 7, about 10, about 30, about 60 minutes at room temperature. It was found that glyoxal fixation resulted in better preservation of intact nuclei than the more commonly used paraformaldehyde fixative.

### III. Chromatin Accessibility/Tagmentation

Chromatin accessibility is the degree to which nuclear macromolecules are able to physically contact chromatinized DNA and is determined by the occupancy and topological organization of nucleosomes as well as other chromatin-binding factors that occlude access to DNA. If such physical contact can be established in a certain region of the DNA, that DNA region is considered to be in an open chromatin state. The organization of accessible chromatin across the genome reflects a network of permissible physical interactions through which enhancers, promoters, insulators, and chromatin-binding factors cooperatively regulate gene expression. This landscape of accessibility changes dynamically in response to both external stimuli and developmental cues, and emerging evidence suggests that homeostatic maintenance of accessibility is itself dynamically regulated through a competitive interplay between chromatin-binding factors and nucleosomes. See, for example, Klemm et al., Chromatin accessibility and the regulatory epigenome. Nat Rev Genet. 2019 April; 20(4):207-220. doi: 10.1038/s41576-018-0089-8, which is incorporated herein by reference. Therefore, it is important to illustrate how chromatin accessibility defines regulatory elements within the genome and how these epigenetic features are dynamically established to control gene expression. As used herein, the term “chromatin accessibility” may refer to chromatin accessibility across the cell genome.

Current chromatin accessibility assays are used to separate the genome by enzymatic or chemical means and isolate either the accessible or protected locations. The isolated DNA is then quantified using a next-generation sequencing platform. As further shown in the Examples, ATAC-seq (Assay for Transposase-Accessible Chromatin using sequencing) is a technique used in molecular biology to assess genome-wide chromatin accessibility. Specifically, ATAC-seq identifies accessible DNA regions by probing open chromatin with a transposase (for example, a hyperactive mutant Tn5 transposase) that inserts sequencing adapters into open regions of the genome. The transposase excises any sufficiently long DNA in a process called tagmentation: the simultaneous fragmentation and tagging of DNA performed by transposase pre-loaded with sequencing adaptors. The tagged DNA fragments (referred to as fragmented DNA or tagmented DNA) are then purified, amplified by PCR and sent for sequencing. Sequencing reads can then be used to infer regions of increased accessibility as well as to map regions of transcription-factor binding sites and nucleosome positions.

Other available methods for identifying open chromatin regions include, but are not limited to, MNase-seq (Micrococcal nuclease-assisted isolation of nucleosomes sequencing which sequences micrococcal nuclease sensitive sites), FAIRE (Formaldehyde-Assisted Isolation of Regulatory Elements)-seq (which is based on the fact that the formaldehyde cross-linking is more efficient in nucleosome-bound DNA than it is in nucleosome-depleted regions of the genome) and DNAse-seq (DNase I hypersensitive sites sequencing, which is based on the genome-wide sequencing of regions sensitive to cleavage by DNase I).

In the tagmentation step of this method, cell nuclei, each of which comprises DNAs and RNAs from one cell, are obtained from lysed or otherwise perturbed cells and incubated with a transposome complex in a tagmentation buffer. The transposome complex comprises a transposase, a transposon, and a first barcode. The first barcode is ligated to double-stranded DNA at a staggered break caused/produced by the transposase.

A “transposase” is an enzyme that binds to the end of a transposon and catalyzes its movement to another part of the genome by a cut and paste mechanism or a replicative transposition mechanism. In one embodiment, such enzyme is a member of the RNase superfamily of proteins which includes retroviral integrases. Examples of transposases include Tn3, Tn5, and hyperactive mutants thereof. Tn5 can be found in Shewanella and Escherichia bacteria. An example of a hyperactive mutant Tn5 comprises a mutation of E54K. In certain embodiments of this method, the transposase is TnY or Tn5.

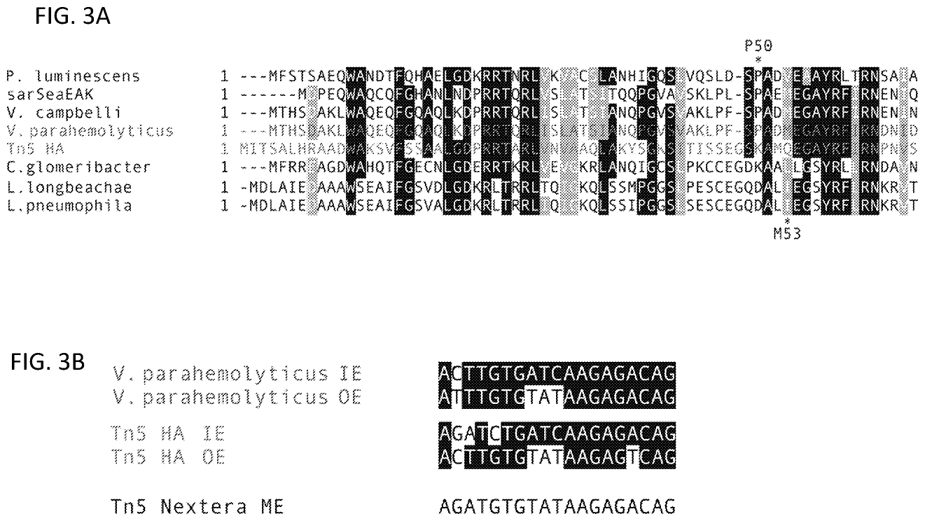

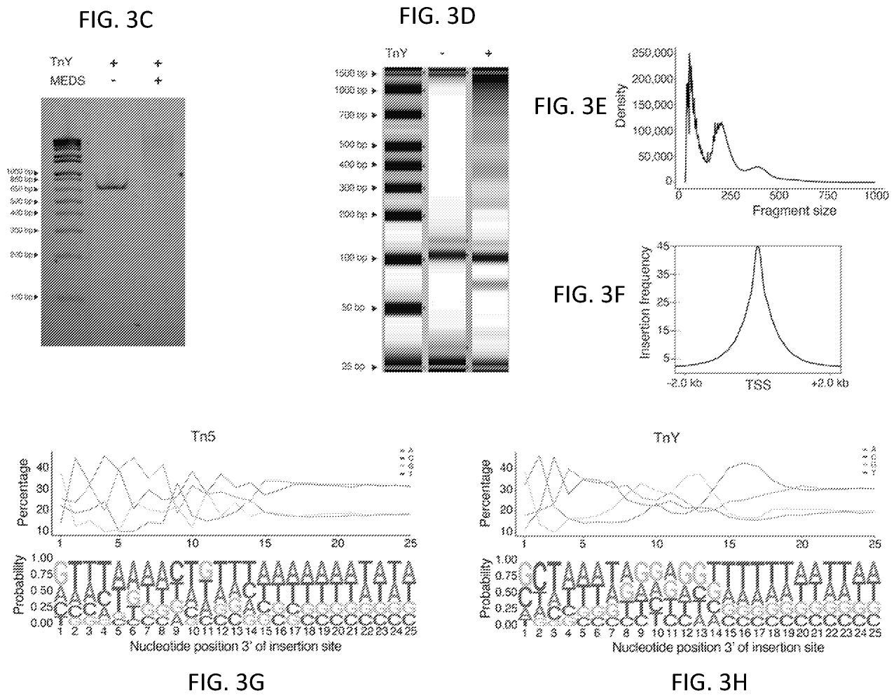

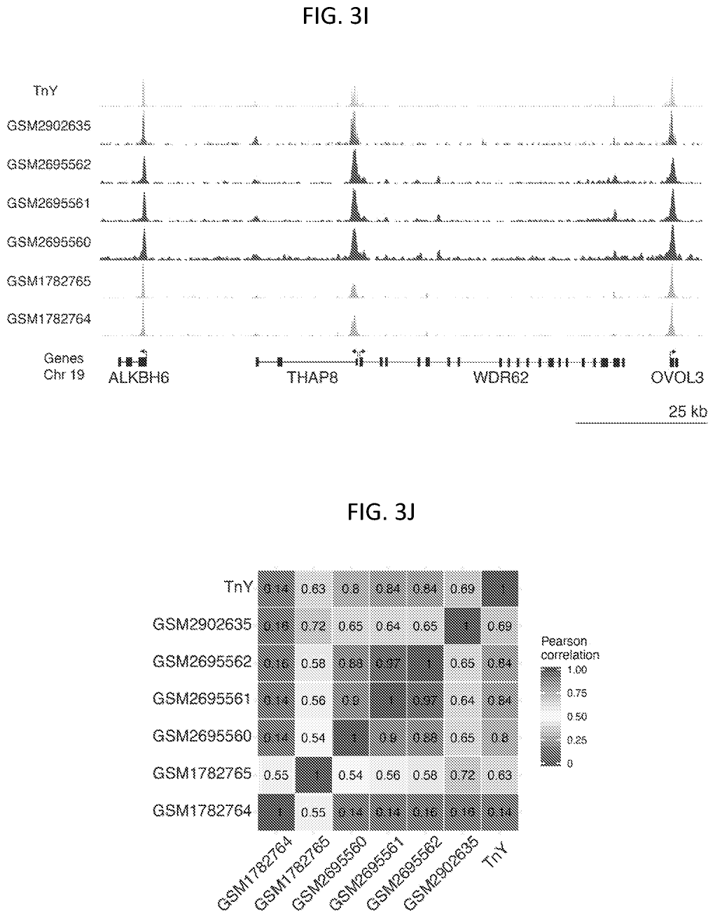

In certain embodiments, the transposase is TnY. TnY is a hyperactive mutant of the transposase from Vibrio parahemolyncus (ViPar). The inside and outside ends (IE and OE, respectively) of the ViPar transposon utilize the same sequence as the IE and OE of the Tn5 transposon, suggesting the ViPar transposon would be compatible with existing Tn5-based workflows (FIG. 3A and FIG. 3B). Two mutations were introduced: (1) P50K, equivalent to the mutation E54K in Tn5, which is predicted to make the transposon hyperactive³¹ and (2) M53Q, which changes the residue that interacts with nucleotide 9 (a thymine) on the non-transferred strand of the mosaic end (ME) similar to Tn5 Q57, predicted to increase binding to the Tn5 ME. The ViPar transposase with P50K and M53Q mutations, henceforth referred to as TnY, showed Tn5 ME loading and tagmentation activity (FIG. 3C-FIG. 3F). Finally, the insertion site preference of TnY was characterized by performing tagmentation on NA12878 DNA and sequencing on a MiSeq Instrument (Illumina); it was found that TnY has insertion site preferences distinct from, but of a similar magnitude to those of Tn5 (FIG. 3G and FIG. 3H).

As used herein, the term “transposon” is used interchangeably with sequencing adapter, referring to a nucleic acid molecule that is capable of being incorporated into a nucleic acid by a transposase enzyme. A transposon includes two transposon ends (also termed “arms” and “mosaic end” or “ME”, for example, a double-stranded mosaic end comprising a pMENT common oligo as used in the Examples). In one embodiment, the two transposon ends are linked by a sequence that is sufficiently long to form a loop in the presence of a transposase. Transposons can be double-, single-stranded, or mixed, containing single- and double-stranded region(s), depending on the transposase used to insert the transposon. For Mu, Tn3, Tn5, Tn7, or TnlO transposases, the transposon ends are double-stranded, but the linking sequence need not be double-stranded. In a transposition event, these transposons are inserted into double-stranded DNA. The term “transposon end” refers to the sequence region that interacts with transposase. The transposon ends are double-stranded for transposases Mu, Tn3, Tn5, Tn7, TnlO, etc. The transposon ends are single-stranded for transposases IS200/IS605 and ISrad2, but form a secondary structure, just like a double-stranded region. Examples of transposon end sequences can be found in FIG. 3B. In a transposition event, single-stranded transposons are inserted into single-stranded DNA by a transposase enzyme. See, for example, US20150337298A1, which is incorporated herein by reference.

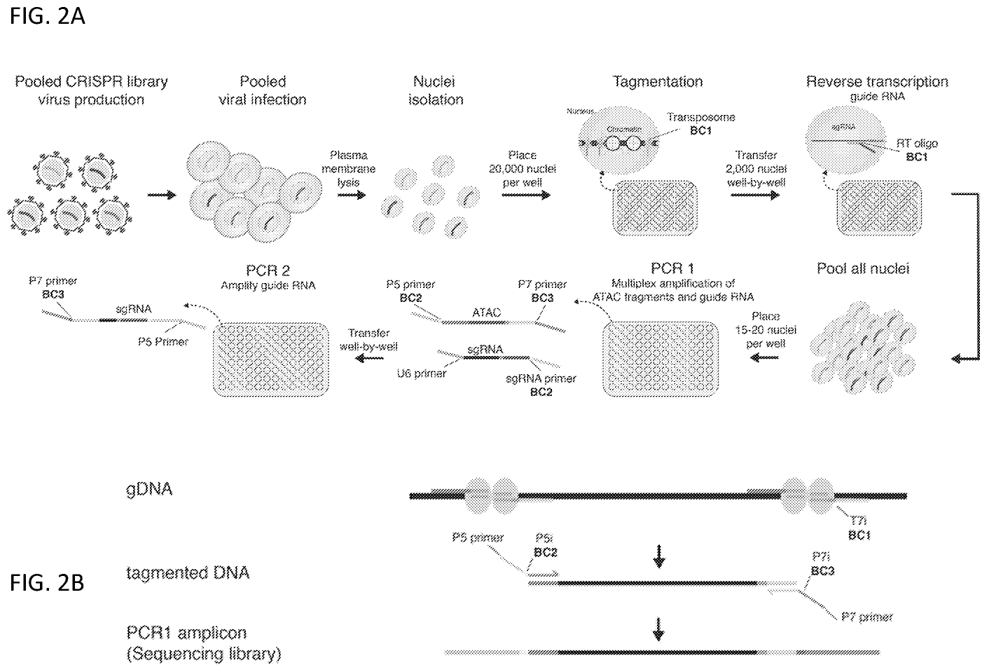

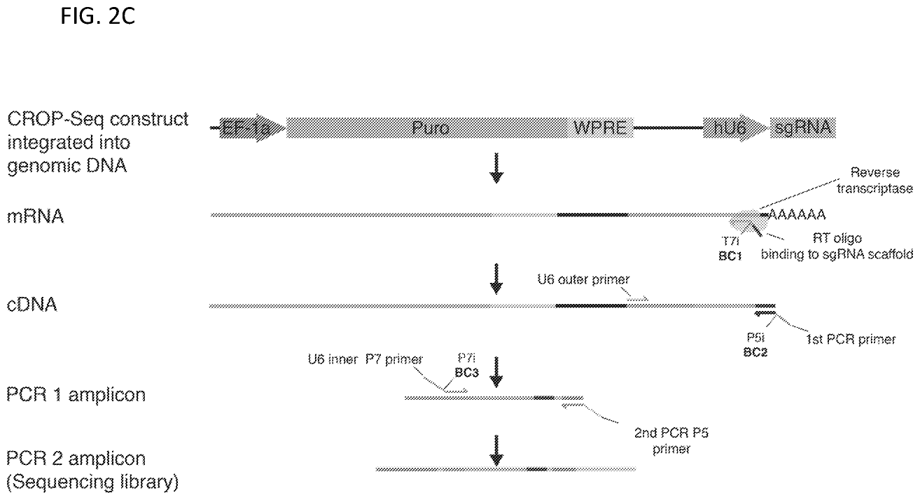

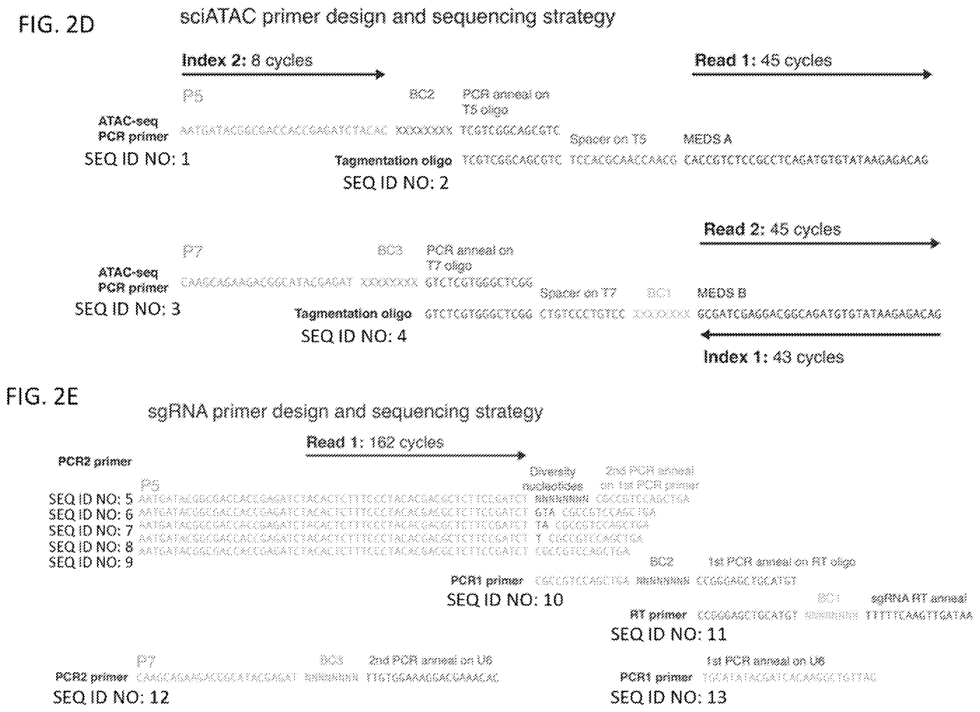

In one embodiment, the transposome complex comprises a transposase assembled with a transposon comprising two mosaic end double-stranded (MEDS) oligos. In a further embodiment, the transposome complex further comprises a barcode in one or both of the MEDS oligos. In certain embodiments, the transposome complex further comprises a nucleic acid sequence at the 5′ ends of the MEDS oligos, wherein the nucleic acid sequence is able to anneal to a PCR primer. For example, a T5 oligo may be annealed to MEDS A and a T7 oligo may be annealed to MEDS B as illustrated in FIG. 2B-FIG. 2E.

As used herein, a barcode describes a defined polymer, e.g., a polynucleotide, which when it is a functional element of the polymer construct, is specific for a compartment, a single cell, or cell nucleus or cellular components (for example, DNA, RNA and/or mitochondria and ribosomes) thereof. In one embodiment, the barcode is about 2 to 4 monomeric components, e.g., nucleotide bases, in length. In other embodiments, the barcode is at least about 1 to 100 monomeric components, e.g., nucleotides, in length. Thus, in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99, or up to 100 monomeric components, e.g., nucleic acids. A barcode can be an artificial sequence or a naturally occurring sequence. In certain embodiments, each barcode within a population of barcodes is different. In other embodiments, a portion of barcodes in a population of barcodes is different, e.g, at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error correcting barcodes. Barcodes can be used to computationally deconvolute the multiplexed sequencing data and identify sequence reads derived from an individual cell, compartment, etc. A barcode can also be used for deconvolution of a collection of cells or cell nuclei or cellular components thereof that have been distributed into small compartments for enhanced mapping.

In certain embodiments, the term “barcode” also refers to a process of introducing a barcode to a DNA or RNA. Examples of introducing a barcode are illustrated in FIG. 2B—FIG. 2E. In one embodiment, a barcode may be located at the 3′ end of a reverse transcription (RT) primer, such as, a RT primer comprising a oligo d(T)n (also termed as RT oligo, referring to a polyT oligo) at the 5′ end and a barcode at the 3′ end. In certain embodiments, a barcode may be located at the 3′ end of a PCR primer. Such primer may be used in amplifying tagmented DNA or cDNA via a PCR reaction.

In certain embodiments, each polymer (such as DNA or RNA) may be barcoded using a “unique molecular identifier” (UMI), also called equivalently a “random molecular tag” (RMT), which is a random sequence of monomeric components of a polymer as described above, e.g., nucleotide bases, is specific for that polymer. The UMI permits identification of amplification duplicates of the polymer with which it is associated. In the description of the methods and compositions herein, one or more UMI may be associated with a single polymer. The UMI may be positioned 5′ or 3′ to the barcode in the composition. In another embodiment, the UMI may be inserted into the polymer as part of the described methods. In one embodiment of the methods described herein, a UMI is added during the method, for example, during reverse transcription. Each UMI for each polymer e.g., oligonucleotide or polynucleotide, is different from any other UMI used in the compositions or methods. In any embodiment, the UMI is formed of a random sequence of DNA, RNA, modified bases or combinations of these bases or other monomers of the polymers identified above. In one embodiment, a UMI is about 8 monomeric components, e.g., nucleotides, in length. In other embodiments, each UMI can be at least about 1 to 100 monomeric components, e.g., nucleotides, in length. Thus, in various embodiments, the UMI is formed of a random sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

As used herein, the term “compartment” refers to a physical area or volume that separates or isolates a subset of cell nuclei/cells/cellular components from other subsets. In one embodiment, a subset may be a single cell nucleus or cell or cellular components from a single cell, and the compartment isolates each cell nucleus or cell or cellular components thereof. In another embodiment, the subset may contain nₙ or mₙ of cell nuclei or cell or cellular components thereof. A compartment may be an aqueous compartment (for example, microfluidic droplet), a solid compartment (for example, a well on a plate, a tube, a vial, a particle, a microparticle, and/or a bead), or a separated region on a surface (for example, a chip, a microplate, or a slide).

For use in the tagmentation step of the method, in one embodiment, the tagmentation buffer comprises H₂O, 5 mM Mg²⁺, a hydrophilic solvent in a zwitterionic buffer at a pH of about 8.5. In certain embodiments, the tagmentation buffer comprises a transposome complex. In a further embodiment, the zwitterionic buffer is TAPS-NaOH. In yet a further embodiment, the tagmentation buffer comprises a RNase inhibitor. In certain embodiments, the tagmentation buffer is 10 mM TAPS-NaOH at pH 8.5, 5 mM MgCl₂, 10% DMF and RNase inhibitor. In a further embodiment, the RNase inhibitor is a RIBOLOCK RNase inhibitor.

In certain embodiments, the transposome complex and the cell nuclei are incubated for 30 minutes at 37° C. in the tagmentation step. In certain embodiments, the tagmentation step further comprises one or both (i) adding EDTA, whereby the tagmentation reaction is stopped, and (ii) quenching the EDTA by adding MgCl₂.

As shown in the examples, the transposome complex may be assembled as indicated below.

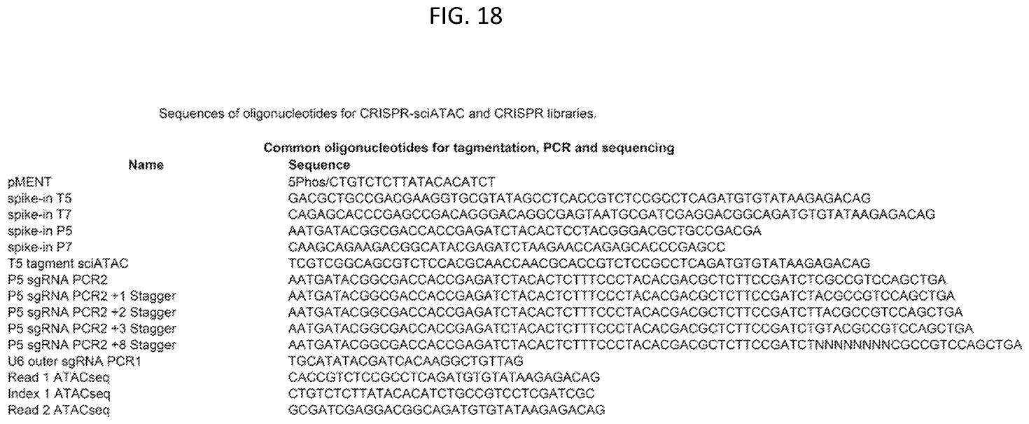

To produce mosaic end double stranded (MEDS) oligos, a single T5 tagmentation oligo can be annealed with the pMENT common oligo (100 μM each) (FIG. 18) as follows in TE buffer: 95° C. for 5 minutes, then cooled at a rate of 0.2° C./s down to 4° C. (“MEDS A”). The same process can be used to anneal each barcoded T7 tagment sciATAC oligo with the pMENT common oligo (“MEDS B”) (FIG. 18). MEDS A and MEDS B are mixed together, diluted 1:6 in TE buffer and 2 μl and transferred into a new tube and mixed with 3 μl of TnY enzyme. After 30 minutes at room temperature to allow for transposome assembly, 45 μl Dilution Buffer is added, mixed by pipetting up and down and stored at −20° C. until ready for tagmentation. Dilution Buffer consists of 2× Dialysis Buffer diluted 1:1 by volume with 100% glycerol.

In certain embodiments, the transposome complex is assembled on the same day as the tagmentation to achieve optimal tagmentation.

### IV. Reverse Transcription

The reverse transcription step allows each of the RNAs (for example, a CRISPR guide RNA, a messenger RNA, a mitochondrial RNA, a microRNA) to be reverse transcribed to a complementary DNA (cDNA) barcoded with the first barcode. In certain embodiments, cell nuclei are incubated with reverse transcription primers barcoded with the first barcode or the corresponding antisense sequence thereof, reverse transcriptase, and dNTPs in a reverse transcription buffer. In certain embodiments, the reverse transcription buffer comprises a RNase inhibitor. In certain embodiments, the RNase inhibitor is a RIBOLOCK RNase inhibitor. In certain embodiments, the first barcode may be unique for each cell. In certain embodiments, the reverse transcriptase is REVERTAID reverse transcriptase. See, for example, www.thermofisher.com/order/catalog/product/EP0442. In certain embodiments, the reverse transcriptase (RT) is another recombinant M-MuLV RT.

As used herein, a barcode unique for each cell/compartment means a barcode sequence in the DNA/RNA from one cell/compartment is different from any other barcode sequences in the DNA/RNA from another cell/compartment.

In certain embodiments, the tagmentation step is performed prior to the reverse transcription step. Without wishing to be bound by theory, the cDNAs are not tagmented via performing the tagmentation step first, thus allowing an easier analysis of chromatin accessibility.

### V. Sequencing and Analysis

During the sequencing step, cell nuclei are digested and DNAs (for example, genomic DNA and/or cDNA) are extracted and sequenced; while the analyzing step provides chromatin accessibility and RNA sequences of each of the cells. In certain embodiments, an optional amplification step is performed before the sequencing step, for example, via increasing copy number of the DNA (including tagmented genomic DNAs as well as cDNAs) via polymerase chain reaction (PCR).

DNA sequencing is the process of determining a nucleic acid sequence—the order of nucleotides in DNA. It includes any method or technology that is used to determine the order of the four bases: adenine, guanine, cytosine, and thymine. Methods of sequencing may include, but do not limited to, Maxam-Gilbert sequencing, shorgun sequencing, bridge PCR, Chain-termination methods, Single-molecule real-time sequencing, Ion semiconductor (Ion Torrent sequencing), Pyrosequencing (454), Sequencing by synthesis (Illumina), Combinatorial probe anchor synthesis (cPAS-BGI/MGI), Sequencing by ligation (SOLiD sequencing), Nanopore Sequencing, Chain termination (Sanger sequencing), Massively parallel signature sequencing (MPSS), and Polony sequencing. Such sequence may be performed on a deep sequencing platform which sequences for multiple times, sometimes hundreds or even thousands of times and/or via a next-generation sequencing (NGS) approach (which is also known as high-throughput sequencing).

After sequencing, the genomic DNAs or cDNAs comprising the same barcode sequence are identified as from the same cell. In certain embodiments, presence of certain RNA in the cell (for example, a microRNA or a CRISPR guide RNA) can be determined through sequencing cDNAs. In a further embodiment, the sgRNA may be aligned, for example, as described in the sgRNA alignment of Example 1. In certain embodiments, transcriptome shown by RNA sequences may be acquired via cDNA sequence, thus providing data available via traditional RNA-seq (RNA sequencing). In certain embodiments, mitochondrial RNAs are acquired.

In certain embodiments, the genomic DNAs (fragmented by transposase in the tagmentation step) are analyzed as in ATAC-seq. For example, sequence reads of the fragmented genomic DNAs are acquired and aligned to a reference genome (for example, using programs available to one of skill in the art such as BWA and Bowtie2). In certain embodiments, one or more parameters for quality control purposes are acquired, for example, fragment size distribution, library complexity, adjusting read start position based on transposase (for example, aligning sequence reads to the positive strand are offset by ±1, 2, 3, 4, 5, 6, 7, 8, 9, 10 bp, and all reads aligning to the negative strand are offset by ±1, 2, 3, 4, 5, 6, 7, 8, 9, 10 bp), and promoter/transcript body score (which is calculated for coverage of promoter divided by the coverage of transcripts body, showing if the signal is enriched in promoters). In one embodiment, aligning sequence reads to the positive strand are offset by +4 bp, and all reads aligning to the negative strand are offset by −5 bp). A summary of the mapping results is provided, separated according to uniqueness and alignment type (concordant, discordant, and non-concordant/non-discordant). Peak-calling identifying enriched (signal) regions in ATAC-seq data is then performed using tools, such as MACS2. In one embodiment, the chromosome position is plotted in x axis and the enrichment score is plotted in y axis. Therefore, peaks in the plot identified enriched regions in chromosome, indicating open chromatin with high chromatin accessibility. One or more of the following may be identified: (1) Nucleosome free, mononucleosome, dinucleosome, and trinucleosome regions; (2) distribution of nucleosome-free and nucleosome-bound regions; (3) transcription factor footprints; (4) sample correlations. Numbers of ATAC fragments, peaks, as well as differential peaks (for example, for comparing ATAC-seq samples from two different conditions) may be obtained using this method.

Examples of procedures can be found in Example 1, including trimming reads with FASTX-Toolkit, demultiplexed using grep (perfect match), alignment demultiplexed based on barcodes, mapping fragments to a reference genome, and peak-calling with MACS2. Additional analysis may include comparing the ATAC-seq peaks to DNaseI hypersensitivity peaks for validation.

In certain embodiments, cells with at least about 50, about 100, about 200, about 300, about 400, about 500, about 600, about 700, about 800, about 900, about 1000, about 2000, about 3000, about 4000, about 5000, about 6000, about 7000, about 8000, or about 9000 unique ATAC-seq fragments are selected for analysis. Additionally or alternatively, each cell is required to have at least about 10, about 20, about 30, about 40, about 50, about 60, about 70, about 80, about 90, about 100, about 200, about 300, about 400, about 500, about 600, about 700, about 800, about 900, about 1000, about 2000, about 3000, or about 4000 RNA (for example guide RNA or microRNA) reads with at least about 90%, about 95%, about 96%, about 97%, about 98%, or about 99% of the reads assigned to one RNA sequence. In certain embodiments, cells with at least about 2000 unique ATAC-seq fragments are selected for analyses. Additionally or alternatively, each cell is required to have at least about 100 guide RNA reads with at least about 99% of the reads assigned to one RNA sequence.

In one embodiment, essential genes are identified via a CRISPR perturbation, for example via identifying loss of guide RNAs targeting an essential gene upon cell culture. For example, probability for loss-of-function intolerance (pLI) scores may be assessed.

In a further embodiment, ChIP-seq may be used to identify enrichment or depletion in accessibility of transcription factor (TF) binding sites following chromatin modifier knock-out. In another embodiment, JASPAR motifs may be used to predict TF binding sites from the JASPAR database was also utilized (386 motifs from JASPAR 2016, human CORE dataset). Transcription factor motif enrichment and depletion scores may be calculated, for example, using chromVAR20. In yet another embodiment, coverage per base around AP-1 motifs using mononucleosomal fragments (defined as paired-end ATAC-seq fragments with a length between 180 and 247 nt9) was calculated, for example, using BEDTools. In one embodiment, accessibility of enhancers and promoters may be determined.

In certain embodiments, a null peak distribution derived from non-perturbated cells is used as a reference and data acquired from perturbated cells is compared to the reference. In certain embodiments, to avoid biases that may arise when comparing coverage between different gene-KOs with different numbers of single cells, each cell population per perturbation is down-sampled to a smaller cell number and the data acquired is compared to a non-perturbated cell population of a similar size. Each population of cells is resampled about 100, about 200, about 500, about 600, about 700, about 800, about 900, about 1000, about 1500, about 2000, about 3000, about 5000, or more times and the coverage at transcription start sites, weak enhancers (midpoint), and strong enhancers (midpoint) is calculated.

### VI. Cellular Indexing and Barcodes

In a further embodiment, the method described comprises performing combinatorial cellular indexing. In certain embodiments, the method comprises transferring the cell nuclei to a first set of compartments prior to the tagmentation step; transferring the cell nuclei to a second set of compartments after the reverse transcription step and prior to the sequencing step; and barcoding each of the DNAs with a second barcode. In this method, cell nuclei from the same first-set compartment are transferred to different second-set compartments, whereby sequences acquired and analyzed with the same combination of the first and the second barcodes are identified as being from the same cell. In certain embodiments, the first barcode is unique for each first-set compartment. In certain embodiments, the second barcode is unique for each second-set compartment. A total of n_{c} first-set compartments contain about nₙ nuclei per compartment, and a total of m_{c} second-set compartments contain about mₙ nuclei per compartment. In certain embodiments, the method further comprises pooling the cell nuclei and randomly distributing the pooled cell nuclei into the second set of compartments, wherein nₙ>>m_{n.}

In one embodiment, the first barcode is unique for each cell. DNA sequences acquired and analyzed with the same first barcode are identified as being from the same cell. In another embodiment, a combinatorial cellular indexing is performed, which comprises transferring the cell nuclei to a first set of compartments prior to the tagmentation step, wherein a total of n_{c} first-set compartments contain about nₙ nuclei per compartment; (ii) transferring the cell nuclei to a second set of compartments after the step of (b) and prior to the step of (c), wherein a total of m_{c} second-set compartments contain about mₙ nuclei per compartment, and (iii) barcoding each of the DNAs with a second barcode. In one embodiment, the first barcode is unique for each first-set compartment, and the second barcode is unique for each second-set compartment. In certain embodiments, cell nuclei from the same first-set compartment are transferred to different second-set compartments, whereby sequences acquired and analyzed with the same combination of the first and the second barcodes are identified as being from the same cell. In one embodiment, the method further comprises pooling the cell nuclei before the sequencing step and randomly distributing the pooled cell nuclei into the second set of compartments. In one embodiment, nₙ>>mₙ. In a further embodiment, nₙ>100×mₙ. In yet a further embodiment, n_{c}=96, nₙ=˜2000, m_{c}=96 to 1152 (including 96 or 1152), mₙ=15 to 20.

As used herein, >> refers to that the first number before >> is larger than the second number after it by 10 fold, 20 fold, 50 fold, 100 fold, 200 fold, 500 fold, or 1000 fold.

In combinatorial indexing, a combination of different barcodes can serve as a single barcode for identification purposes. For ease of discussion, the phrase “a first barcode comprising a n^{th} barcode” is used to describe such combinations. As one example, a first barcode can comprise a third barcode to be ligased to the 5′ terminal of the DNA/RNA and a fourth barcode to be ligased to the 3′ terminal of the DNA/RNA. Additionally, or alternatively, the second barcode comprises a fifth barcode at the 5′ terminal of the DNA and a sixth barcode at the 3′ terminal of the DNA. In this case, to distinguish a number of cells from each other using those barcodes, less barcodes are needed. For example, a total of 20 barcodes with 12 third barcodes and 8 fourth barcodes can generate 96 different combinations (i.e., 96 different first barcodes) for distinguishing 96 cells or 96 compartments.

As shown in the Examples, the combinatorial indexing method directly captures the gRNA (thus captures its targeting sequence) without the need to clone a barcode together with each of the sgRNAs and without the need to use a targeting-sequence-specific PCR primer. The described method, therefore, allows for easy design and scalability of CRISPR pool screens.

### VII. Specific Embodiment of the Methods

In one embodiment, provided herein is an in vitro method for analyzing chromatin accessibility and RNA of each single cell in a library of cells, comprising: (a) incubating cell nuclei in a suspension obtained from lysed cells with a tagmentation buffer that comprises a transposome complex, wherein each cell nucleus comprises DNAs and RNAs from one cell, wherein the transposome complex comprises a transposase, a transposon and a first barcode, wherein the transposase causes staggered double-stranded breaks in the DNAs, and wherein the first barcode is ligased to the double-stranded DNA at the staggered break; (b) performing reverse transcription which comprises contacting and incubating the cell nuclei of (a) with reverse transcription primers barcoded with the first barcode or the corresponding antisense sequence thereof, reverse transcriptase, and dNTPs in a reverse transcription buffer, whereby each of the RNAs is reverse transcribed to a DNA; (c) sequencing DNA, which is extracted from digested cell nuclei of (b); and (d) analyzing chromatin accessibility and RNA of the cells.

As used herein, an antisense sequence corresponding to a barcode is a DNA sequence complementary (i.e., reverse-complement counterpart) to the barcode sequence. In certain embodiments, upon duplicating sequences, the antisense sequence and the corresponding sequence may form a double-strand DNA.

In another embodiment, provided is an in vitro method for analyzing chromatin accessibility and RNA of each single cell in a library of cells, comprising:

(a) a preparation step which comprises (i) lysing the cells to release nuclei therefrom; and (ii) suspending the cell nuclei of (a)(i) in a tagmentation buffer, wherein each cell nucleus comprises DNAs and RNAs from one cell;

(b) a tagmentation step which comprises (i) incubating a transposome complex with the cell nuclei in the tagmentation buffer of (a)(ii), wherein the transposome complex comprises a transposase, a transposon and a first barcode, wherein the transposase causes staggered double-stranded breaks in the DNAs, and wherein the first barcode is ligased to the double-stranded DNA at the staggered break;

(c) a reverse transcription step which comprises (i) contacting and incubating the cell nuclei of (b) with reverse transcription primers barcoded with the first barcode or the corresponding antisense sequence thereof, reverse transcriptase and dNTPs in a reverse transcription buffer, whereby each of the RNAs is reverse transcribed to a DNA; and

(d) a sequencing step which comprises (i) digesting the cell nuclei and extracting DNAs; and (ii) sequencing the DNAs extracted and analyzing chromatin accessibility and RNA of the cells.

In a further embodiment, before the tagmentation step, the cells are lysed individually and the cellular components (including DNA, RNA, and/or mitochondria) from one cell is separated from those of another cell in a compartment, and the tagmentation step, the reverse transcript step as well as the sequence and analyzing step are all performed in the compartment for the cellular components from each cell. In one embodiment, the compartment may be a droplet.

Examples for illustration purposes only can be found in Example 2 with detailed protocols provided in Example 1.

In certain embodiments, the method results in more than 100, 200, 300, 400, 500, 600, 700, 800, 900, 1000, 1100, 1200, 1300, 1400, 1500, or more unique ATAC DNA fragments per cell. Additionally or alternatively, the method result in at least about 10, about 20, about 30, about 40, about 50, about 60, about 70, about 80, about 90, about 100, about 110, about 120, about 130, about 140, about 150, about 200, about 300, about 400, about 500, about 600, about 700, about 800, about 900, about 1000, about 1500, about 2000, or more guide RNA reads.

CRISPR-sciATAC can be applied to study diverse phenotypes and diseases influenced by chromatin accessibility and can be combined with large-scale drug screens of small molecule epigenetic modulators to pinpoint mechanisms of drug action.

### VIII. Compositions and Kits

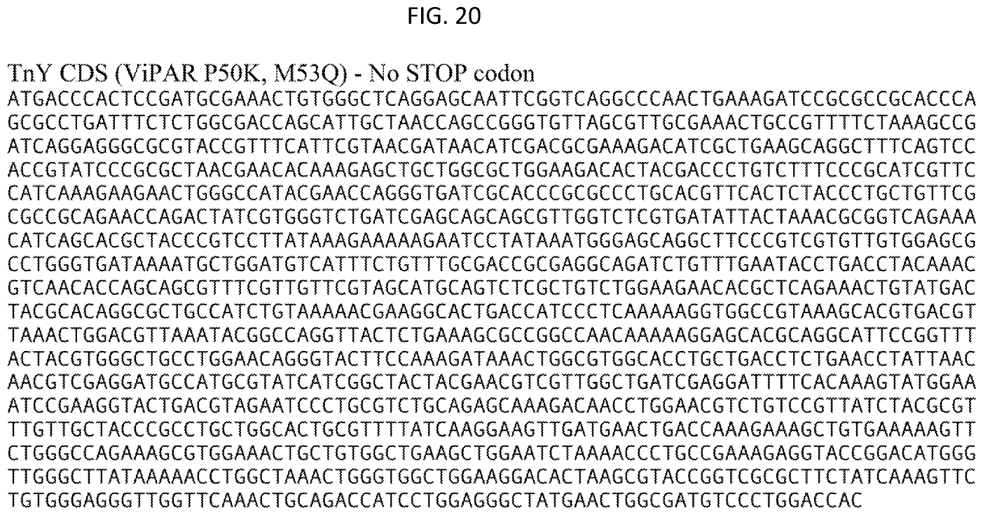

In another aspect, provided are compositions and kits for use in a method as described herein. In one embodiment, provided is a transposase TnY. A nucleic acid sequence for TnY is provided in FIG. 20 and in the sequence listing as SEQ ID NO: 108. Additionally, or alternatively, provided is a cell lysing buffer comprising Tween-20 and Igepal CA630. As shown and discussed in the Examples, such cell lysing buffer helps keep cell nuclei intact after cell lysis. In certain embodiments, the cell lysing buffer comprises 0.1% Tween-20 and 0.1% Igepal CA630. Also, a fixation buffer is provided comprising ethanol and glyoxal. It is found that glyoxal instead of the conventional formaldehyde yields better tagmentation and/or reverse transcription results. In one embodiment, a fixation buffer is provided comprising about 5% to about 30% (v/v) ethanol and about 1% to about 5% (v/v) glyoxal. In certain embodiments, pH of the fixation buffer is about 4.0 to about 7.0, preferably is about 5.0. In another embodiment a fixation buffer comprising about 20% (v/v) ethanol and about 3.1% (v/v) glyoxal at a pH of about 5.0 is provided in the kit. In a further embodiment, the fixation buffer is made by mixing 280 parts of H₂O, 79 parts of 100% ethanol, 31 parts of 40% glyoxal, and 3 parts of glacial acetic acid, and adjusting pH to about 5.0 and the final volume to about 400 parts using NaOH.

In yet another aspect, provided is a kit comprising one or more of the following: a cell lysing buffer, a tagmentation buffer, a transposase, first barcodes, reverse transcriptase, dNTPs, reverse transcription primers barcoded with the first barcode or the corresponding antisense sequence thereof, a reverse transcription buffer, a cell nuclei digestion buffer, and second barcodes. In certain embodiments, the kit further comprises a vector library. In the library, each vector comprises a nucleic acid sequence encoding a Cas protein in operative association with a first promoter which controls expression of the Cas protein, and a CRISPR guide RNA coding sequence in operative association with a second promoter which controls transcription thereof.

### EXAMPLES

The following examples disclose scalable pooled CRISPR screens with single cell chromatin accessibility profiling. A scalable, cost-effective method is provided that combines CRISPR perturbations with a single-cell indexing assay for transposase-accessible chromatin (CRISPR-sciATAC). This method links genome-wide chromatin accessibility to genetic perturbations through simultaneous capture of ATAC-seq fragments and CRISPR guide RNAs from single cells. As described below, a species-mixing experiment showed that CRISPR-sciATAC results in a low doublet rate. CRISPR-sciATAC was applied in human myelogenous leukemia cells to target 21 chromatin-related genes that are frequently mutated in cancer and 84 chromatin remodeling complex subunits and cofactors and generated chromatin accessibility data for nearly 30,000 gene-perturbed single cells. We showed that loss of the H3K27 methyltransferase EZH2 leads to a dramatic increase in accessibility at heterochromatic regions known to play a role in embryonic development and increased expression of multiple HOX genes. Targeting chromatin remodelers generally caused distancing of nucleosomes around transcription factor binding sites. Loss of CoREST subunit SFMBT1 resulted in nucleosome expansion around AP-1 binding sites in promoters but not in enhancers. Loss of SWI/SNF subunit ARID1A resulted in a wide disruption in Transcription Factor Binding Site (TFBS) accessibility, loss of accessibility at enhancers, and affected nucleosome positioning at AP-1 transcription factor binding sites. These examples show that the described CRISPR-sciATAC is a high-throughput, high-resolution, and low-cost single-cell method that can be broadly applied to study the role of genetic perturbations on chromatin in normal and disease states.

The examples are provided for purposes of illustration only. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1—Methods

Cell Culture and Monoclonal K562-Cas9 Cell Line

NIH-3T3 and K562 cells were acquired from ATCC (CRL-1658 and CCL-243). HEK293FT cells were acquired from Thermo Fisher (R70007). NIH-3T3 (mouse) and HEK293FT (human) cells were maintained at 37° C. with 5% CO₂ in D10 media: DMEM with high glucose and stabilized L-glutamine (Caisson DML23) supplemented with 10% fetal bovine serum (Thermo Fisher 16000044). K562 cells were maintained at 37° C. with 5% CO₂ in R10 media: RPMI with stabilized L-glutamine (Thermo Fisher 11875119) supplemented with 10% fetal bovine serum.

To generate monoclonal K562 cells expressing Cas9, K562 cells were transduced with lentiCas9-Blast (Addgene 52962) at a multiplicity of infection (MOI) of 0.1 and selected and maintained in R10 with 5 μg/ml blasticidin. Monoclonal K562-Cas9 cells were isolated and expanded through limiting dilution. Expression of Cas9 was confirmed by Western blot using an anti-2A peptide antibody (Millipore Sigma MABS2005).

Lentiviral CRISPR Libraries

To generate NIH-3T3 and HEK293FT cells expressing single guide RNAs (sgRNAs) for the human/mouse experiment, 10 human non-targeting sgRNAs and 10 mouse non-targeting sgRNAs were individually synthesized and cloned into the lentiviral transfer vector CROPseq-Guide-Purol (Addgene 86708). Equal amounts of each sgRNA plasmid were mixed and then, with packaging plasmids pMD2.G (Addgene 12259) and psPAX2 (Addgene 12260), transfected into HEK293FT cells as previously described2. NIH-3T3 and HEK293FT cells were transduced at MOI ˜0.1 and selected and maintained in D10 with 1 μg/ml puromycin.

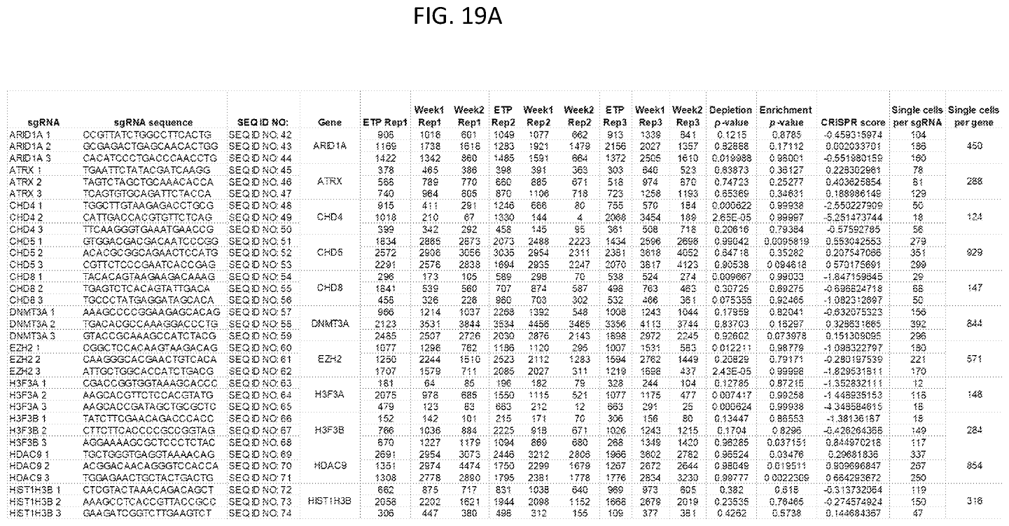

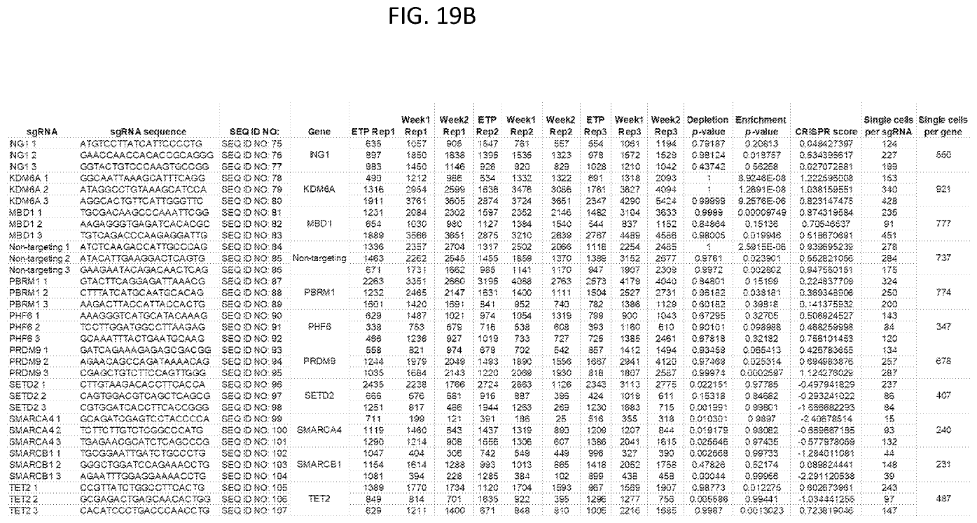

For the chromatin modifier pooled CRISPR screen, 21 frequently mutated chromatin modifiers were identified across all cancers in the Catalogue of Somatic Mutations in Cancer (COSMIC) database⁸ (FIG. 5B) and designed three targeting sgRNAs per gene using the tool GUIDES²⁸. The final library was composed of 63 targeting and 3 non-targeting sgRNAs that were individually synthesized (IDT) and annealed (FIG. 19A and FIG. 19B). Annealed oligos were pooled in equimolar ratio and cloned as a pool into the CROPseq-Guide-Puro lentiviral transfer vector. K562-Cas9 cells were transduced at a MOI of ˜0.1 and selected and maintained in 1 μg/ml puromycin and 5 μg/ml blasticidin. The CRISPR-sciATAC protocol was performed on these cells at week one post-selection.

Transposase Identification and Isolation

A different transposase than Tn5 was used due to the difficulty of obtaining sufficient yields of Tn5 using a previously published Tn5 construct and protocol²⁹. In order to identify new transposases, sequences were aligned using ClustalW³⁰. A range of transposon sequences that were related to the Tn5 sequence were found and a transposon from Vibrio parahemolyticus (ViPar) was selected for further analysis. The inside and outside ends (IE and OE) of the ViPar transposon utilize the same sequence as the IE and OE of the Tn5 transposon, suggesting the ViPar transposon would be compatible with existing Tn5-based workflows (FIGS. 3A and 3B). The identified ViPar transposase was synthesized (Twist BioSciences) and cloned into the vector pTXB1 (NEB, N6707S). Two mutations were introduced: (1) P50K, equivalent to the mutation E54K in Tn5, which is predicted to make the transposon hyperactive³¹ and (2) M53Q, which changes the residue that interacts with nucleotide 9 (a thymine) on the non-transferred strand of the mosaic end (ME) similar to Tn5 Q57, predicted to increase binding to the Tn5 ME. The ViPar transposase with P50K and M53Q mutations, henceforth referred to as TnY, showed Tn5 ME loading and tagmentation activity (FIG. 3C-FIG. 3H). Finally, the insertion site preference of TnY was characterized by performing tagmentation on NA12878 DNA and sequencing on a MiSeq Instrument (Illumina); it was found that TnY has insertion site preferences distinct from, but of a similar magnitude to those of Tn5 (FIG. 3G and FIG. 3H).

Transposase Production

The pTXB1-TnY vector was transformed into BL21(DE3) competent E. coli cells (NEB C2527) and TnY was produced via intein purification with an affinity chitin-binding tag²⁹. One liter of LB culture was grown at 37° C. to OD600=0.6. TnY expression was then induced with IPTG 0.5 mM at 18° C. overnight. After induction, cells were pelleted and then frozen at −80° C. overnight. Cells were then lysed by sonication in 100 ml HEGX (20 mM HEPES-KOH at pH 7.5, 0.8 M NaCl, 1 mM EDTA, 10% glycerol, 0.2% Triton X-100) with a protease inhibitor cocktail (Roche 04693132001). The lysate was pelleted at 30,000×g for 20 min at 4° C. Supernatant was transferred to a new tube, 3 μl of neutralized PEI 8.5% (Sigma Aldrich P3143) was added dropwise to each 100 μl of bacteria extract, gently mixed and centrifuged at 30,000×g for 30 minutes at 4° C. to precipitate DNA. The supernatant was loaded on four 1-ml chitin columns (NEB S6651S). Columns were washed with 10 ml HEGX; 1.5 ml HEGX containing 100 mM DTT was added to the column and incubated for 48 h at 4° C. to allow cleavage of TnY from the intein tag. TnY was eluted directly into two 30 kDa MWCO spin columns (Millipore UFC903008) by adding 2 ml of HEGX. Protein was dialyzed in five dialysis steps using 15 ml 2× Dialysis Buffer (100 HEPES-KOH at pH 7.2, 0.2 M NaCl, 0.2 mM EDTA, 2 mM DTT, 20% glycerol) and concentrated to 1 ml by centrifuging at 5,000×g. The protein concentrate was transferred to a new tube and mixed with an equal volume of glycerol 100%. Then, Triton X-100 was added (0.04% final concentration). TnY aliquots were stored at −80° C.

Transposome Assembly

To produce mosaic end double stranded (MEDS) oligos, we annealed the single T5 tagmentation oligo with the pMENT common oligo (100 μM each) (FIG. 18) as follows in TE buffer: 95° C. for 5 minutes, then cooled at a rate of 0.2° C./s down to 4° C. (“MEDS A”). The same process was used to anneal each barcoded T7 tagment sciATAC oligo with the pMENT common oligo (“MEDS B”) (FIG. 18). MEDS A and MEDS B were mixed together, diluted 1:6 in TE buffer and 2 μl were transferred into a new tube and mixed with 3 μl of TnY enzyme. After 30 minutes at room temperature to allow for transposome assembly, we added 45 μl Dilution Buffer, mixed by pipetting up and down and stored at −20° C. until ready for tagmentation. Dilution Buffer consists of 2× Dialysis Buffer (see Transposase production above) diluted 1:1 by volume with 100% glycerol. We observed optimal tagmentation when transposome assembly was carried out on the same day as the CRISPR-sciATAC tagmentation.

PfuX7 Polymerase Production

The PfuX7 DNA polymerase was produced as previously described³². Briefly, BL21(DE3) competent E. coli cells (NEB C2527) transformed with pETPfuX7 were grown in 1 L of LB culture at 37° C. to OD600=0.6. PfuX7 expression was then induced with IPTG (0.5 mM final concentration) at 30° C. overnight. After induction, cells were pelleted and resuspended in 20 ml Lysis Buffer (50 mM Tris-HCl pH8, 150 mM NaCl, 1 mM EDTA, 1 mM PMSF, 10 μg/ml EDTA-free protease inhibitor (Sigma 11873580001)) and sonicated in an ice slurry. Sonication was at 20% amplitude for ten cycles of 1 minute duration with a 30 second pause between cycles (Branson Ultrasonics, Model 450 Digital Sonifier). The lysate was pelleted at 30,000×g for 15 min at 4° C. Supernatant was transferred to a new tube and incubated with DNA Digestion Buffer (20 μl DNaseI (NEB M0303), 0.5 mM CaCl₂, 2.5 mM MgCl₂) for 30 minutes at 37° C. DNaseI was then inactivated by incubating for 30 minutes at 85° C. After inactivation, the lysate was placed on ice for 20 minutes. Lysate was then centrifuged at 50,000×g for 20 minutes at 4° C. Supernatant was loaded on two 1-ml Ni-NTA (Qiagen 30210) columns, washed twice with Wash Buffer (50 mM Tris-HCl pH 8, 150 mM NaCl). PfuX7 enzyme was eluted in 5 ml Elution Buffer (50 mM Tris-HCl pH 8, 150 mM NaCl, 0.25 M imidazole) and desalted in Storage Buffer (100 mM Tris-HCl pH 8, 0.2 mM EDTA, 2 mM DTT) by performing buffer exchange three times using one Amicon 30 kDa MWCO spin column (Millipore UFC903008). The purified protein was then transferred to a new tube, combined with equal volume of 100% glycerol and adjusted with Tween-20 (0.1% final concentration) and IGEPAL CA630 (0.1% final concentration). Aliquots were stored at −20° C.

Bulk ATAC-Seq

Bulk ATAC-seq experiments were performed as described previously³³. Briefly, 500,000 cells were resuspended in 1 ml PBS and gently lysed by adding 10 ml Resuspension Buffer (10 mM Tris-HCl at pH 7.5, 10 mM NaCl, 3 mM MgCl2) with 0.1% Tween-20. Cells were then centrifuged at 500×g for 10 min at 4° C. to pellet the nuclei. Pelleted nuclei were resuspended in 600 μl 1× Tagmentation Buffer (10 mM TAPS-NaOH at pH 8.5, 5 mM MgCl2, 10% DMF), 30 μl (˜25,000 nuclei) were then transferred into 1.5 ml tubes and 20 μl TnY transposomes were added. Tagmentation was performed at 37° C. for 30 min. Samples were then purified using the DNA Clean & Concentrator kit (Zymo Research D4014) and eluted in 10 μl TE. Eluted DNA was thermocycled with PfuX7 in Phusion GC Buffer (Thermo Fisher F519L) as follows: 72° C. 5 min, 98° C. 30 s, (98° C. 10 s, 63° C. 30 s, 72° C. 3 min)×10 cycles, 4° C. hold. Samples were purified using the DNA Clean & Concentrator kit, eluted in 6 μl TE and size-selected using a 0.9× volume of Ampure XP Beads (Beckman Coulter A63882) to remove excess oligos.

CRISPR-sciATAC: Human and Mouse Cell Mixing Experiment

HEK293FT (human) and NIH-3T3 (mouse) transduced with non-targeting sgRNAs libraries were grown separately. On the day of the experiment, cells were counted, and 500,000 cells were resuspended in 1 ml PBS per cell line. Cells were then pelleted, resuspended in Fixation Buffer and fixed for 7 min at room temperature. Fixation Buffer consists of 2.8 ml H₂O, 790 μl 100% ethanol, 310 μl 40% glyoxal (Sigma 128465), 30 μl glacial acetic acid (Sigma A6283); after preparing Fixation Buffer, adjust the pH to 5.0 by adding NaOH and keep ice-cold until immediately before use. In line with a previous study³⁴, it was found that glyoxal fixation resulted in better preservation of intact nuclei than the more commonly used paraformaldehyde fixative.

After fixation, cells were then washed three times with 1 ml PBS and gently lysed by adding and resuspending in 10 ml Resuspension Buffer (see Bulk ATAC-seq above) with 0.1% Tween-20 and 0.1% Igepal CA630. Cells were then incubated on ice for 3 minutes and then pelleted at 500×g for 10 min at 4° C. to obtain nuclei. Nuclei were washed in 1 ml Tagmentation Buffer (see Bulk ATAC-seq above) with 5 μl RiboLock RNase Inhibitor (ThermoFisher E00381) and centrifuged at 500×g for 5 min at 4° C. Human and mouse nuclei were resuspended and mixed together in a final volume of 3.2 ml Tagmentation Buffer with 28 μl RiboLock RNase Inhibitor. Nuclei (30 μl, ˜20,000) were distributed into each well of a 96-well plate containing 20 μl of TnY assembled with MEDS A and 96 barcoded MEDS B. Tagmentation was performed for 30 minutes at 37° C. and then stopped by adding 2 μl EDTA 500 mM into each well. After incubating for 15 minutes at 37° C., EDTA was quenched prior to reverse transcription by adding 2 μl of 50 mM MgCl2 into each well.

For reverse transcription, 5 μl of the nuclei solution (˜2,000 nuclei) were transferred into a new 96-well plate containing barcoded reverse transcription primers. Reverse transcription primers contain the same barcode as the MEDS B oligos. Nuclei were transferred keeping plate orientation to match tagmentation and reverse transcription barcodes. The reverse transcription master mix (RTMM) consisted of 1 mL 5× RT buffer, 270 μl dNTPs, 1.6 mL water, 262 μl RevertAid reverse transcriptase, 27 μl RiboLock RNase Inhibitor (all components: Thermo Fisher, EP0442). 15 μl of RTMM was distributed into each well, mixed, and incubated for 30 min at 37° C.

Reverse transcription was stopped by adding 2 μl of Stop and Stain buffer (1 mL 500 mM EDTA, 2 μl 5 mg/ml DAPI) and incubated for 5 minutes on ice. Nuclei were pooled together and pelleted at 500×g for 5 min at 4° C. Supernatant was carefully removed taking care to not disturb the pellet. The nuclei were gently resuspended in 250 μl PBS and counted using a hemocytometer. PBS was added in order to obtain a final concentration of 10 nuclei/μl. 2 μl of the nuclei solution (˜20 nuclei) were transferred into a new 96-well plate with DNA extraction and digestion buffer in each well. Specifically, each well contained 24.5 μl of DNA Rapid Extract Buffer (1 mM CaCl₂, 3 mM MgCl₂, 1% Triton X-100, 10 mM Tris-HCl at pH 7.5) and 2 μl of Digestion Buffer (1 μl H₂O, 0.5 μl SDS 5.8%, 0.5 μl Proteinase K 20 mg/ml (Sigma P2308)). Nuclei were digested for 5 min at 65° C.; digestion was stopped by adding 3 μl PMSF (Sigma 93482) and incubating for 30 min at room temperature.

For the first PCR, ATAC-seq primers and sgRNA-PCR1 primers were added at a final concentration of 0.5 μM and 0.1 μM, respectively. Amplification for ATAC-seq/sgRNA-PCR1 was performed with PfuX7 in Phusion GC Buffer as follows: 72° C. 5 min, 98° C. 30 s, (98° C. 10 s, 63° C. 30 s, 72° C. 3 min)×14-18 cycles, 4° C. hold.

For the second PCR, 2 μl of PCR product were transferred into a new 96-well plate keeping plate orientation to match ATAC-seq and sgRNA barcodes. sgRNA-PCR2 primers were added to a final concentration of 0.5 μM. Amplification for sgRNA-PCR2 was performed with PfuX7 in Phusion GC Buffer as follows: 98° C. 30 s, (98° C. 10 s, 55° C. 10 s, 72° C. 20 s)×20 cycles, 72° C. 5 min, 4° C. hold.

ATAC-seq and sgRNA amplicons were purified. The ATAC-seq/sgRNA-PCR1 PCR plate was purified using four columns of the DNA Clean & Concentrator kit, eluted in 10 μl elution buffer and size-selected using 0.9× volume of Ampure XP Beads. The sgRNA-PCR2 PCR plate was purified using ten columns of the DNA Clean & Concentrator kit, eluted in 20 μl elution buffer. Eluted samples were run on E-gel 2% (Thermo Fisher G402002) and the expected band (˜250 bp) gel extracted, purified using 1 column of Zymoclean Gel DNA Recovery Kit (Zymo Research D4008) and eluted in 20 μl. Libraries were separately sequenced on the MiSeq Sequencer (Illumina) using the read lengths shown in FIG. 2B-FIG. 2E and custom primers as previously described^{35,36}.

CRISPR-sciATAC: Chromatin Modifier CRISPR Library

The CRISPR-sciATAC protocol for the chromatin modifier library in K562 cells was performed similarly to the human/mouse experiment described above. K562-Cas9 cells transduced with the pool of 63 chromatin modifiers sgRNAs and 3 non-targeting sgRNAs were grown for one week after selection. Twelve 96-well plates were prepared as described above and then pooled. The ATAC-seq amplicons were sequenced on a HiSeq 2500 (Illumina) and the sgRNA amplicons were sequenced on a MiSeq.

Essentiality Screen in K562 Cells

K562-Cas9 cells were transduced with the chromatin modifiers pooled CRISPR screen at MOI ˜0.1 and selected and maintained in 1 μg/ml puromycin and 5 sμg/ml blasticidin. Genomic DNA was extracted at three days (“Early Time Point”), one week and two weeks post-selection. The sgRNA cassette was PCR amplified as previously described²⁷. Libraries were sequenced on the MiSeq Sequencer. In addition to the CRISPR-sciATAC experiment, two independent transduction replicates were also analyzed.

sgRNA Alignment

Reads were trimmed with FASTX-Toolkit (hannonlab.cshl.edu/fastx_toolkit/), demultiplexed using grep (perfect match), and aligned to the 10 nontargeting human and 10 nontargeting mouse sgRNAs using bowtie³⁷ using the command bowtie -v 1 -m 1. Cells with at least 100 sgRNA reads were selected for further analyses. Cells with over 90% of sgRNA reads that mapped exclusively to human or mouse sgRNAs were considered species-specific cells. Cells where one sgRNA represented at least 90% of the total reads were kept for further analyses. The remaining cells were considered collisions and/or the result of multiple infections.

ATAC-Seq Alignment (Human/Mouse Mixture)

Reads were trimmed with FASTX-Toolkit, demultiplexed using grep (perfect match), aligned to the human hg19 and mouse mm10 reference genomes using bowtie2³⁸ using the command bowtie2 -D 15 -R 2 -L 22 -iS,1,1.15 -p 5 -t -X2000 -e 75—no-mixed—no-discordant and deduplicated using Picard (broadinstitute.github.io/picard). Cells with at least 500 unique ATAC-seq fragments were selected for further analyses. Cells with at least 90% of fragments mapping to the human or the mouse reference genomes were considered species-specific cells; the remaining cells were considered as collisions. Fragments overlapping ENCODE blacklist regions were filtered out (www.encodeproject.org/annotations/ENCSR636HFF/). ATAC-seq profiles of HEK293FT cells that passed ATAC-seq and sgRNA filters were compared to HEK293T DNaseI hypersensitivity peaks (www.encodeproject.org/experiments/ENCSR000EJR/) and to bulk HEK293FT ATAC-seq peaks.

ATAC-Seq Alignment (K562)

K562 sequence data was processed similarly to the human/mouse sequence data with a few differences outlined below. Guide alignments were demultiplexed based on cellular barcodes using the snATAC_mat.py script in a previously published sci-ATAC-seq pipeline (github.com/r3fang/snATAC)³⁹. For downstream analyses, each cell was required to have at least 100 aligned sgRNA reads with 99% of the reads assigned to one sgRNA sequence. All cells were aggregated into a “pseudo-bulk” dataset and peaks were called on this dataset with MACS2 (github.com/taoliu/MACS/)⁴⁰ using the following code macs2callpeak -g hs -p 0.05—nomodel—shift 150—keep-dup all.

Gene Essentiality Analysis

To identify essential genes, a p-value per sgRNA was calculated using the MAGeCK algorithm and p-values for the three sgRNAs targeting one gene were aggregated into a gene-level p-value using a Robust Rank Aggregation approach followed by a Bonferroni correction^{9,41}.

Differential Accessibility in TF Binding Sites Using ENCODE ChIP-Seq

To identify enrichment or depletion in accessibility of TF binding sites following chromatin modifier knock-out, 116 TF K562 ChIP-seq peak files were downloaded from ENCODE and considered the fraction of fragments in each single cell that overlap ChIP-seq peaks. To find significant deviations in accessibility per gene-KO and per TF, a two-tailed t-test was performed on the fractions, standardized over sgRNAs and over TFs into Z-scores, of all cells for one gene knock-out and all the non-targeting cells, for each TF. The p-values were adjusted for multiple hypothesis testing using a Benjamini-Hochberg false-discovery rate correction. For genes with multiple ENCODE ChIP-seq datasets, we denote with (1) ENCODE ChIP-seq profiles obtained using an antibody that directly recognizes the protein of interest; we denote with (2) ENCODE ChIP-seq profiles obtained using an antibody directed against an EGFP-tag.

Differential Accessibility in TF Binding Sites Using JASPAR Motifs

As an orthogonal method to ENCODE ChIP data, predicted TF binding sites from the JASPAR database was also utilized (386 motifs from JASPAR 2016, human CORE dataset)¹². Transcription factor motif enrichment and depletion scores were calculated using chromVAR20. Briefly, Z-scores quantifying deviations in the frequency of each motif in each of the single cells were calculated based on the frequency of the motif in the collection of peaks that exist in each cell, out of all 358,028 peaks called on the aggregated single cell alignment files (the “pseudo-bulk”). This frequency was compared to the frequency of the motif in peaks found in the entire aggregated single cell dataset¹³. We considered cells with a minimum of 2000 fragments per cell and a minimum of 10% of total fragments in peaks. To avoid biases from recovery of different numbers of cells for each sgRNA, we subsampled all sgRNA cell populations to 12 cells (the lowest number of cells for a single sgRNA in our K562 dataset), calculated the deviation Z-scores, and repeated this resampling process 1000 times to obtain deviation Z-scores for each sgRNA.

Nucleosome Positioning at AP-1 Sites

Coverage per base around AP-1 motifs using mononucleosomal fragments (defined as paired-end ATAC-seq fragments with a length between 180 and 247 nt³³) was calculated using BEDTools⁴². The nucleotide position of maximal coverage before and after the motif was used to compute the spacing between mono-nucleosomes. Smoothing was done using the R function smooth.spline with the smoothing parameter (spar) set to 0.5.

Differential Accessibility in Promoters and Enhancers

To identify significant changes in accessibility of enhancers and promoters, we calculated the coverage summed over transcription start sites and weak and strong enhancer midpoints. Weak and strong K562 enhancers were downloaded from UCSC (wgEncodeAwgSegmentation CombinedK562.bed from hgdownload.cse.ucsc.edu/goldenpath/hg19/encodeDCC/wgEncodeAwgSegmentation/). To avoid biases that may arise when comparing coverage between different gene-KOs with different numbers of single cells, we downsampled each cell population to 231 cells as the majority (18 out of 21 genes) have at least 231 cells. The remaining 3 genes with the lowest number of cells, CHD4, CHD8 and H3F3A, were downsampled to 124 cells and were compared to a non-targeting cell population of a similar size. Each population of cells was resampled 1000 times and the coverage at transcription start sites, weak enhancers (midpoint), and strong enhancers (midpoint) was calculated. Empirical p-values were calculated for each gene by averaging these values and comparing them to a null distribution derived from non-targeting cells over 1000 resampling iterations.

Accessibility Analysis at Genomic Regions with Specific Chromatin and DNA Modifications

To assess changes in accessibility, we downloaded from ENCODE ChIP-seq files covering posttranslational histone modifications and DNA methylation. For each ChIP-seq track, we considered the fraction of fragments in each single cell that overlap ChIP-seq peaks. We averaged the fractions obtained for each ChIP-seq file over cells that received the same sgRNA and standardized the averaged fractions over the sgRNAs into Z-scores.

GO Analysis of Differential EZH2 Chromatin Accessibility Sites

In order to identify and annotate genomic regions that are differentially accessible in cells with EZH2-targeting sgRNAs, we aggregated equal numbers of single cells (n=170 cells per sgRNA) for each of the three EZH2 and non-targeting sgRNAs. We next binned the genome into 150 nt regions and identified all bins covered by all three EZH2 sgRNAs and not covered by any of the three non-targeting sgRNAs. These bins were then mapped to the transcription start site of the closest genes. We used this (unranked) gene list (n=3,740) as input for Gene Ontology enrichment analysis, with all human genes as a background set⁴³.

Differential Accessibility at HOX Loci

EZH2-targeted and non-targeting single cells were downsampled to 100 cells, aggregated and fragments overlapping the HOXA-D loci were counted. Empirical p-values were calculated over 1000 bootstrap iterations.

pLI Scores

We obtained probability for loss-of-function intolerance (pLI) scores from the Genome Aggregation Database (gnomAD)^{44,45}, which contains 15,708 whole genomes and 125,748 whole exomes. pLI scores are bounded from 0 to 1, where scores closer to 1 are strongly indicative of intolerance to protein-truncating loss-of-function variants. We used a threshold of pLI>0.9 to identify intolerant genes, as previously suggested^{44,45}.

eQTL Enrichment

To test if targeting chromatin modifiers resulted in changes in accessibility at SNPs associated with regulatory function through expression quantitative trait locus (eQTL) association testing, we utilized cis-eQTLs (SNP-gene combinations within 1 Mbp) from the eQTLGen consortium. The consortium performed association testing for 19,960 genes expressed in blood in 31,684 samples⁴⁶. We considered the fraction of fragments in each single cell that overlap cis-eQTLs and compared these fractions for each population of single cells that received sgRNAs targeting a gene to the fractions in non-targeting cells using a Wilcoxon signed-rank test followed by a Benjamini-Hochberg multiple hypothesis correction.

Standard Statistical Analysis

Data between two groups were analyzed using a two-tailed unpaired t-test or a non-parametric Wilcoxon signed-rank test. The p values and statistical significance were estimated for all analyses. In all the box plots, the central rectangle in the plot covers the first to the third quartile (the interquartile range, or IQR). The bold line is the median. The whiskers are defined as: Upper whisker=min(max(x), Q_3+1.5×IQR) and lower whisker=max(min(x), Q_1−1.5×IQR). All statistical analyses were performed in R/RStudio.

### Example 2—Scalable Pooled Crispr Screens with Single Cell Chromatin Accessibility Profiling

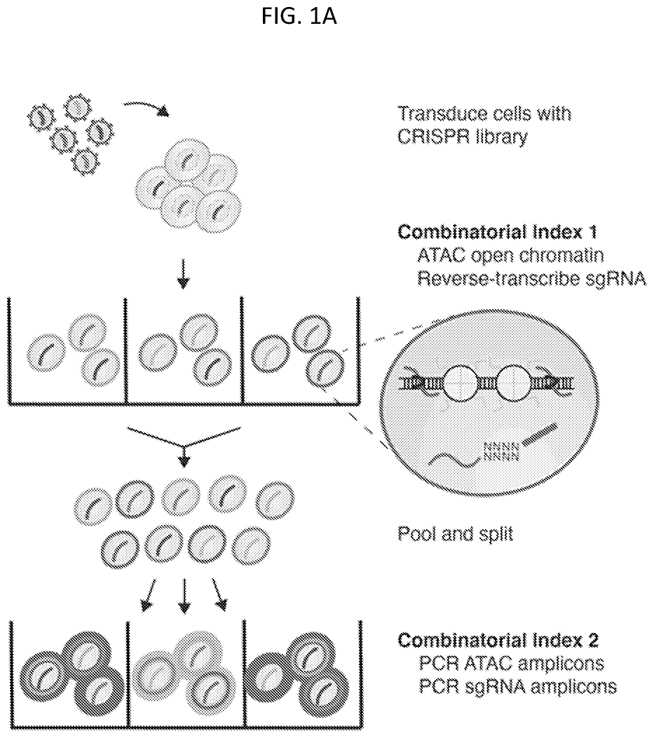

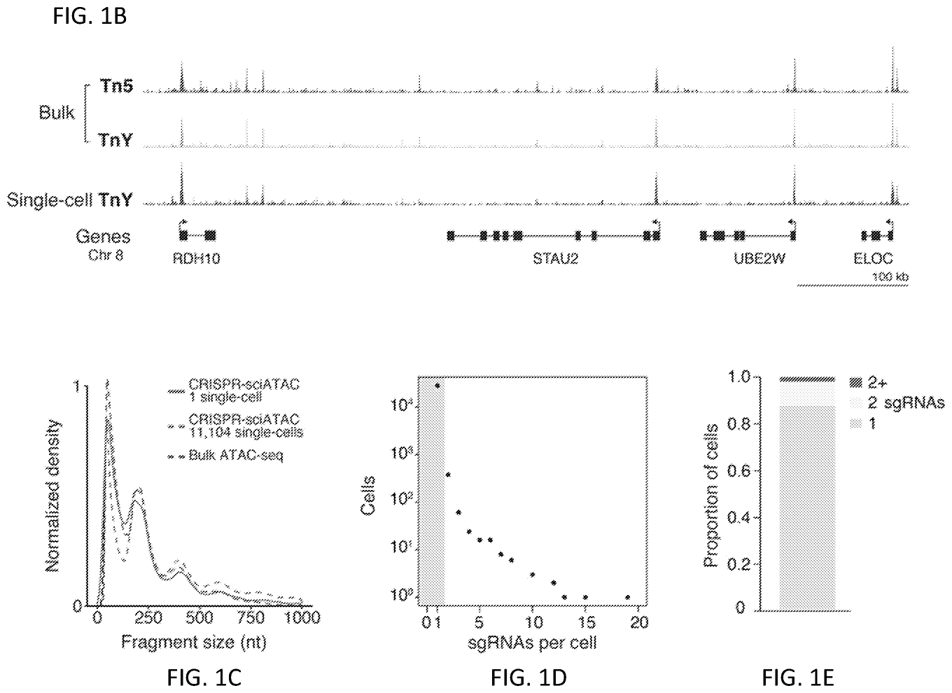

To study how genetic perturbations affect chromatin states and cellular phenotypes, a novel platform was developed for scalable pooled CRISPR screens with single-cell ATAC-seq profiles: CRISPR-sciATAC. In CRISPR-sciATAC, we simultaneously capture Cas9 single-guide RNAs (sgRNAs) and perform single-cell combinatorial indexing ATAC-seq′ (FIG. 1A and FIG. 2A). Following cell fixation and lysis, nuclei are recovered and the open chromatin regions of the genomic DNA undergo barcoded tagmentation in a 96-well plate using a unique, easy-to purify transposase purified from Vibrio parahemolyticus (FIG. 1B, FIG. 3A-FIG. 3G). Next, the sgRNA is barcoded with the same barcode as the ATAC fragments, using in situ reverse transcription. The nuclei are pooled together and split again to a new 96-well plate and both the ATAC fragments and the sgRNA are tagged again with a well-specific barcode in two consecutive PCR steps. At the end of this process, every single cell contains a unique combination of barcodes that tag both the sgRNA and the ATAC fragments with the same barcode combination (“cell barcode”) (FIG. 1A, FIG. 2A-FIG. 2E). Since CRISPR-sciATAC is plate-based and uses a unique, easy-to-purify transposase (FIG. 3A-FIG. 3H), ATAC-seq libraries from thousands of single cells can be prepared in a single day.

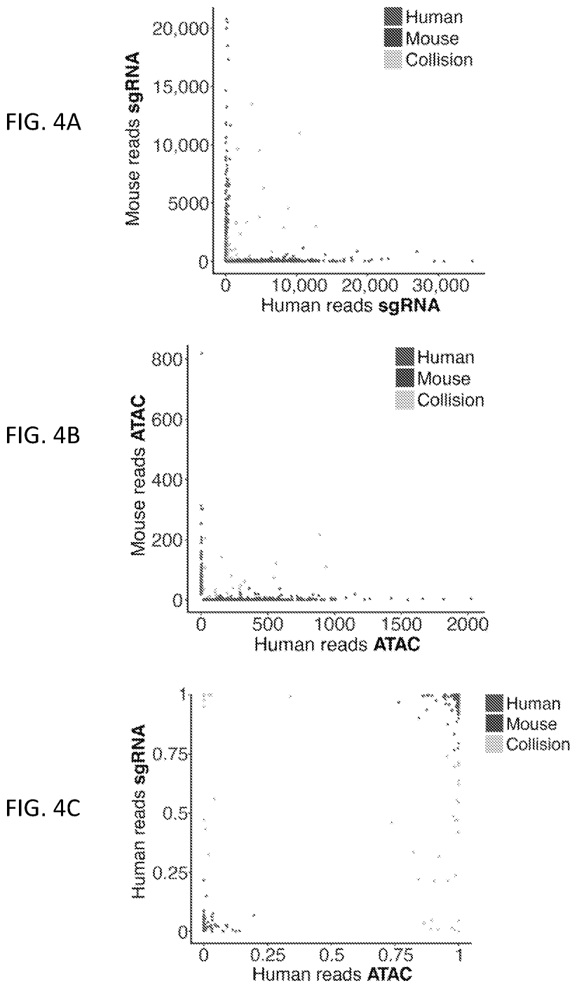

To test the ability of CRISPR-sciATAC to adequately barcode and capture single-cells, we performed CRISPR-sciATAC on a mix of human (HEK293) and mouse (NIH3T3) cells. Human and mouse cells were each transduced with a small library of 10 distinct non-targeting sgRNAs with no overlapping sgRNAs between the two pools. We found that 93% of cell barcodes had sgRNA-containing reads that could uniquely be assigned to either human or mouse sgRNAs (FIG. 4A) and 96% of cell barcodes had ATAC-seq reads mapping to either the human or mouse genome, indicating that the majority of cell barcodes were correctly assigned to single cells (FIG. 4B). As an additional verification of single-cell separation, we also measured the species concordance between the ATAC-seq and sgRNA reads. We found that for 92% of the captured cell barcodes both ATAC-seq and sgRNA reads aligned either to human or mouse reference genomic and sgRNA sequences, respectively. In 4.4% of cells, the ATAC-seq and/or sgRNA reads could not be exclusively assigned to a species. ATAC-seq and sgRNA reads were assigned to different species (ATAC-seq and sgRNA species collision) in 3.6% of cells (FIG. 4C). The low rates of these two failure modes suggest that CRISPR-sciATAC can simultaneously identify accessible chromatin and CRISPR sgRNAs from single cells.

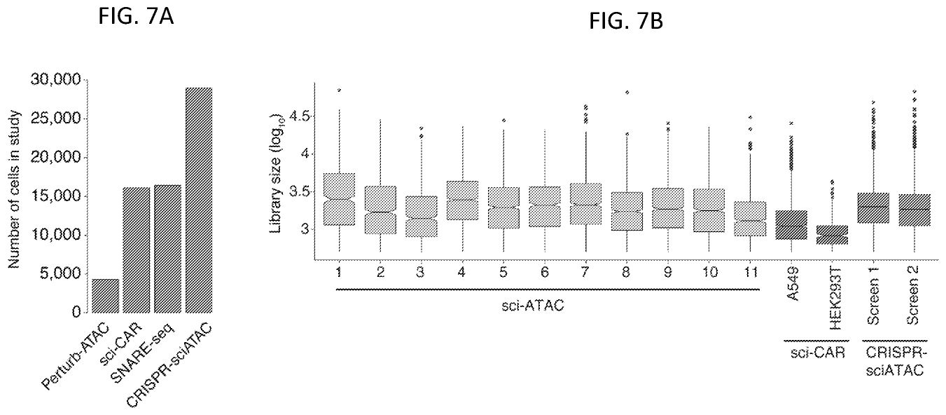

To test the ability of CRISPR-sciATAC to capture biologically meaningful changes in chromatin accessibility, we targeted 21 chromatin modifiers that are highly mutated in cancer (FIG. 5A and FIG. 5B). Using the Catalog of Somatic Mutations in Cancer (COSMIC) database⁸, we selected 21 chromatin-related genes that carry the highest mutational load (mutations per coding base) across all cancers, including 9 chromatin remodelers (ARID1A, ATRX, CHD4, CHD5, CHD8, MBD1, PBRM1, SMARCA4, and SMARCB1), 2 DNA methyltransferases (DNMT3A and TET2), 3 histone methyltransferases (EZH2, PRDM9, and SETD2), 1 histone demethylase (KDM6A), 1 histone deacetylase (HDAC9), 3 histone subunits (H3F3A, H3F3B, and HIST1H3B), and 2 readers (ING1 and PHF6) (FIG. 5B). We designed 3 sgRNAs to target the coding exons of each gene and also included 3 non-targeting sgRNAs in our library (FIG. 19A and FIG. 19B). After filtering for cells with 500 unique ATAC-seq fragments and 100 sgRNA reads (FIG. 5C-FIG. 5F), we obtained 11,104 cells with a median of 1,977 unique ATAC-seq fragments mapping to the human genome, comparable to other sciATAC studies (FIG. 7A and FIG. 7B). Single cells retained a nucleosome position dependent fragment length distribution similar to cells tagmented in bulk (FIG. 1C). The majority of cell barcodes (83%) had one sgRNA (FIG. 1D and FIG. 1E).

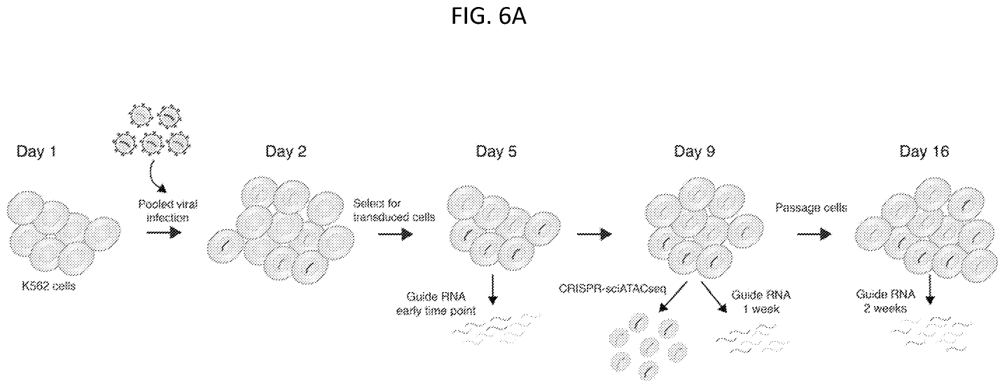

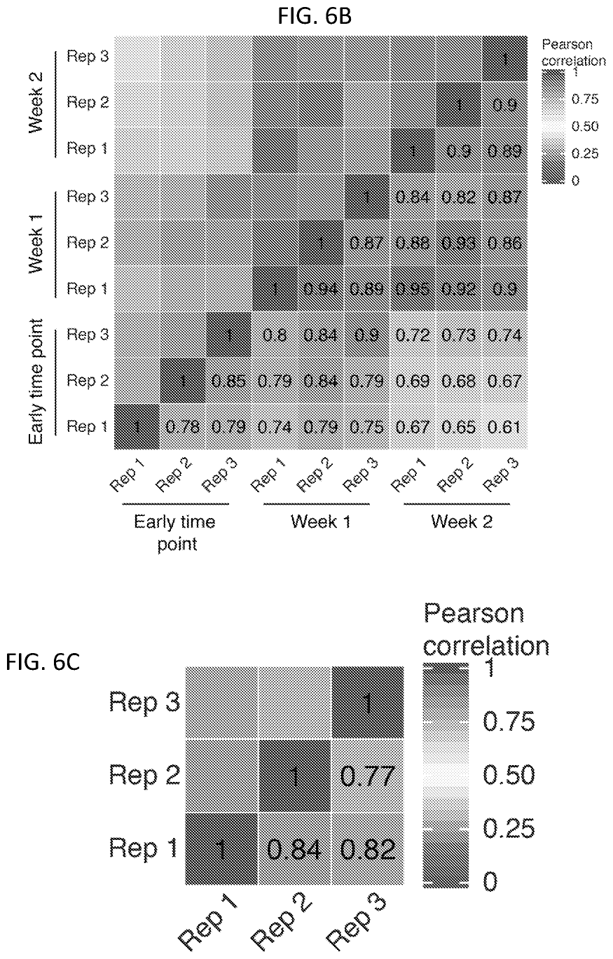

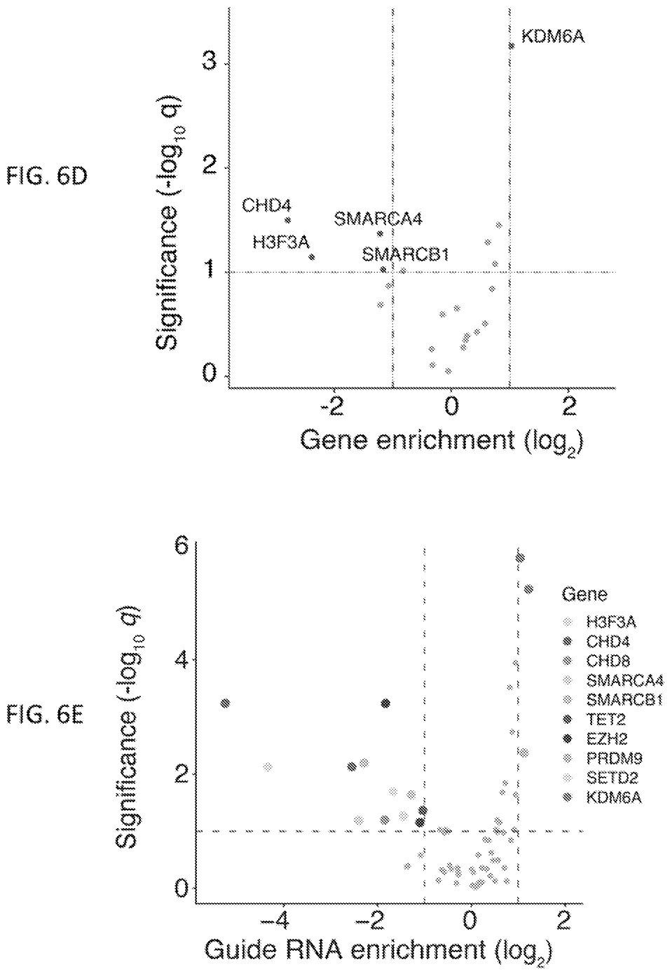

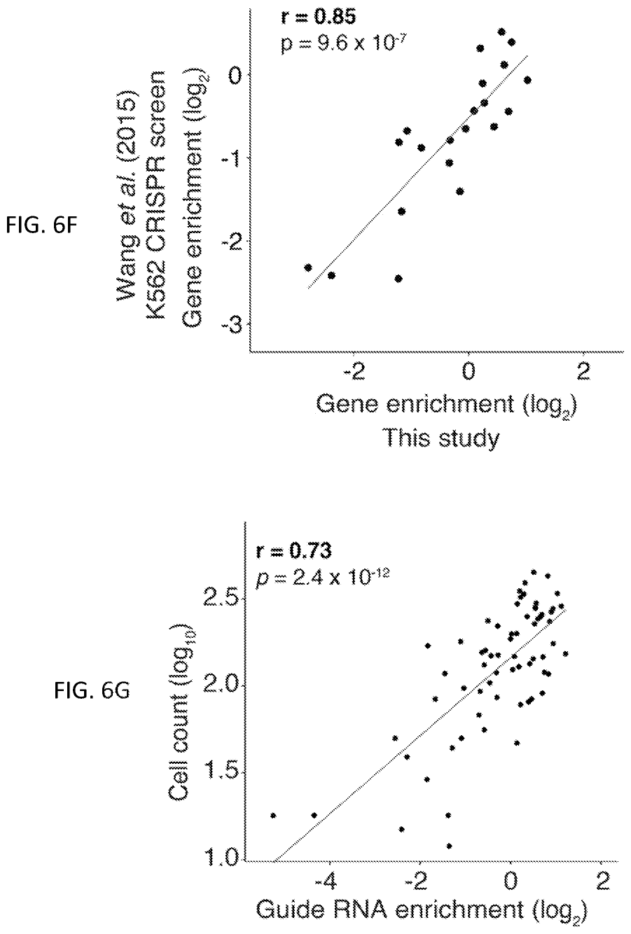

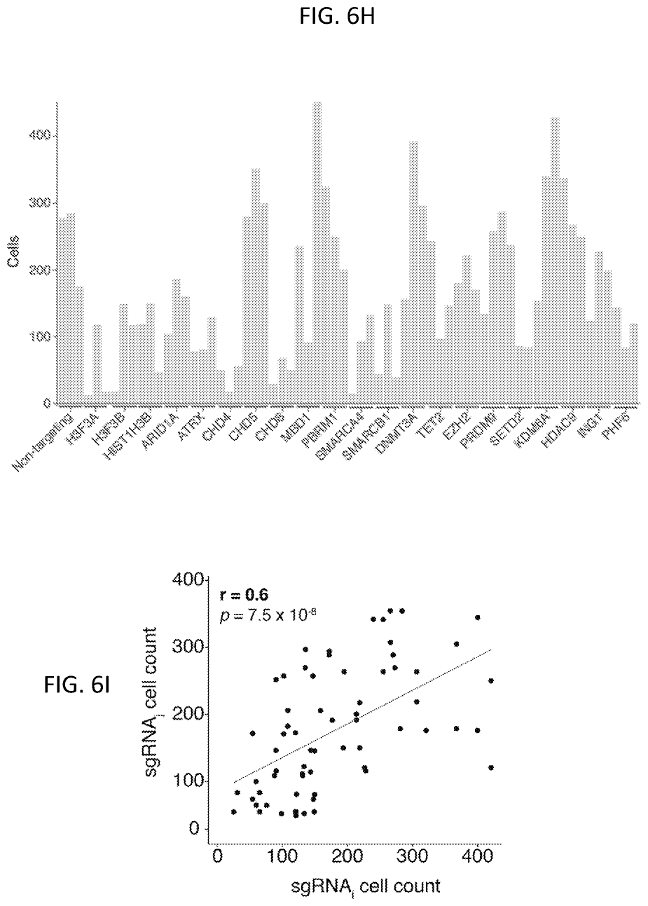

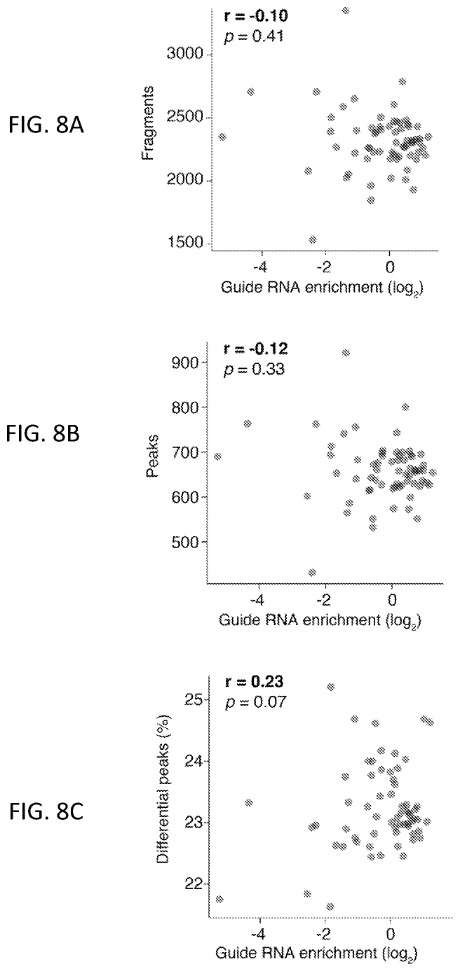

We recovered all of the 66 sgRNAs with a median of 148 single cells per sgRNA and 468 single cells per gene (FIG. 6H, FIG. 19A and FIG. 19B). Upon closer examination, we noticed that not all gene targets resulted in the same number of single-cells captured, suggesting that some of our targets might be essential genes whose targeting leads to drop-out of those cells. To distinguish sgRNA depletion of essential genes from inability to capture sgRNAs using CRISPR-sciATAC, we amplified sgRNAs from the population of cells at an early time point and at 1 and 2 weeks post-selection (FIG. 6A). We found high correlations between all samples across 3 independent transduction replicates (FIG. 6B and FIG. 6C). For several genes, multiple, distinct sgRNAs targeting the same gene were consistently depleted or enriched: H3F3A, CHD4, SMARCA4, and SMARCB1 were consistently depleted, while targeting KDM6A resulted in accelerated cell growth (FIG. 6E). Using robust rank aggregation to measure consistent enrichment across multiple sgRNAs9, we computed gene-level enrichment scores (FIG. 6D, FIG. 19A and FIG. 19B), which were highly correlated with a previous genome-wide CRISPR screen in K562 cells 10 (r=0.85, FIG. 6F). Reassuringly, enrichment of individual sgRNAs was positively correlated with cell numbers estimated from CRISPR-sciATAC cell barcodes (r=0.73, FIG. 6G). Different sgRNAs targeting the same gene tend to result in similar numbers of single cells, highlighting consistent proliferation phenotypes between different genetic perturbations targeting the same gene (FIG. 6I). We did not observe changes in the number of ATAC fragments per cell between the different perturbed genes (and gene enrichment was not correlated with the number of ATAC fragments, peaks, or differential peaks obtained from sgRNAs targeting the same gene (FIG. 8A-FIG. 8C).

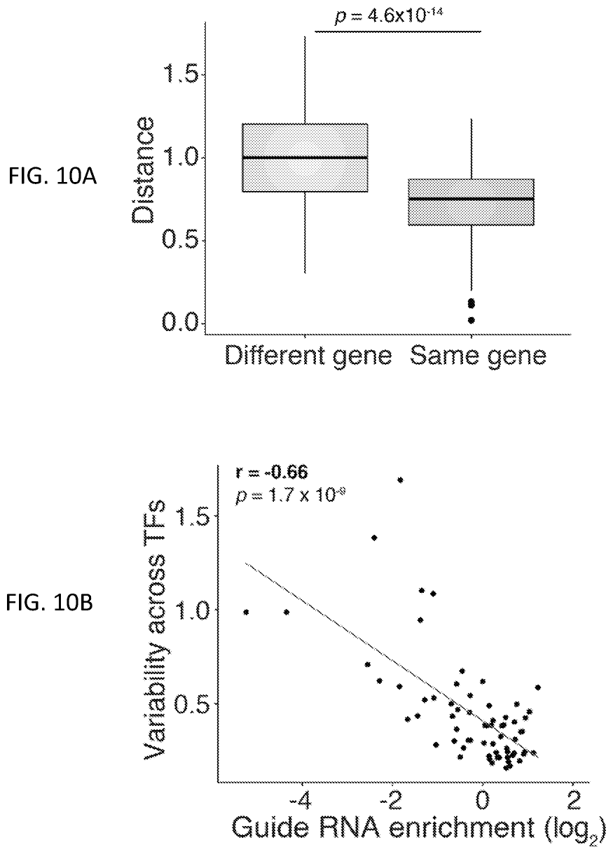

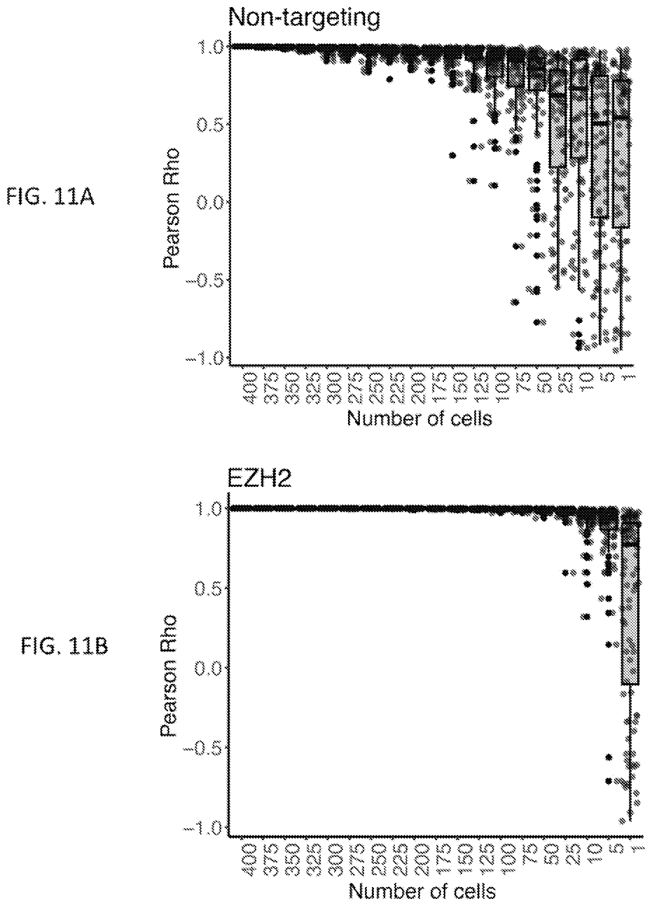

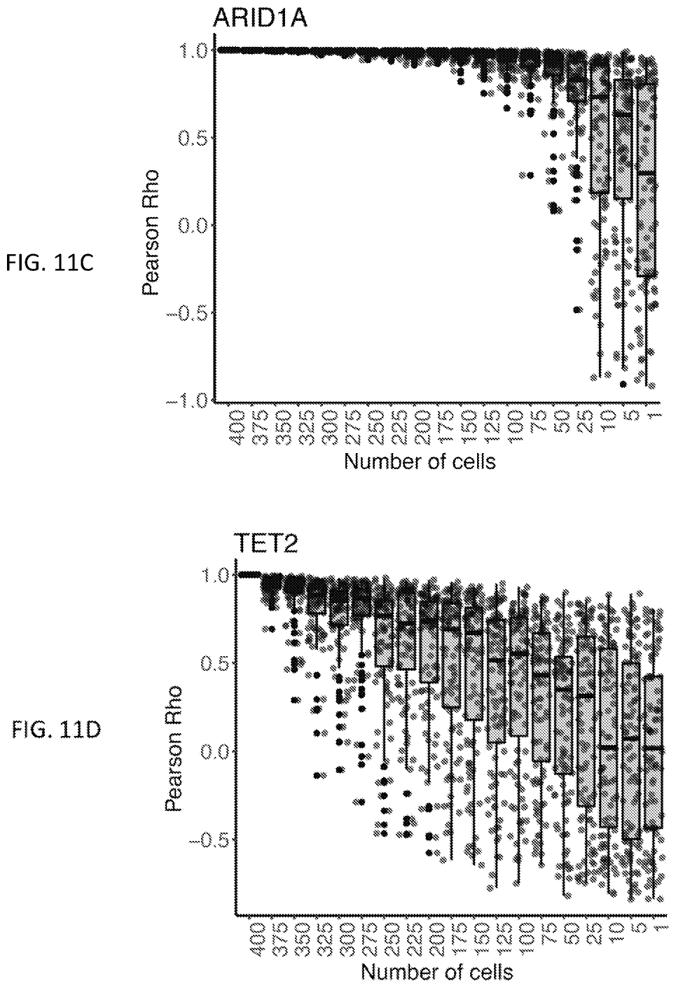

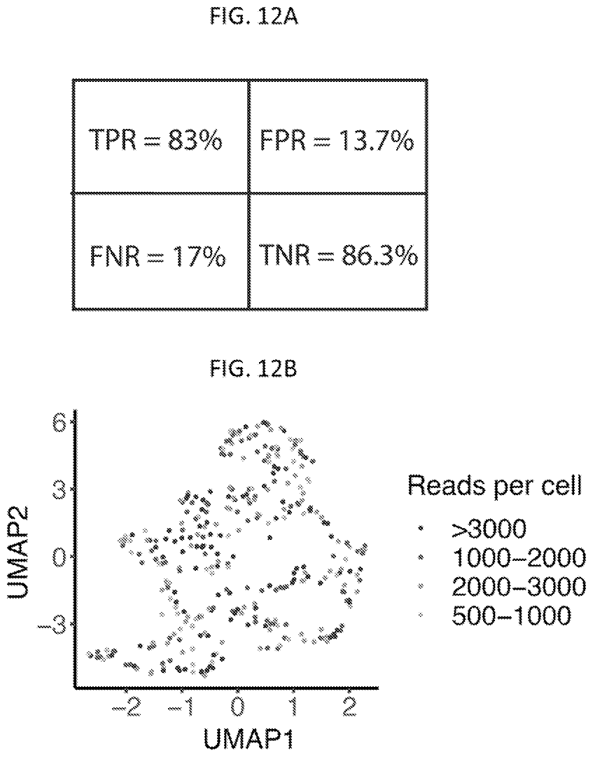

We next examined how loss-of-function of these genes affects accessibility within known chromatin marks (histone post-translation modifications) using ENCODE K562 data (FIG. 9A). We found similar accessibility changes between different sgRNAs targeting the same genes, further highlighting the consistency between distinct genetic perturbations targeting the same gene (FIG. 9B). The changes in accessibility in single cells at transcription factor binding site (TFBS) peaks are similarly consistent between sgRNAs targeting the same gene (FIG. 10A). Targeting the Polycomb repressive complex (PRC2) subunit EZH2 resulted in a strong increase in chromatin accessibility at H3K27me3 regions, a marker of heterochromatin (FIG. 9A). EZH2 catalyzes nucleosome compaction via H3K27 trimethylation²¹ and thus loss of EZH2 increases accessibility in these regions. A down-sampling analysis of single cells reveals that in the case of EZH2, as little as 5 cells correlate well (Pearson's rho>=0.75) to an aggregated, “pseudo-bulk” cell population (FIG. 9C, FIG. 11B). For non-targeting cells, 75 cells are able to represent the pseudo-bulk (FIG. 11A, median over all targeted genes=75 cells).

A uniform manifold projection (UMAP) projection of the histone accessibility profiles reveals a visible separation between single cells transduced with EZH2-targeting sgRNAs and single cells transduced with non-targeting sgRNAs (FIG. 9D). We verified this separation is not due to differences in library complexity in cells with EZH2-targeting sgRNAs (FIG. 12C). Applying a logistic regression classifier to differential TFBS accessibility, we found that increased accessibility in Polycomb repressive complex 1 (PRC1) components CBX2 and CBX8 has the highest predictive power in differentiating EZH2-targeted cells from cells (FIG. 9D). Reassuringly, we also saw an increase in accessibility at EZH2 sites, which is expected given EZH2's role in repression through heterochromatin formation (CITE). We also found that decreased accessibility of POL2B and SIRT6 in cells with EZH2-targeting sgRNAs (FIG. 9D).

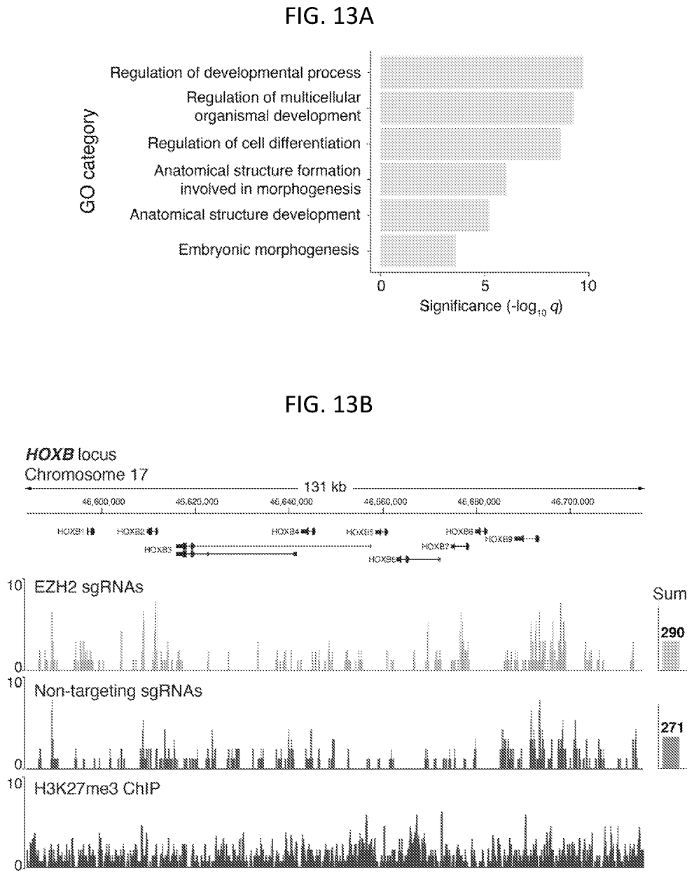

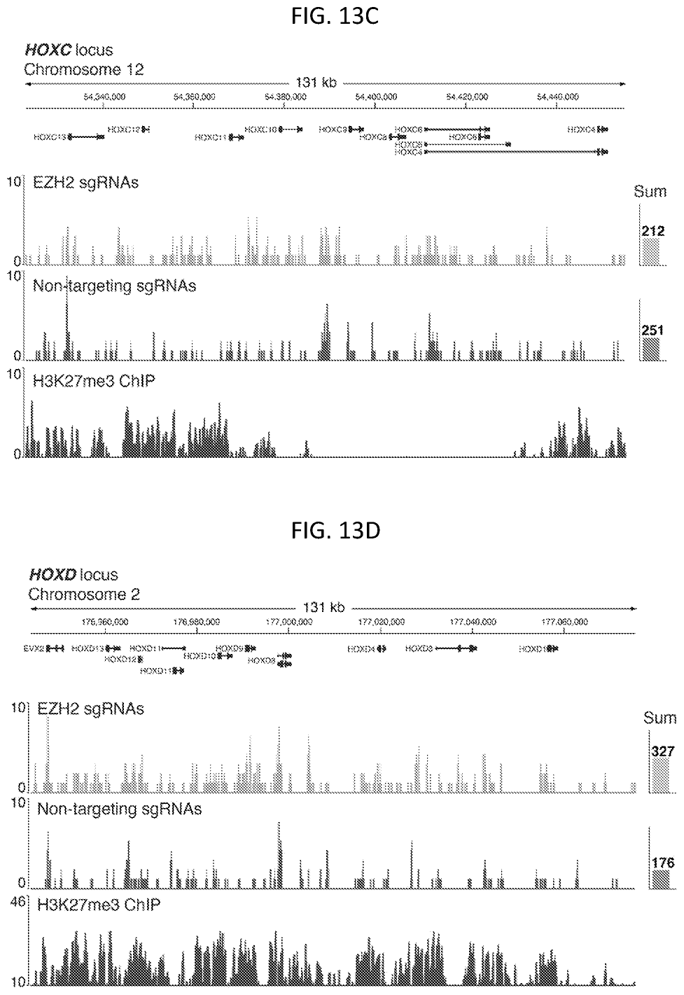

Using Gene Ontology (GO) analysis of differentially accessible regions in EZH2-targeted cells, we found an enrichment in genes involved in embryonic development and cell differentiation (FIG. 13A). Indeed, EZH2 is known to play important roles in embryonic development and cell- and tissue-specific differentiation²¹ and we found large changes in chromatin accessibility at several of the homeobox (HOX) genes (FIG. 9E and FIG. 9F and FIG. 13B-FIG. 13D). In K562 cells, the HOXA and HOXD gene clusters contain the highest amount of the H3K27me3 repressive heterochromatin mark (FIG. 9E). In the HOXA gene cluster, we found that there was a nearly 3-fold increase in accessibility (FIG. 9F). A similar increase in accessibility was also seen at the HOXD gene cluster (FIG. 9E, FIG. 13D).

To understand the functional consequences of these changes, we measured the expression of EZH2 and several HOX genes (HOXA3, HOXA5, HOXA11, HOXA13, and HOXD9) (FIG. 9G). After EZH2 loss, we found that these genes become highly expressed. Since we had 3 sgRNAs targeting EZH2, we also noticed that the sgRNA that was least efficient for EZH2 knock-out and also resulted in smaller increases in expression for all 5 of the HOX genes that we assayed. Taken together, these results suggest that loss-of-function mutations in EZH2 lead to aberrant expression of HOX genes.

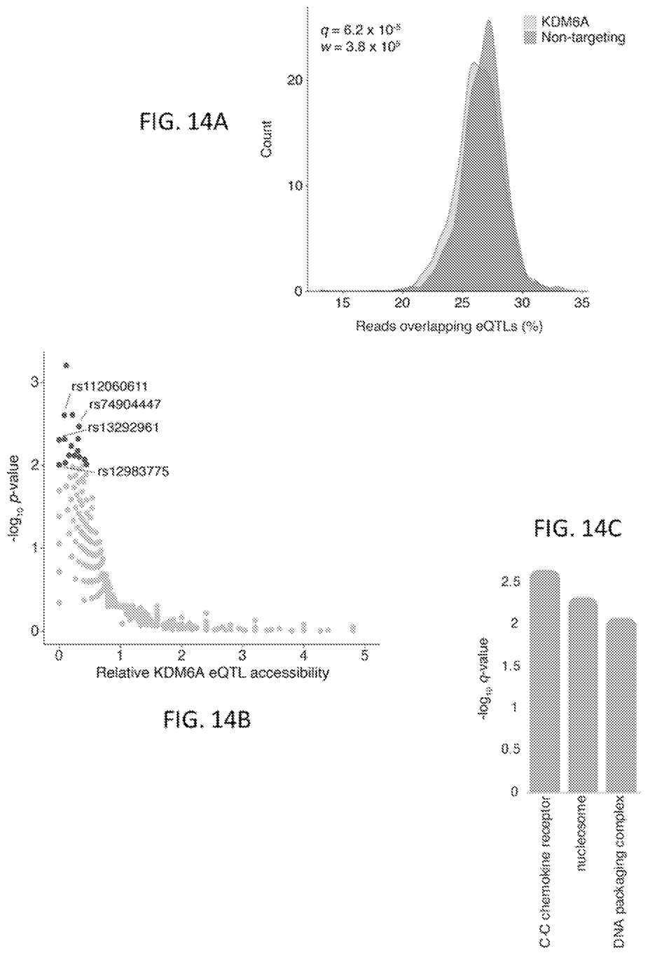

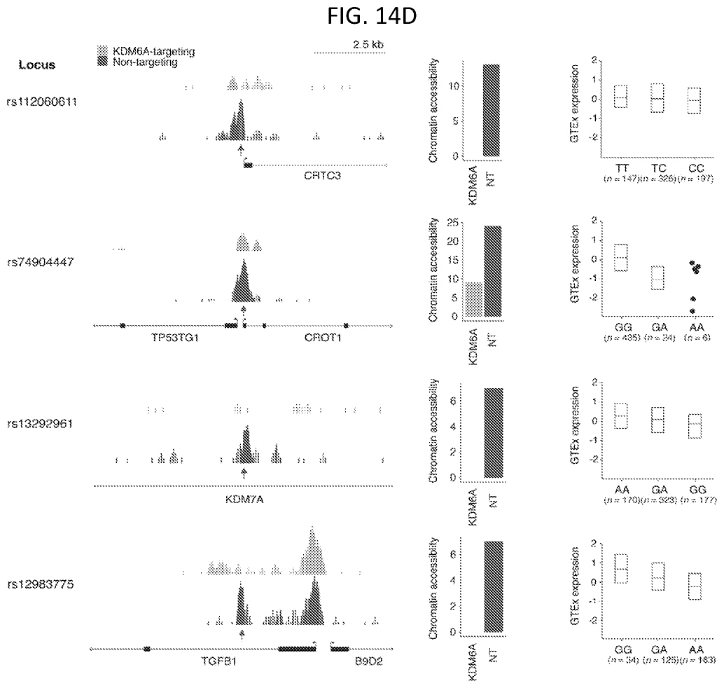

We assessed the relationship between chromatin accessibility changes due to loss-of-function mutations and human genetic variation. To determine if chromatin accessibility is modified at single nucleotide polymorphisms (SNPs) that regulate gene expression, we measured overlap with cis-regulatory expression quantitative trait loci (cis-eQTLs). For two of our targets—KDM6A and ARID1A—we found a reduction in accessibility at tissue-matched (blood) cis-eQTLs in cells after perturbation of these genes. The most pronounced reduction of accessibility is in the gene KDM6A (FIG. 14A) with the largest changes in genes involved in DNA condensation and chemokine receptor activity (FIG. 14B and FIG. 14C).

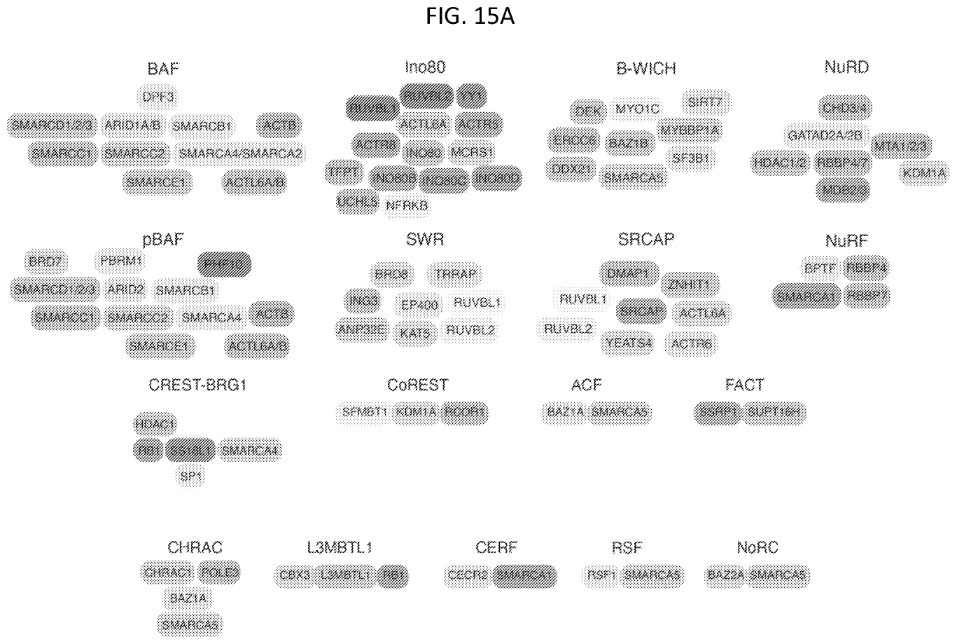

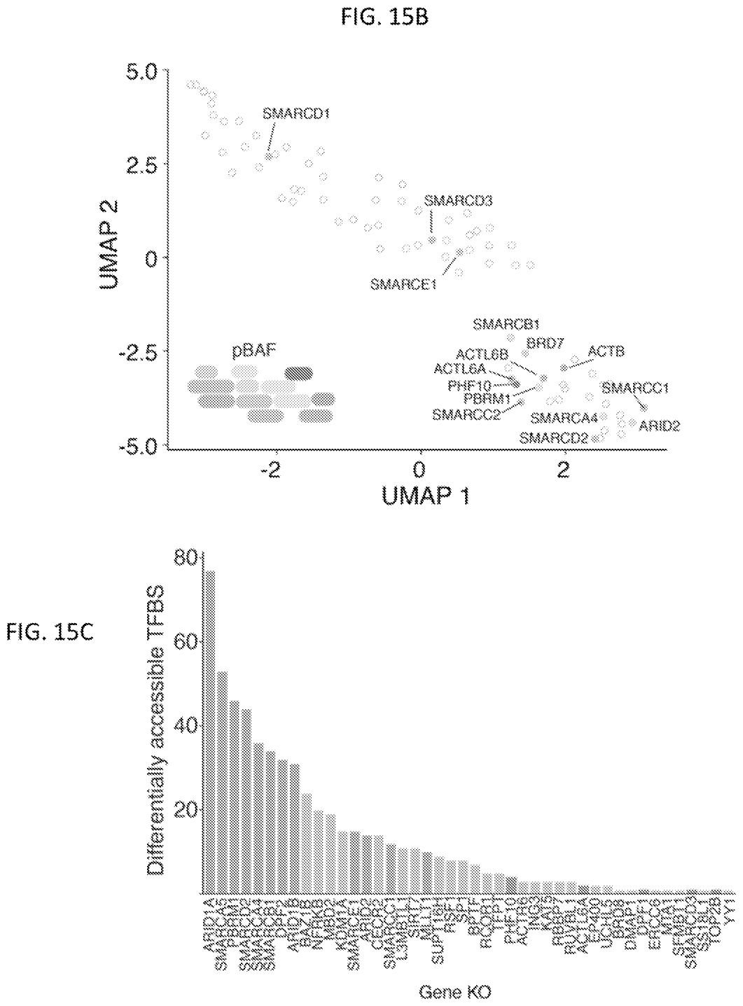

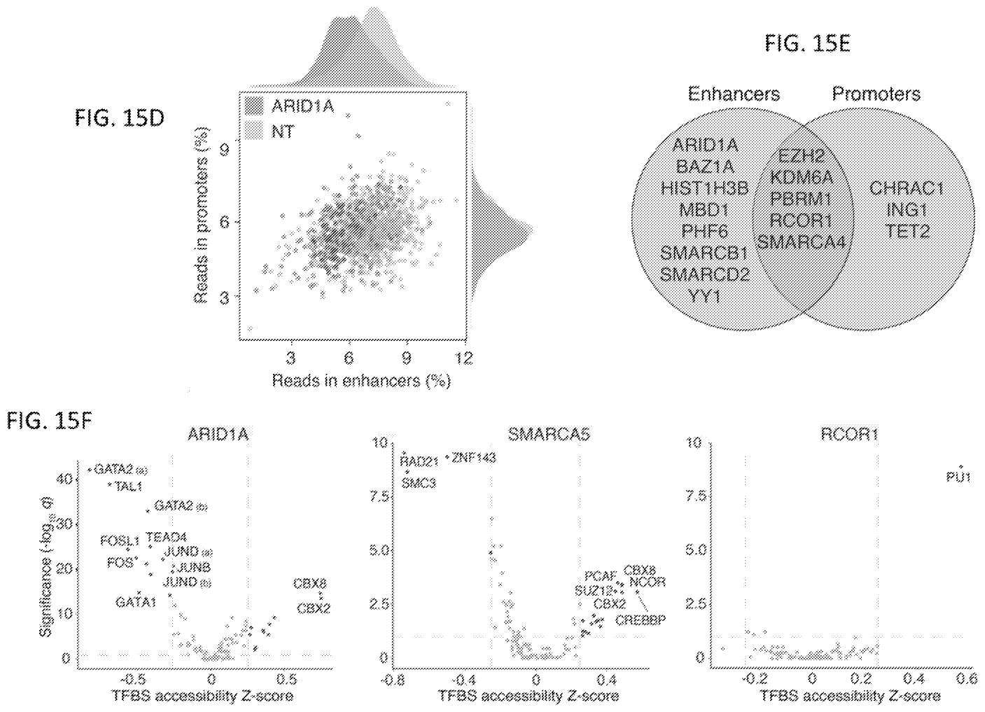

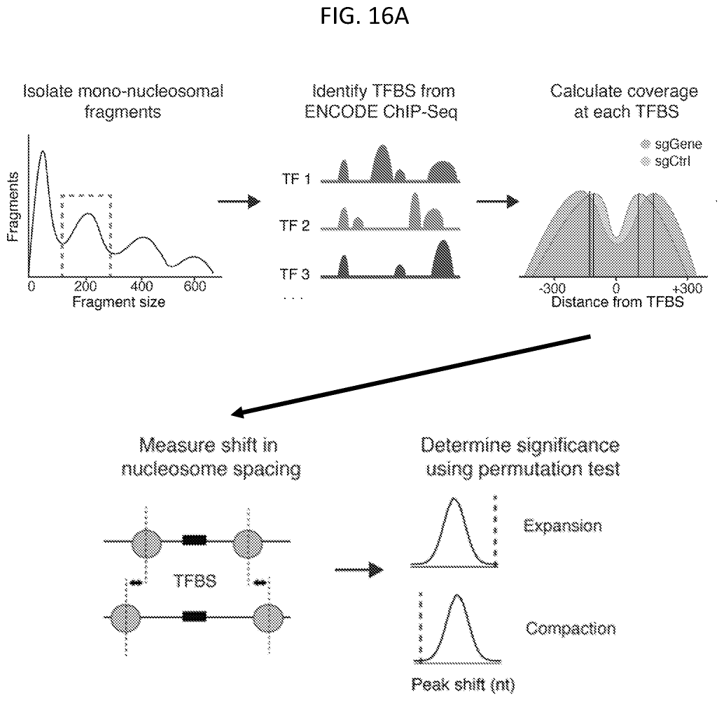

To demonstrate the scalability of CRISPR-sciATAC, we designed a CRISPR library to target all chromatin remodeling complexes in the human genome, as defined by the EpiFactors database [PMID: 26153137] (FIG. 15A). In total, we targeted 17 chromatin remodeling complexes and each complex consistent of between 2 and 14 subunits. We targeted the coding exons of each subunit with 3 sgRNAs and also included sgRNAs designed not to target anywhere in the human genome in the library. Over the 17 chromatin remodeling complexes, we captured paired CRISPR perturbation and single-cell ATAC-seq data from 16,676 cells.

Chromatin accessibility at specific DNA sequences allows TFs to bind while the presence of nucleosomes or other proteins can create steric hindrance that prevents physical interaction¹¹. In order to identify differential TF binding following perturbation of chromatin remodeling complexes, we analyzed changes in accessibility in single cells at TFBS peaks in ENCODE K562 chromatin immunoprecipitation sequencing data. We analyzed changes in accessibility at TFBSs resulting from targeting different chromatin remodeling complexes (FIG. 15A). Hierarchical clustering of these profiles revealed two major group: One group consisting of most increases in accessibility, such as the ATP-utilizing chromatin assembly and remodeling factor protein (ACF) and the nucleolar remodeling (NoRC) complexes, and another group consisting of decreases in accessibility, such as CECR2-containing remodeling factor (CERF) and corepressor for element-1-silencing transcription factor (CoREST) complex.

A two-dimensional UMAP projection of the TFBS accessibility profiles reveals a cluster containing a distinct signature of pBAF components but not BAF (FIG. 15B). Knocking-out SWI/SNF subunits changes accessibility at many TFBS, with the largest number of changes caused by ARID1A loss (FIG. 15C). Previously, ARID1A loss has been shown to impair enhancer-mediated gene regulation [PMID: 27941798], and indeed we find that loss of ARID1A dramatically reduced accessibility at strong and weak enhancers, but not at promoters (FIG. 15D).

Changes in chromatin accessibility at enhancers helps orchestrate the interactions between promoters and distal regulatory regions, which in turn is a key regulator of gene expression¹⁸. Combining data from both CRISPR-sciATAC experiments, we found that perturbation of chromatin modifiers has a stronger impact on enhancers than at promoters (FIG. 15E), supporting a gene regulatory model with more dynamic chromatin accessibility at distal regulatory elements compared to promoters¹⁹. Profiling chromatin accessibility at promoters and enhancers revealed several genes whose perturbation significantly altered accessibility at one or more of these regulatory regions (FIG. 15E). Loss of SWI/SNF-ATPase subunit ARID1A and loss of ISWI-ATPase subunit SMARCA5 show a wide effect of disruption in accessibility in binding sites of tens of TFs (FIG. 15C). Specifically, we noted that loss of ARID1A triggered a reduction in accessibility at JUN and FOS binding sites, which are subunits of the AP-1 transcription factor (FIG. 15F). AP-1 has been shown to cooperate with the SWI/SNF complex to regulate enhancer activity¹⁶. Loss of SMARCA5 triggered a reduction in accessibility in binding sites of cohesin subunits RAD21 and SMC3 along with cohesin cofactor ZNF143 [PMID: 30552588]. SMARCA5 has been hypothesized to be important in the loading of cohesion onto chromosomes [PMID: 12198550]. In contrast to these genes affecting a wide range of TFBSs, others have a specific effect on a limited number of TFBSs. RCOR1 has been suggested to promotes erythroid differentiation by repressing myeloid genes such as PU.1 [PMID: 24652990]. In our data, we observed an increase in accessibility in PU.1 binding sites in RCOR1-targeted cell populations (FIG. 15F).

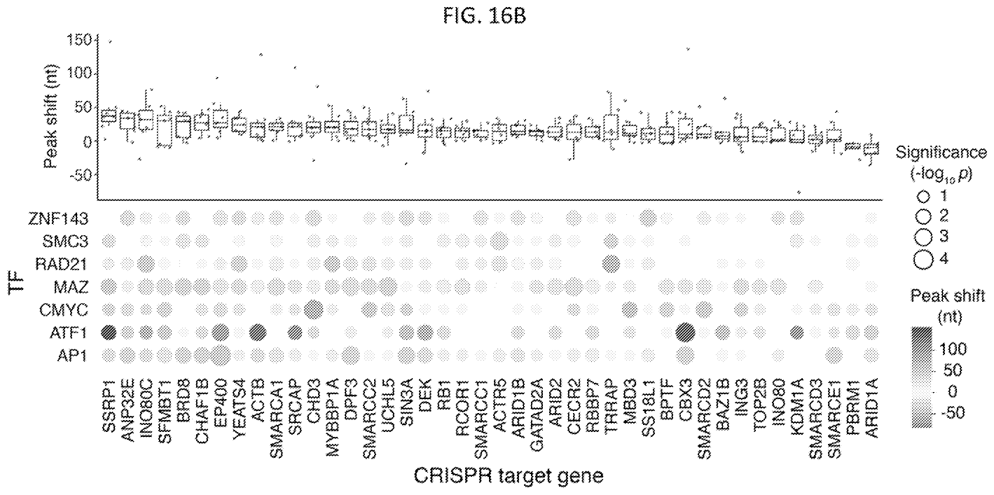

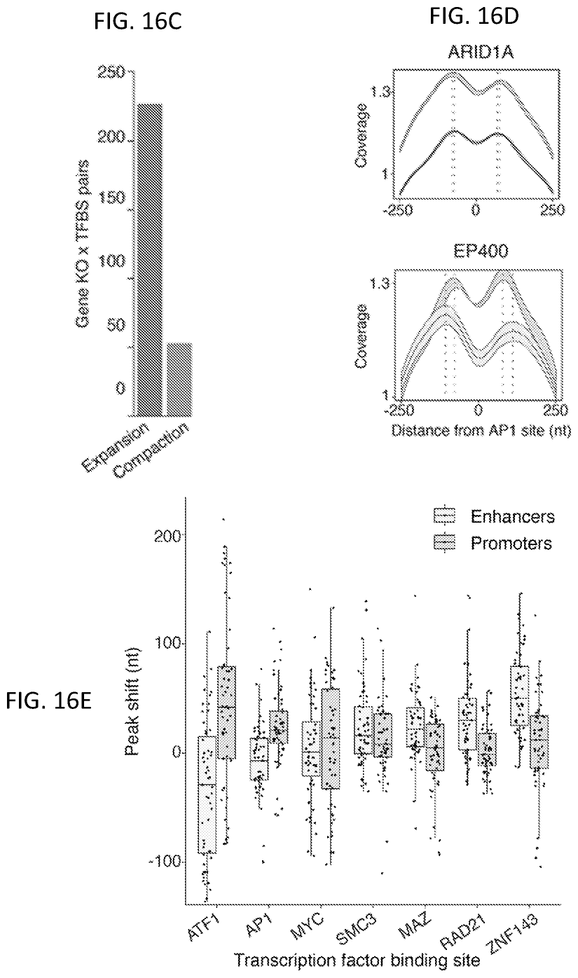

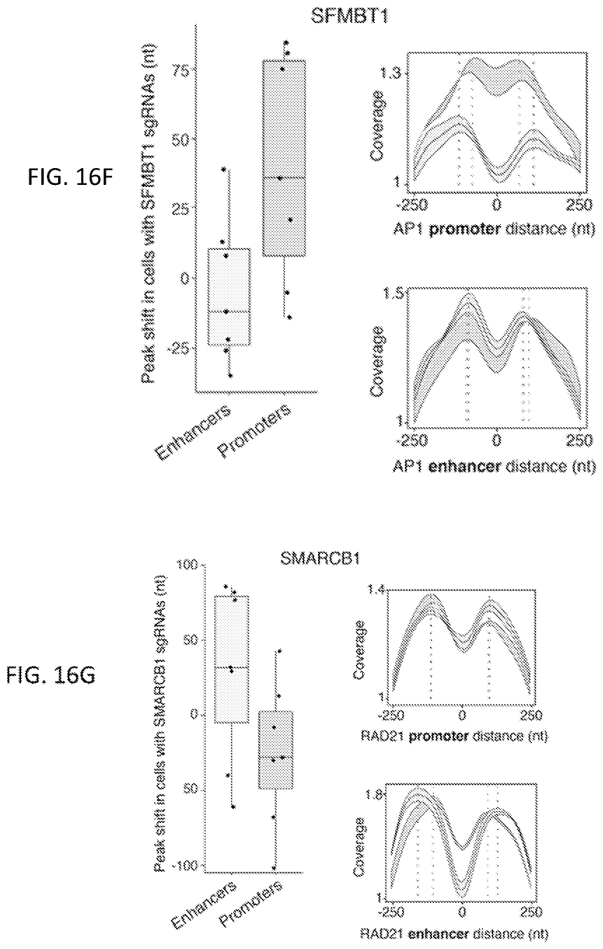

Chromatin remodeling complexes can regulate gene expression by sliding nucleosomes around regulatory genomic sequences such as TFBSs. Some TFs have a highly structured and symmetric positioning of nucleosomes around their binding sites [PMID: 22955985], and the distance between these nucleosomes allows or prevents access of TFs to their binding sites. We studied the effect of knocking out chromatin remodeling genes on the accessibility of TFBSs via the identification of changes in nucleosome positions around TFBSs in KO cell populations (FIG. 16A). We found that chromatin remodeling genes such as SSRP1, ANP32E, INO80C and EP400 caused expansion of nucleosomes around the TFBSs studied (FIG. 16B). Disruption of chromatin remodeling genes generally results in expansion of nucleosomes around TFBSs (FIG. 16C), with the exception of BAF/pBAF subunits ARID1A and PBRM1 whose knock-out causes the compaction of nucleosomes around the TFBSs studied (FIG. 16B).

At specific TFBS, loss of different chromatin remodelers can have opposing effects: For example, ARID1A loss results in a 20 nt nucleosome compaction at AP-1 binding sites (p=0.034) which has also been demonstrated in a recent study suggesting that the BAF complex controls occupancy of AP-1¹⁵. In contrast, loss of EP400, which is part of the Sick With Rat8ts (SWR) complex, causes a large, 56 nt expansion of nucleosomes around AP-1 binding sites (p=10⁻⁴) (FIG. 16D).

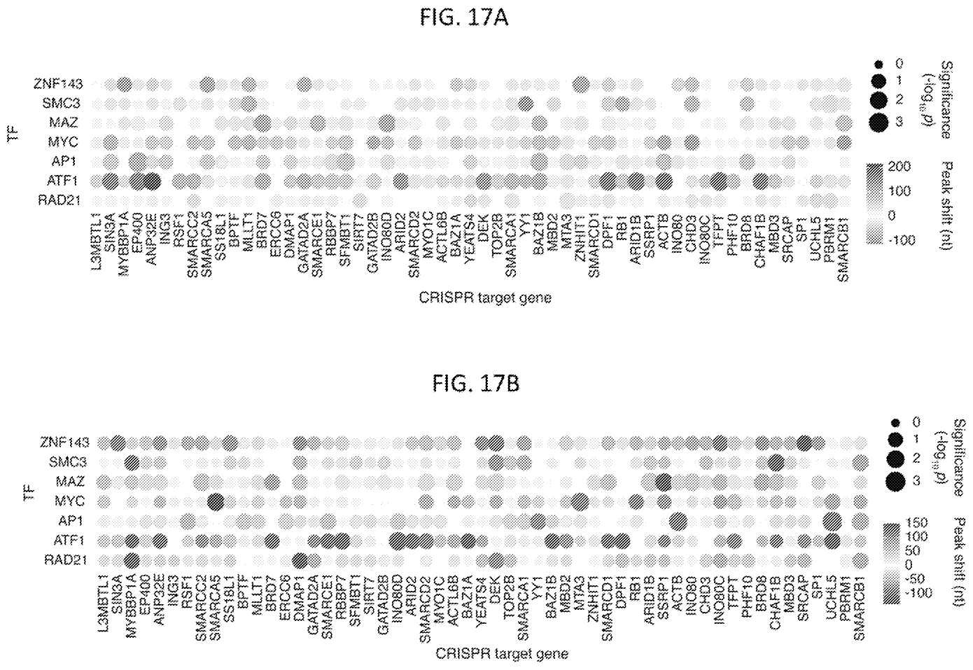

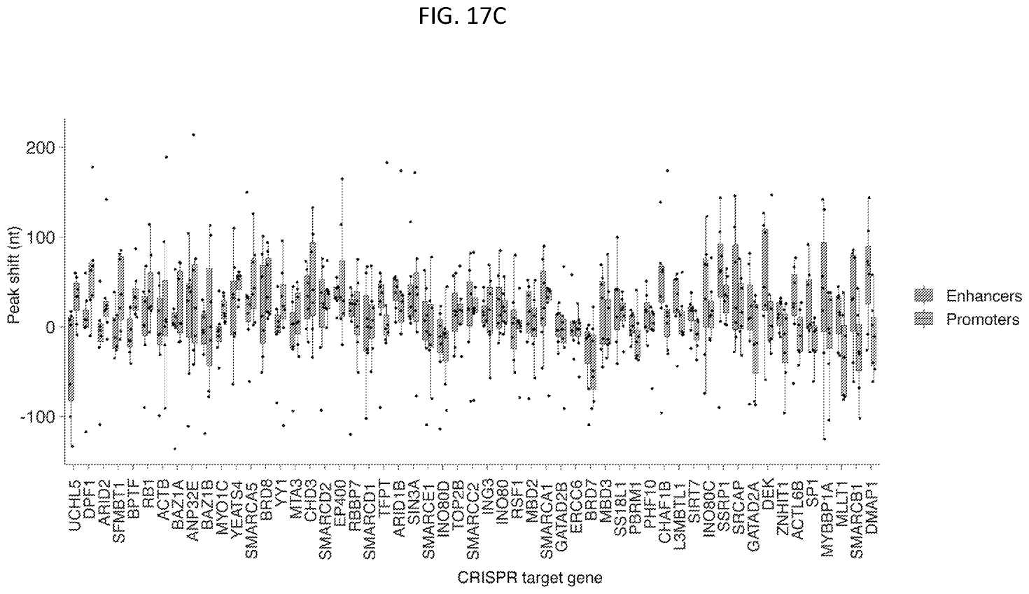

We further asked if there are specific differences in nucleosome dynamics surrounding TFBSs residing in enhancers versus promoters. We found that changes in nucleosome peak positions occur typically in either enhancers or promoters, depending on the specific TFBS. For example, across all CRISPR perturbations, the expansion of nucleosome spacing around AP-1 binding sites (FIG. 16B) occurs mostly in sites that are located in promoters (FIG. 16E). In contrast, expansion of nucleosome distances around ZNF143 binding sites occurs mostly in sites that are located in enhancers. An exception to this trend is found at ATF1 TFBS: Knock-out of chromatin remodelers results in nucleosome expansion around ATF1 binding sites in promoters, but compaction in ATF1 binding sites in enhancers (FIG. 16E, FIG. 17B and FIG. 17B).

Many gene knock-outs tend to cause more expansion in either enhancers or promoters (FIG. 17A-FIG. 17C). Knock-out of CoREST subunit SFMBT1 tends to cause nucleosome expansion around TFBSs in promoters but not in enhancers: for example, a 85 nt expansion around AP-1 binding sites in promoters and no change in nucleosomal positions around AP-1 binding sites in enhancers (FIG. 16F). In contrast, knock-out of BAF/pBAF subunit SMARCB1 tends to cause nucleosome expansion around TFBSs in enhancers but not in promoters: for example, a 82 nt expansion around RAD21 binding sites in enhancers but no change in nucleosomal positions around RAD21 binding sites in enhancers (FIG. 16G).

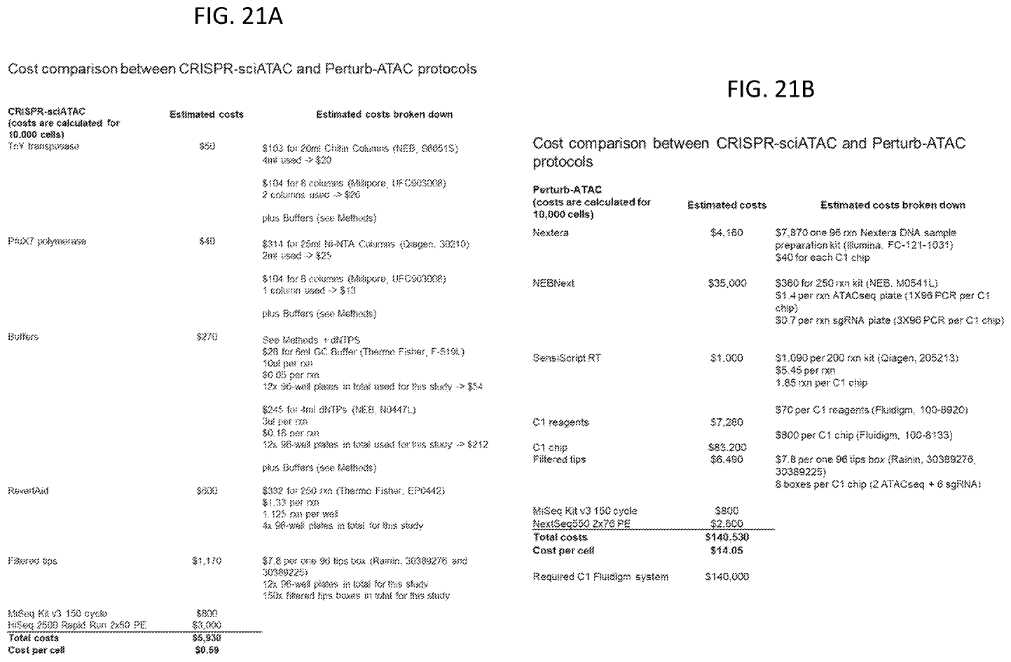

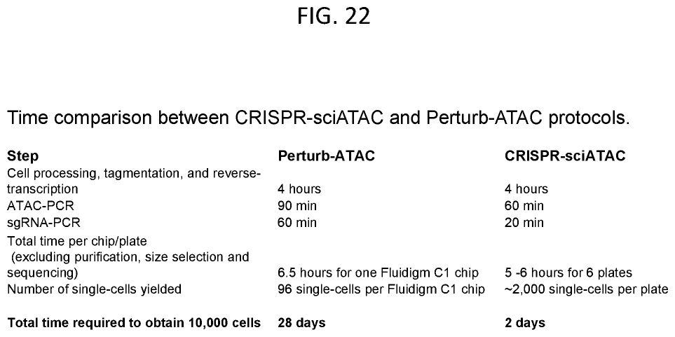

As demonstrated, CRISPRsciATAC allows for the joint capture of sgRNAs and ATAC profiles from single cells. We perturbed 105 genes using a library of 318 sgRNAs and investigated differential accessibility in histone marks and TFBSs following knock-out of chromatin modifiers. Using this method, we also showed that chromatin remodeling complexes could be perturbed in a uniform setting, thus avoiding batch effects. Implementing such a high throughput approach allows for the generation of data for less well-studied complexes, such as L3MBTL1 or CoREST, along with more well-studied complexes, such as SWI/SNF or INO80. Using the ATAC-seq profiles generated from our screen, we demonstrated that chromatin accessibility could be evaluated with high genomic resolution to show movement of nucleosomes in regulatory regions. Together, these results demonstrate that CRISPR-sciATAC can be used to correlate genotypes and chromatin architecture in a high-throughput manner. CRISPR-sciATAC offers an approach that takes advantage of two-step combinatorial indexing to label DNA molecules with unique cell barcodes and requires no specialized equipment. When compared with Perturb-ATAC, CRISPR-sciATAC can generate thousands of single cells at ˜20× less reagent cost and ˜14× less time required (FIG. 21A, FIG. 21B, and FIG. 22). It is also possible to combine CRISPR-sciATAC with droplet-based methods for even higher throughput and coverage. Overall, CRISPR-sciATAC can be applied to study diverse phenotypes and diseases and to understand interactions between genetic changes and genome-wide chromatin accessibility.

### REFERENCES

1. Guo, X., Chitale, P. & Sanjana, N. E. Target discovery for precision medicine using high-throughput genome engineering. in Advances in Experimental Medicine and Biology (2017).
2. Datlinger, P. et al. Pooled CRISPR screening with single-cell transcriptome readout. Nat. Methods (2017).
3. Adamson, B. et al. A Multiplexed Single-Cell CRISPR Screening Platform Enables Systematic Dissection of the Unfolded Protein Response. Cell (2016).
4. Dixit, A. et al. Perturb-Seq: Dissecting Molecular Circuits with Scalable Single-Cell RNA Profiling of Pooled Genetic Screens. Cell (2016).
5. Jaitin, D. A. et al. Dissecting Immune Circuits by Linking CRISPR-Pooled Screens with Single-Cell RNASeq. Cell (2016).
6. Flavahan, W. A., Gaskell, E. & Bernstein, B. E. Epigenetic plasticity and the hallmarks of cancer. Science (2017).
7. Cusanovich, D. A. et al. Multiplex single-cell profiling of chromatin accessibility by combinatorial cellular indexing. Science (2015).
8. Forbes, S. A. et al. COSMIC: Somatic cancer genetics at high-resolution. Nucleic Acids Res. (2017).
9. Kolde, R., Laur, S., Adler, P. & Vilo, J. Robust rank aggregation for gene list integration and meta-analysis. Bioinformatics (2012).
10. Wang, T. et al. Identification and characterization of essential genes in the human genome. Science (2015).
11. Klemm, S. L., Shipony, Z. & Greenleaf, W. J. Chromatin accessibility and the regulatory epigenome. Nat. Rev. Genet. (2019).
12. Mathelier, A. et al. JASPAR 2016: A major expansion and update of the open-access database of transcription factor binding profiles. Nucleic Acids Res. (2016).
13. Schep, A. N., Wu, B., Buenrostro, J. D. & Greenleaf, W. J. ChromVAR: Inferring transcription-factorassociated accessibility from single-cell epigenomic data. Nat. Methods (2017).
14. Kim, K. H. & Roberts, C. W. M. Targeting EZH2 in cancer. Nature Medicine (2016). doi:10.1038/nm.4036
15. Kelso, T. W. R. et al. Chromatin accessibility underlies synthetic lethality of SWI/SNF subunits in ARID1A-mutant cancers. Elife (2017).
16. Vierbuchen, T. et al. AP-1 Transcription Factors and the BAF Complex Mediate Signal-Dependent Enhancer Selection. Mol. Cell (2017).
17. Mathur, R. et al. ARID1A loss impairs enhancer-mediated gene regulation and drives colon cancer in mice. Nat. Genet. (2017).
18. Long, H. K., Prescott, S. L. & Wysocka, J. Ever-Changing Landscapes: Transcriptional Enhancers in Development and Evolution. Cell (2016).
19. Nord, A. S. et al. Rapid and pervasive changes in genome-wide enhancer usage during mammalian development. Cell (2013).
20. Ler, L. D. et al. Loss of tumor suppressor KDM6A amplifies PRC2-regulated transcriptional repression in bladder cancer and can be targeted through inhibition of EZH2. Sci. Transl. Med. (2017).
21. Margueron, R. & Reinberg, D. The Polycomb complex PRC2 and its mark in life. Nature (2011).
22. Xu, F. et al. Genomic loss of EZH2 leads to epigenetic modifications and overexpression of the HOX gene clusters in myelodysplastic syndrome. Oncotarget (2016).
23. Han, L. et al. Chromatin remodeling mediated by ARID1A is indispensable for normal hematopoiesis in mice. Leukemia (2019).
24. Thieme, S. et al. The histone demethylase UTX regulates stem cell migration and hematopoiesis. Blood (2013).
25. Koeffler, H. P. & Golde, D. W. Human myeloid leukemia cell lines: a review. Blood (1980).
26. Rubin, A. J. et al. Coupled Single-Cell CRISPR Screening and Epigenomic Profiling Reveals Causal Gene Regulatory Networks. Cell (2019).
27. Shalem, O. et al. Genome-scale CRISPR-Cas9 knockout screening in human cells. Science (2014).
28. Meier, J. A., Zhang, F. & Sanjana, N. E. GUIDES: SgRNA design for loss-of-function screens. Nature Methods (2017).
29. Picelli, S. et al. Tn5 transposase and tagmentation procedures for massively scaled sequencing projects. Genome Res. (2014).
30. Thompson, J. D., Higgins, D. G. & Gibson, T. J. CLUSTAL W: Improving the sensitivity of progressive multiple sequence alignment through sequence weighting, position-specific gap penalties and weight matrix choice. Nucleic Acids Res. (1994).
31. Goryshin, I. Y. & Reznikoff, W. S. Tn 5 in Vitro Transposition. J. Biol. Chem. (1998).
32. Norholm, M. H. H. A mutant Pfu DNA polymerase designed for advanced uracil-excision DNA engineering. BMC Biotechnol. (2010).
33. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods (2013).
34. Richter, K. N. et al. Glyoxal as an alternative fixative to formaldehyde in immunostaining and superresolution microscopy. EMBO J. (2017).
35. Adey, A. et al. In vitro, long-range sequence information for de novo genome assembly via transposase contiguity. Genome Res. (2014).
36. Amini, S. et al. Haplotype-resolved whole-genome sequencing by contiguity-preserving transposition and combinatorial indexing. Nat. Genet. (2014).
37. Langmead, B., Trapnell, C., Pop, M. & Salzberg, S. L. Ultrafast and memory-efficient alignment of short DNA sequences to the human genome. Genome Biol. (2009).
38. Langmead, B. & Salzberg, S. L. Fast gapped-read alignment with Bowtie 2. Nat Methods (2012).
39. Preissl, S. et al. Single-nucleus analysis of accessible chromatin in developing mouse forebrain reveals celltype-specific transcriptional regulation. Nature Neuroscience (2018).
40. Zhang, Y. et al. Model-based analysis of ChIP-Seq (MACS). Genome Biol. (2008).
41. Li, W. et al. MAGeCK enables robust identification of essential genes from genome-scale CRISPR/Cas9 knockout screens. Genome Biol. (2014).
42. Quinlan, A. R. & Hall, I. M. BEDTools: A flexible suite of utilities for comparing genomic features. Bioinformatics (2010).
43. Eden, E., Navon, R., Steinfeld, I., Lipson, D. & Yakhini, Z. GOrilla: a tool for discovery and visualization of enriched GO terms in ranked gene lists. BMC Bioinformatics (2009).
44. Karczewski, K. J. et al. Variation across 141,456 human exomes and genomes reveals the spectrum of lossof-function intolerance across human protein-coding genes. bioRxiv (2019).
45. Lek, M. et al. Analysis of protein-coding genetic variation in 60,706 humans. Nature (2016).
46. Vosa, U. et al. Unraveling the polygenic architecture of complex traits using blood eQTL meta-analysis. bioRxiv (2018).
47. Wei, Z., Zhang, W., Fang, H., Li, Y. & Wang, X. esATAC: an easy-to-use systematic pipeline for ATAC-seq data analysis. Bioinformatics (2018).

### SEQUENCE LISTING FREE TEXT

The following information is provided for sequences containing free text under numeric identifier <223>.

| SEQ ID NO: | |
|---|---|
| (containing | |
| free text) | Free text under <223> |
| 1 | <223> primer sequence |
| | <220> |
| | <221> misc_feature |
| | <222> (30) . . . (37) |
| | <223> n is a, c, g, or t |
| 2 | <223> primer sequence |
| 3 | <223> primer sequence |
| | <220> |
| | <221> misc_feature |
| | <222> (25) . . . (32) |
| | <223> n is a, c, g, or t |
| 4 | <223> primer sequence |
| | <220> |
| | <221> misc_feature |
| | <222> (28) . . . (35) |
| | <223> n is a, c, g, or t |
| 5 | <223> primer sequence |
| | <220> |
| | <221> misc_feature |
| | <222> (59) . . . (66) |
| | <223> n is a, c, g, or t |
| 6 | <223> primer sequence |
| 7 | <223> primer sequence |
| 8 | <223> primer sequence |
| 9 | <223> primer sequence |
| 10 | <223> primer sequence |
| | <220> |
| | <221> misc_feature |
| | <222> (15) . . . (22) |
| | <223> n is a, c, g, or t |
| 11 | <223> primer sequence |
| | <220> |
| | <221> misc feature |
| | <222> (16) . . . (23) |
| | <223> n is a, c, g, or t |
| 12 | <223> primer sequence |
| | <220> |
| | <221> misc_feature |
| | <222> (25) . . . (32) |
| | <223> n is a, c, g, or t |
| 13 | <223> primer sequence |
| 15 | <223> sarSeaEAK transposase |
| 18 | <223> Tn5 HA transposase |
| 24 | <223> Tn5 HA IE transposon sequence |
| 25 | <223> Tn5 HA OE transposon sequence |
| 26 | <223> Tn5 Nextera mosaic end sequence |
| 27 | <223> pMENT oligo sequence |
| | <220> |
| | <221> misc_feature |
| | <222> (1) . . . (1) |
| | <223> 5′ phosphate group |
| 28 | <223> spike-in T5 oligo sequence |
| 29 | <223> spike-in T7 oligo sequence |
| 30 | <223> spike-in P5 oligo sequence |
| 31 | <223> spike-in P7 oligo sequence |
| 32 | <223> T5 tagment sciATAC oligo sequence |
| 33 | <223> P5 sgR A PCR2 |
| 34 | <223> P5 sgR A PCR2 + 1 Stagger oligo sequence |
| 35 | <223> P5 sgR A PCR2 + 2 Stagger oligo sequence |
| 36 | <223> P5 sgR A PCR2 + 3 Stagger oligo sequence |
| 37 | <223> P5 sgR A PCR2 + Stagger oligo sequence |
| 38 | <223> U6 outer sgR A PCR1 oligo sequence |
| 39 | <223> Read 1 ATACseq oligo sequence |
| 40 | <223> Index 1 ATACseq oligo sequence |
| 41 | <223> Read 2 ATACseq oligo sequence |
| 42 | <223> ARID1A 1 sgRNA sequence |
| 43 | <223> ARID1A 2 sgRNA sequence |
| 44 | <223> ARID1A 3 sgRNA sequence |
| 45 | <223> ATRX 1 sgRNA sequence |
| 46 | <223> ATRX 2 sgRNA sequence |
| 47 | <223> ATRX 3 sgRNA sequence |
| 48 | <223> CHD4 1 sgRNA sequence |
| 49 | <223> CHD4 2 sgRNA sequence |
| 50 | <223> CHD4 3 sgRNA sequence |
| 51 | <223> CHD5 1 sgRNA sequence |
| 52 | <223> CHD5 2 sgRNA sequence |
| 53 | <223> CHD5 3 sgRNA sequence |
| 54 | <223> CHD8 1 sgRNA sequence |
| 55 | <223> CHD8 2 sgRNA sequence |
| 56 | <223> CHD8 3 sgRNA sequence |
| 57 | <223> DNMT3A 1 sgRNA sequence |
| 58 | <223> DNMT3A 2 sgRNA sequence |
| 59 | <223> DNMT3A 3 sgRNA sequence |
| 60 | <223> EZH2 1 sgRNA sequence |
| 61 | <223> EZH2 2 sgRNA sequence |
| 62 | <223> EZH2 3 sgRNA sequence |
| 63 | <223> H3F3A 1 sgRNA sequence |
| 64 | <223> H3F3A 2 sgRNA sequence |
| 65 | <223> H3F3A 3 sgRNA sequence |
| 66 | <223> H3F3B 1 sgRNA sequence |
| 67 | <223> H3F3B 2 sgRNA sequence |
| 68 | <223> H3F3B 3 sgRNA sequence |
| 69 | <223> HDAC9 1 sgRNA sequence |
| 70 | <223> HDAC9 2 sgRNA sequence |
| 71 | <223> HDAC9 3 sgRNA sequence |
| 72 | <223> HIST1H3B 1 sgRNA sequence |
| 73 | <223> HIST1H3B 2 sgRNA sequence |
| 74 | <223> HIST1H3B 3 sgRNA sequence |
| 75 | <223> ING1 1 sgRNA sequence |
| 76 | <223> ING1 2 sgRNA sequence |
| 77 | <223> ING1 3 sgRNA sequence |
| 78 | <223> KDM6A 1 sgRNA sequence |
| 79 | <223> KDM6A 2 sgRNA sequence |
| 80 | <223> KDM6A 3 sgRNA sequence |
| 81 | <223> MBD1 1 sgRNA sequence |
| 82 | <223> MBD1 2 sgRNA sequence |
| 83 | <223> MBD1 3 sgRNA sequence |
| 84 | <223> Non-targeting 1 sgRNA sequence |
| 85 | <223> Non-targeting 2 sgRNA sequence |
| 86 | <223> Non-targeting 3 sgRNA sequence |
| 87 | <223> PBRM1 1 sgRNA sequence |
| 88 | <223> PBRM1 2 sgRNA sequence |
| 89 | <223> PBRM1 3 sgRNA sequence |
| 90 | <223> PHF6 1 sgRNA sequence |
| 91 | <223> PHF6 2 sgRNA sequence |
| 92 | <223> PHF6 2 sgRNA sequence |
| 93 | <223> PRDM9 1 sgRNA sequence |
| 94 | <223> PRDM9 2 sgRNA sequence |
| 95 | <223> PRDM9 3 sgRNA sequence |
| 96 | <223> SETD2 1 sgRNA sequence |
| 97 | <223> SETD2 2 sgRNA sequence |
| 98 | <223> SETD2 3 sgRNA sequence |
| 99 | <223> SMARCA4 1 sgRNA sequence |
| 100 | <223> SMARCA4 2 sgRNA sequence |
| 101 | <223> SMARCA4 3 sgRNA sequence |
| 102 | <223> SMARCB1 1 sgRNA sequence |
| 103 | <223> SMARCB1 2 sgRNA sequence |
| 104 | <223> SMARCB1 3 sgRNA sequence |
| 105 | <223> TET2 1 sgRNA sequence |
| 106 | <223> TET2 2 sgRNA sequence |
| 107 | <223> TET2 3 sgRNA sequence |
| 108 | <223> TnY CDS (ViPAR P50K, M53Q) |

All documents cited in this specification, including patents, patent applications, publications, and websites, are incorporated herein by reference, as are the sequences and the text of the Sequence Listing (labeled “NYG-LIPP101PCT_ST25.txt”) filed herewith. U.S. Provisional Patent Application No. 62/873,494, filed Jul. 12, 2019, is also incorporated herein by reference in its entirety. While the invention has been described with reference to particular embodiments, it will be appreciated that modifications can be made without departing from the spirit of the invention. Such modifications are intended to fall within the scope of the appended claims.

1. An in vitro method for analyzing chromatin accessibility and RNA of each single cell in a library of cells, comprising:
(a) incubating cell nuclei in a suspension obtained from lysed cells with a tagmentation buffer that comprises a transposome complex,
wherein each cell nucleus comprises DNAs and RNAs from one cell,
wherein the transposome complex comprises a transposase, a transposon, and a first barcode,
wherein the transposase causes staggered double-stranded breaks in the DNAs, and
wherein the first barcode is ligated to the double-stranded DNA at the staggered break;
(b) performing reverse transcription which comprises contacting and incubating the cell nuclei of (a) with reverse transcription primers barcoded with the first barcode or the corresponding antisense sequence thereof, reverse transcriptase, and dNTPs in a reverse transcription buffer, whereby each of the RNAs is reverse transcribed to a DNA;
(c) sequencing DNA, which is extracted from digested cell nuclei of (b); and
(d) analyzing chromatin accessibility and RNA of the cells.

2. The method according to claim 1, wherein the first barcode is unique for each cell, whereby said DNA sequences acquired and analyzed with the same first barcode are identified as being from the same cell.

3. The method according to claim 1, further comprising:
(e) performing a combinatorial cellular indexing, which comprises
(i) transferring the cell nuclei to a first set of compartments prior to the tagmentation step of (a), wherein a total of n_{c} first-set compartments contain about nₙ nuclei per compartment;
(ii) transferring the cell nuclei to a second set of compartments after the step of (b) and prior to the step of (c), wherein a total of m_{c} second-set compartments contain about mₙ nuclei per compartment; and
(iii) barcoding each of the DNAs with a second barcode,
wherein the first barcode is unique for each first-set compartment, wherein the second barcode is unique for each second-set compartment, and wherein cell nuclei from the same first-set compartment are transferred to different second-set compartments, whereby sequences acquired and analyzed with the same combination of the first and the second barcodes are identified as being from the same cell.

4. The method according to claim 3, further comprising pooling the cell nuclei before the step of (e)(ii) and randomly distributing the pooled cell nuclei into the second set of compartments, wherein nₙ>>mₙ, optionally wherein n_{c}=96, nₙ=˜2000, m_{c}=96 to 1152, mₙ=15 to 20.

5. The method according to claim 1, wherein the first barcode comprises a third barcode to be ligated to the 5′ terminal of the DNA/RNA and a fourth barcode to be ligated to the 3′ terminal of the DNA/RNA.

6. The method according to claim 1, wherein the second barcode comprises a fifth barcode at the 5′ terminal of the DNA and a sixth barcode at the 3′ terminal of the DNA.

7. The method according to claim 1, wherein the cells are perturbed by a gain-of-function genomic editing, a loss-of-function genomic editing, a upregulation or downregulation of certain coding or non-coding genomic sequence, epigenome editing, RNAi, CRISPR-Cas, a chemical/biological agent, or a physical disturbance, prior to the cells being lysed and nuclei suspended.

8. The method according to claim 1, further comprising:
(f) a perturbation step comprising transducing the cells with one or more vectors, each vector comprising a nucleic acid sequence encoding a Cas protein in operative association with a first promoter which controls expression of the Cas protein, and a CRISPR guide RNA coding sequence in operative association with a second promoter which controls transcription thereof, and culturing the cells, wherein the RNA in the reverse transcription step (b) comprises the guide RNAs.

9. The method according to claim 8, wherein more than one CRISPR guide RNA transcribed from the vectors is targeted to each functional unit of a cell genome of interest.

10. The method according to claim 9, wherein each vector transcribes a single guide RNA and optionally there are at least 3 different guide RNAs targeted to each functional unit of a cell genome of interest.

11. The method according to claim 1, wherein the transposase is a TnY or Tn5.

12. The method according to claim 1, further comprising lysing the cells in a resuspension buffer comprising 0.1% Tween-20 and 0.1% Igepal CA630 prior to the incubation step (a).

13. The method according to claim 1, further comprising fixing the cells before lysis and optionally washing the fixed cells, wherein the cells are fixed via suspended in a fixation buffer, and wherein the fixation buffer comprises about 20% (v/v) ethanol and about 3.1% (v/v) glyoxal at a pH of about 5.0, optionally, the fixation buffer is made by mixing 280 parts of H₂O, 79 parts of 100% ethanol, 31 parts of 40% glyoxal, and 3 parts of glacial acetic acid, and adjusting pH to about 5.0 and the final volume to about 400 parts using NaOH.

14. The method according to claim 13, wherein the cells are fixed for 7 minutes at room temperature.

15. The method according to claim 1, wherein the tagmentation buffer comprises H₂O, 5 mM Mg²⁺, a hydrophilic solvent in a zwitterionic buffer at a pH of about 8.5.

16. The method according to claim 1, wherein the tagmentation buffer is 50 mM TAPS-NaOH at pH 8.5, 25 mM MgCl₂, 50% DMF and RNase Inhibitor.

17. (canceled)

18. The method according to claim 1, wherein the transposome complex and the cell nuclei are incubated for 30 minutes at 37° C. in step (a).

19. The method according to claim 1, wherein the tagmentation step of (a) further comprises one or both
(i) adding EDTA, whereby the tagmentation reaction is stopped, and
(ii) quenching the EDTA by adding MgCl₂.

20. (canceled)

21. The method according to claim 1, further comprising performing RNA-seq, a mitochondrial RNA assay, or ATAC-seq.

22. An in vitro method for analyzing chromatin accessibility and RNA of each single cell in a library of cells, comprising:
(a) a preparation step which comprises
(i) lysing the cells to release nuclei therefrom; and
(ii) suspending the cell nuclei of (a)(i) in a tagmentation buffer, wherein each cell nucleus comprises DNAs and RNAs from one cell;
(b) a tagmentation step which comprises
(i) incubating a transposome complex with the cell nuclei in the tagmentation buffer of (a)(ii), wherein the transposome complex comprises a transposase, a transposon and a first barcode, wherein the transposase causes staggered double-stranded breaks in the DNAs, and wherein the first barcode is ligated to the double-stranded DNA at the staggered break;
(c) a reverse transcription step which comprises
(i) contacting and incubating the cell nuclei of (b) with reverse transcription primers barcoded with the first barcode or the corresponding antisense sequence thereof, reverse transcriptase and dNTPs in a reverse transcription buffer, whereby each of the RNAs is reverse transcribed to a DNA; and
(d) a sequencing step which comprises
(i) digesting the cell nuclei and extracting DNAs; and
(ii) sequencing the DNAs extracted and analyzing chromatin accessibility and RNA of the cells.


SEQUENCE LISTING (108 sequences)

SEQ ID NO: 1 - DNA, 51 bp, Artificial Sequence
  primer sequence
  misc_feature (30)..(37): n is a, c, g, or t
aatgatacggcgaccaccgagatctacacnnnnnnnntcgtcggcagcgtc

SEQ ID NO: 2 - DNA, 63 bp, Artificial Sequence
  primer sequence
tcgtcggcagcgtctccacgcaaccaacgcaccgtctccgcctcagatgtgtataagagacag

SEQ ID NO: 3 - DNA, 47 bp, Artificial Sequence
  primer sequence
  misc_feature (25)..(32): n is a, c, g, or t
caagcagaagacggcatacgagatnnnnnnnngtctcgtgggctcgg

SEQ ID NO: 4 - DNA, 69 bp, Artificial Sequence
  primer sequence
  misc_feature (28)..(35): n is a, c, g, or t
gtctcgtgggctcggctgtccctgtccnnnnnnnngcgatcgaggacggcagatgtgtataagagacag

SEQ ID NO: 5 - DNA, 80 bp, Artificial Sequence
  primer sequence
  misc_feature (59)..(66): n is a, c, g, or t
aatgatacggcgaccaccgagatctacactctttccctacacgacgctcttccgatctnnnnnnnncgccgtccagctga

SEQ ID NO: 6 - DNA, 75 bp, Artificial Sequence
  primer sequence
aatgatacggcgaccaccgagatctacactctttccctacacgacgctcttccgatctgtacgccgtccagctga

SEQ ID NO: 7 - DNA, 74 bp, Artificial Sequence
  primer sequence
aatgatacggcgaccaccgagatctacactctttccctacacgacgctcttccgatcttacgccgtccagctga

SEQ ID NO: 8 - DNA, 73 bp, Artificial Sequence
  primer sequence
aatgatacggcgaccaccgagatctacactctttccctacacgacgctcttccgatcttcgccgtccagctga

SEQ ID NO: 9 - DNA, 72 bp, Artificial Sequence
  primer sequence
aatgatacggcgaccaccgagatctacactctttccctacacgacgctcttccgatctcgccgtccagctga

SEQ ID NO: 10 - DNA, 37 bp, Artificial Sequence
  primer sequence
  misc_feature (15)..(22): n is a, c, g, or t
cgccgtccagctgannnnnnnnccgggagctgcatgt

SEQ ID NO: 11 - DNA, 39 bp, Artificial Sequence
  primer sequence
  misc_feature (16)..(23): n is a, c, g, or t
ccgggagctgcatgtnnnnnnnntttttcaagttgataa

SEQ ID NO: 12 - DNA, 52 bp, Artificial Sequence
  primer sequence
  misc_feature (25)..(32): n is a, c, g, or t
caagcagaagacggcatacgagatnnnnnnnnttgtggaaaggacgaaacac

SEQ ID NO: 13 - DNA, 26 bp, Artificial Sequence
  primer sequence
tgcatatacgatcacaaggctgttag

SEQ ID NO: 14 - protein, 66 aa, Photorhabdus luminescens
MFSTSAEQWANDTFQHAELGDKRRTNRLVKVACSLANHIGQSLVQSLDSPADVEAAYRLTRNSAIA

SEQ ID NO: 15 - protein, 63 aa, Artificial Sequence
  sarSeaEAK transposase
MDPEQWAQCQFGHANLNDPRRTQRLVSLATSITQQPGVAVSKLPLSPAEMEGAYRFIRNENIQ

SEQ ID NO: 16 - protein, 66 aa, Vibrio campbellii
MTHSDAKLWAQEQFGQAQLKDPRRTQRLISLATSIANQPGVSVAKLPFSPADMEGAYRFIRNENIN

SEQ ID NO: 17 - protein, 66 aa, Vibrio parahaemolyticus
MTHSDAKLWAQEQFGQAQLKDPRRTQRLISLATSIANQPGVSVAKLPFSPADMEGAYRFIRNDNID

SEQ ID NO: 18 - protein, 70 aa, Artificial Sequence
  Tn5 HA transposase
MITSALHRAADWAKSVFSSAALGDPRRTARLVNVAAQLAKYSGKSITISSEGSKAMQEGAYRFIRNPNVS

SEQ ID NO: 19 - protein, 67 aa, Candidatus glomeribacter
MFRREAGDWAHQTFGECNLGDERRTKRLVEVGKRLANQIGCSLPKCCEGDKAALLGSYRLLRNDAVN

SEQ ID NO: 20 - protein, 69 aa, Legionella longbeachae
MDLAIEDAAAWSEAIFGSVDLGDKRLTRRLTQIGKQLSSMPGGSLPESCEGQDALIEGSYRFLRNKRVT

SEQ ID NO: 21 - protein, 69 aa, Legionella pneumophila
MDLAIEDAAAWSEAIFGSVALGDKRLTRRLIQIGKQLSSIPGGSLSESCEGQDALIEGSYRFLRNKRVT

SEQ ID NO: 22 - DNA, 19 bp, Vibrio parahaemolyticus
acttgtgatcaagagacag

SEQ ID NO: 23 - DNA, 19 bp, Vibrio parahaemolyticus
atttgtgtataagagacag

SEQ ID NO: 24 - DNA, 19 bp, Artificial Sequence
  Tn5 HA IE transposon sequence
agatctgatcaagagacag

SEQ ID NO: 25 - DNA, 19 bp, Artificial Sequence
  Tn5 HA OE transposon sequence
acttgtgtataagagtcag

SEQ ID NO: 26 - DNA, 19 bp, Artificial Sequence
  Tn5 Nextera mosaic end sequence
agatgtgtataagagacag

SEQ ID NO: 27 - DNA, 19 bp, Artificial Sequence
  pMENT oligo sequence
  misc_feature (1)..(1): 5′ phosphate group
ctgtctcttatacacatct

SEQ ID NO: 28 - DNA, 63 bp, Artificial Sequence
  spike-in T5 oligo sequence
gacgctgccgacgaaggtgcgtatagcctcaccgtctccgcctcagatgtgtataagagacag

SEQ ID NO: 29 - DNA, 69 bp, Artificial Sequence
  spike-in T7 oligo sequence
cagagcacccgagccgacagggacaggcgagtaatgcgatcgaggacggcagatgtgtataagagacag

SEQ ID NO: 30 - DNA, 51 bp, Artificial Sequence
  spike-in P5 oligo sequence
aatgatacggcgaccaccgagatctacactcctacgggacgctgccgacga

SEQ ID NO: 31 - DNA, 47 bp, Artificial Sequence
  spike-in P7 oligo sequence
caagcagaagacggcatacgagatctaagaaccagagcacccgagcc

SEQ ID NO: 32 - DNA, 63 bp, Artificial Sequence; identical to SEQ ID NO: 2
  T5 tagment sciATAC oligo sequence

SEQ ID NO: 33 - DNA, 72 bp, Artificial Sequence; identical to SEQ ID NO: 9
  P5 sgR A PCR2

SEQ ID NO: 34 - DNA, 73 bp, Artificial Sequence
  P5 sgR  A PCR2 +1 Stagger oligo sequence
aatgatacggcgaccaccgagatctacactctttccctacacgacgctcttccgatctacgccgtccagctga

SEQ ID NO: 35 - DNA, 74 bp, Artificial Sequence; identical to SEQ ID NO: 7
  P5 sgR  A PCR2 +2 Stagger oligo sequnce

SEQ ID NO: 36 - DNA, 75 bp, Artificial Sequence; identical to SEQ ID NO: 6
  P5 sgR  A PCR2 +3 Stagger oligo sequence

SEQ ID NO: 37 - DNA, 72 bp, Artificial Sequence; identical to SEQ ID NO: 9
  P5 sgR A PCR2 + Stagger oligo sequence

SEQ ID NO: 38 - DNA, 26 bp, Artificial Sequence
  U6 outer sgR A PCR1 oligo sequence
tgcatatacgatcacaaggctgttag

SEQ ID NO: 39 - DNA, 34 bp, Artificial Sequence
  Read 1 ATACseq oligo sequence
caccgtctccgcctcagatgtgtataagagacag

SEQ ID NO: 40 - DNA, 34 bp, Artificial Sequence
  Index 1 ATACseq oligo sequence
ctgtctcttatacacatctgccgtcctcgatcgc

SEQ ID NO: 41 - DNA, 34 bp, Artificial Sequence
  Read 2 ATACseq oligo sequence
gcgatcgaggacggcagatgtgtataagagacag

SEQ ID NO: 42 - DNA, 20 bp, Artificial Sequence
  ARID1A 1 sgRNA sequence
ccgttatctggccttcactg

SEQ ID NO: 43 - DNA, 20 bp, Artificial Sequence
  ARID1A 2 sgRNA sequence
gcgagactgagcaacactgg

SEQ ID NO: 44 - DNA, 20 bp, Artificial Sequence
  ARID1A 3 sgRNA sequence
cacatccctgacccaacctg

SEQ ID NO: 45 - DNA, 20 bp, Artificial Sequence
  ATRX 1 sgRNA sequence
cacatccctgacccaacctg

SEQ ID NO: 46 - DNA, 20 bp, Artificial Sequence
  ATRX 2 sgRNA sequence
tagtctagctgcaaacacca

SEQ ID NO: 47 - DNA, 20 bp, Artificial Sequence
  ATRX 3 sgRNA sequence
tcagtgtgcagattctacca

SEQ ID NO: 48 - DNA, 20 bp, Artificial Sequence
  CHD4 1 sgRNA sequence
tggcttgtaagagacctgcg

SEQ ID NO: 49 - DNA, 20 bp, Artificial Sequence
  CHD4 2 sgRNA sequence
cattgaccacgtgttctcag

SEQ ID NO: 50 - DNA, 20 bp, Artificial Sequence
  CHD4 3 sgRNA sequence
ttcaagggtgaaatgaaccg

SEQ ID NO: 51 - DNA, 20 bp, Artificial Sequence
  CHD5 1 sgRNA sequence
gtggacgacgacaatcccgg

SEQ ID NO: 52 - DNA, 20 bp, Artificial Sequence
  CHD5 2 sgRNA sequence
acacgcggcagaactccatg

SEQ ID NO: 53 - DNA, 20 bp, Artificial Sequence
  CHD5 3 sgRNA sequence
cgttctcccgaatcaccgag

SEQ ID NO: 54 - DNA, 20 bp, Artificial Sequence
  CHD8 1 sgRNA sequence
tacacagtaagaagacaaag

SEQ ID NO: 55 - DNA, 20 bp, Artificial Sequence
  CHD8 2 sgRNA sequence
tgagtctcacagtattgaca

SEQ ID NO: 56 - DNA, 20 bp, Artificial Sequence
  CHD8 3 sgRNA sequence
tgccctatgaggatagcaca

SEQ ID NO: 57 - DNA, 20 bp, Artificial Sequence
  DNMT3A 1 sgRNA sequence
aaagccccggaagagcacag

SEQ ID NO: 58 - DNA, 20 bp, Artificial Sequence
  DNMT3A 2 sgRNA sequence
tgacacgccaaaggaccctg

SEQ ID NO: 59 - DNA, 20 bp, Artificial Sequence
  DNMT3A 3 sgRNA sequence
gtaccgcaaagccatctacg

SEQ ID NO: 60 - DNA, 20 bp, Artificial Sequence
  EZH2 1 sgRNA sequence
cggctccacaagtaagacag

SEQ ID NO: 61 - DNA, 20 bp, Artificial Sequence
  EZH2 2 sgRNA sequence
caagggcacgaactgtcaca

SEQ ID NO: 62 - DNA, 20 bp, Artificial Sequence
  EZH2 3 sgRNA sequence
attgctggcaccatctgacg

SEQ ID NO: 63 - DNA, 20 bp, Artificial Sequence
  H3F3A 1 sgRNA sequence
cgaccggtggtaaagcaccc

SEQ ID NO: 64 - DNA, 20 bp, Artificial Sequence
  H3F3A 2 sgRNA sequence
aagcacgttctccacgtatg

SEQ ID NO: 65 - DNA, 20 bp, Artificial Sequence
  H3F3A 3 sgRNA sequence
aagcaccgatagctgcgctc

SEQ ID NO: 66 - DNA, 20 bp, Artificial Sequence
  H3F3B 1 sgRNA sequence
tatcttcgaacagacccacc

SEQ ID NO: 67 - DNA, 20 bp, Artificial Sequence
  H3F3B 2 sgRNA sequence
cttcttcaccccgccggtag

SEQ ID NO: 68 - DNA, 20 bp, Artificial Sequence
  H3F3B 3 sgRNA sequence
aggaaaagcgctccctctac

SEQ ID NO: 69 - DNA, 20 bp, Artificial Sequence
  HDAC9 1 sgRNA sequence
tgctgggtgaggtaaaacag

SEQ ID NO: 70 - DNA, 20 bp, Artificial Sequence
  HDAC9 2 sgRNA sequence
acggacaacagggtccacca

SEQ ID NO: 71 - DNA, 20 bp, Artificial Sequence
  HDAC9 3 sgRNA sequence
tggagaactgctactgactg

SEQ ID NO: 72 - DNA, 20 bp, Artificial Sequence
  HIST1H3B 1 sgRNA sequence
ctcgtactaaacagacagct

SEQ ID NO: 73 - DNA, 20 bp, Artificial Sequence
  HIST1H3B 2 sgRNA sequence
aaagcctcaccgttaccgcc

SEQ ID NO: 74 - DNA, 20 bp, Artificial Sequence
  HIST1H3B 3 sgRNA sequence
gaagatcggtcttgaagtct

SEQ ID NO: 75 - DNA, 20 bp, Artificial Sequence
  ING1 1 sgRNA sequence
atgtccttatcattcccctg

SEQ ID NO: 76 - DNA, 20 bp, Artificial Sequence
  ING1 2 sgRNA sequence
gaaccaaccacaccgcaggg

SEQ ID NO: 77 - DNA, 20 bp, Artificial Sequence
  ING1 3 sgRNA sequence
ggtactgtcccaagtgccgg

SEQ ID NO: 78 - DNA, 20 bp, Artificial Sequence
  KDM6A 1 sgRNA sequence
ggcaattaaagcatttcagg

SEQ ID NO: 79 - DNA, 20 bp, Artificial Sequence
  KDM6A 2 sgRNA sequence
ataggcctgtaaagcatcca

SEQ ID NO: 80 - DNA, 20 bp, Artificial Sequence
  KDM6A 3 sgRNA sequence
aggcactgttcattgggttc

SEQ ID NO: 81 - DNA, 20 bp, Artificial Sequence
  MBD1 1 sgRNA sequence
tgcgacaagcccaaattcgg

SEQ ID NO: 82 - DNA, 20 bp, Artificial Sequence
  MBD1 2 sgRNA sequence
aagagggtgagatcacacgc

SEQ ID NO: 83 - DNA, 20 bp, Artificial Sequence
  MBD1 3 sgRNA sequence
tgtcagacccaagaggattg

SEQ ID NO: 84 - DNA, 20 bp, Artificial Sequence
  Non-targeting 1 sgRNA sequence
atctcaagaccattgcccag

SEQ ID NO: 85 - DNA, 20 bp, Artificial Sequence
  Non-targeting 2 sgRNA sequence
atacattgaaggactcagtg

SEQ ID NO: 86 - DNA, 20 bp, Artificial Sequence
  Non-targeting 3 sgRNA sequence
gaagaatacagacaactcag

SEQ ID NO: 87 - DNA, 20 bp, Artificial Sequence
  PBRM1 1 sgRNA sequence
gtacttcaggagattaaacg

SEQ ID NO: 88 - DNA, 20 bp, Artificial Sequence
  PBRM1 2 sgRNA sequence
ctttatcatgcaatgcacag

SEQ ID NO: 89 - DNA, 20 bp, Artificial Sequence
  PBRM1 3 sgRNA sequence
aagacttaccattaccactg

SEQ ID NO: 90 - DNA, 20 bp, Artificial Sequence
  PHF6 1 sgRNA sequence
aaagggtcatgcatacaaag

SEQ ID NO: 91 - DNA, 20 bp, Artificial Sequence
  PHF6 2 sgRNA sequence
tccttggatggccttaagag

SEQ ID NO: 92 - DNA, 20 bp, Artificial Sequence
  PHF6 3 sgRNA sequence
gcaaatttactgaatgcaag

SEQ ID NO: 93 - DNA, 20 bp, Artificial Sequence
  PRDM9 1 sgRNA sequence
gatcagaaagagagcgacgg

SEQ ID NO: 94 - DNA, 20 bp, Artificial Sequence
  PRDM9 2 sgRNA sequence
agaacagccagataaaacag

SEQ ID NO: 95 - DNA, 20 bp, Artificial Sequence
  PRDM9 3 sgRNA sequence
cgagctgtcttccagttggg

SEQ ID NO: 96 - DNA, 20 bp, Artificial Sequence
  SETD2 1 sgRNA sequence
cttgtaagacaccttcacca

SEQ ID NO: 97 - DNA, 20 bp, Artificial Sequence
  SETD2 2 sgRNA sequence
cagtggacgtcagctcagcg

SEQ ID NO: 98 - DNA, 20 bp, Artificial Sequence
  SETD2 3 sgRNA sequence
cgtggatcaccttcaccggg

SEQ ID NO: 99 - DNA, 20 bp, Artificial Sequence
  SMARCA4 1 sgRNA sequence
gcagatcgagtcctacccca

SEQ ID NO: 100 - DNA, 20 bp, Artificial Sequence
  SMARCA4 2 sgRNA sequence
tcttcttgtctcggcccatg

SEQ ID NO: 101 - DNA, 20 bp, Artificial Sequence
  SMARCA4 3 sgRNA sequence
tgagaacgcatctcagcccg

SEQ ID NO: 102 - DNA, 20 bp, Artificial Sequence
  SMARCB1 1 sgRNA sequence
tgcggaattgatctgccctg

SEQ ID NO: 103 - DNA, 20 bp, Artificial Sequence
  SMARCB1 2 sgRNA sequence
gggctggatccagaaacctg

SEQ ID NO: 104 - DNA, 20 bp, Artificial Sequence
  SMARCB1 3 sgRNA sequence
agaatttggaggaaaacctg

SEQ ID NO: 105 - DNA, 20 bp, Artificial Sequence
  TET2 1 sgRNA sequence
ccgttatctggccttcactg

SEQ ID NO: 106 - DNA, 20 bp, Artificial Sequence
  TET2 2 sgRNA sequence
gcgagactgagcaacactgg

SEQ ID NO: 107 - DNA, 20 bp, Artificial Sequence
  TET2 3 sgRNA sequence
cacatccctgacccaacctg

SEQ ID NO: 108 - DNA, 1377 bp, Artificial Sequence
  TnY CDS (ViPAR P50K, M53Q)
atgacccactccgatgcgaaactgtgggctcaggagcaattcggtcaggcccaactgaaagatccgcgccgcacccagcgcctgatttctctggcgaccagcattgctaaccagccgggtgttagcgttgcgaaactgccgttttctaaagccgatcaggagggcgcgtaccgtttcattcgtaacgataacatcgacgcgaaagacatcgctgaagcaggctttcagtccaccgtatcccgcgctaacgaacacaaagagctgctggcgctggaagacactacgaccctgtctttcccgcatcgttccatcaaagaagaactgggccatacgaaccagggtgatcgcacccgcgccctgcacgttcactctaccctgctgttcgcgccgcagaaccagactatcgtgggtctgatcgagcagcagcgttggtctcgtgatattactaaacgcggtcagaaacatcagcacgctacccgtccttataaagaaaaagaatcctataaatgggagcaggcttcccgtcgtgttgtggagcgcctgggtgataaaatgctggatgtcatttctgtttgcgaccgcgaggcagatctgtttgaatacctgacctacaaacgtcaacaccagcagcgtttcgttgttcgtagcatgcagtctcgctgtctggaagaacacgctcagaaactgtatgactacgcacaggcgctgccatctgtaaaaacgaaggcactgaccatccctcaaaaaggtggccgtaaagcacgtgacgttaaactggacgttaaatacggccaggttactctgaaagcgccggccaacaaaaaggagcacgcaggcattccggtttactacgtgggctgcctggaacagggtacttccaaagataaactggcgtggcacctgctgacctctgaacctattaacaacgtcgaggatgccatgcgtatcatcggctactacgaacgtcgttggctgatcgaggattttcacaaagtatggaaatccgaaggtactgacgtagaatccctgcgtctgcagagcaaagacaacctggaacgtctgtccgttatctacgcgtttgttgctacccgcctgctggcactgcgttttatcaaggaagttgatgaactgaccaaagaaagctgtgaaaaagttctgggccagaaagcgtggaaactgctgtggctgaagctggaatctaaaaccctgccgaaagaggtaccggacatgggttgggcttataaaaacctggctaaactgggtggctggaaggacactaagcgtaccggtcgcgcttctatcaaagttctgtgggagggttggttcaaactgcagaccatcctggagggctatgaactggcgatgtccctggaccac